SEQUENCE LISTING (173 sequences)

SEQ ID NO: 1 - protein, 422 aa, Jeotgalicoccus sp.
MATLKRDKGLDNTLKVLKQGYLYTTNQRNRLNTSVFQTKALGGKPFVVVTGKEGAEMFYNNDVVQREGMLPKRIVNTLFGKGAIHTVDGKKHVDRKALFMSLMTEGNLNYVRELTRTLWHANTQRMESMDEVNIYRESIVLLTKVGTRWAGVQAPPEDIERIATDMDIMIDSFRALGGAFKGYKASKEARRRVEDWLEEQIIETRKGNIHPPEGTALYEFAHWEDYLGNPMDSRTCAIDLMNTFRPLIAINRFVSFGLHAMNENPITREKIKSEPDYAYKFAQEVRRYYPFVPFLPGKAKVDIDFQGVTIPAGVGLALDVYGTTHDESLWDDPNEFRPERFETWDGSPFDLIPQGGGDYWTNHRCAGEWITVIIMEETMKYFAEKITYDVPEQDLEVDLNSIPGYVKSGFVIKNVREVVDRT

SEQ ID NO: 2 - protein, 425 aa, Micrococcus lylae
MSKRVPKDRGIDNSLKIMKEGYEYVPARMKKFNTNIFETRVLGGKTAVVISGKDAAELFYDNDKTERKGTLPKRVVKTLFGKGAIHTTTGKKHIDRKALFMSLMTDENLAYLRKLTRIYWFQNIEHMQYKQKVNVYEEATELLTKVGLRWAGIVDHPENIQKMADDMNKMIDSFSAIGSLYGGYREAKKARARVEQFLEDQITAVRKGKIHPEKGTALYEFSHWEDMNGKPMDARLCAVDLMNVIRPLVAINKFVSFGVLALHEFPGERVRVALNEGDYAYKFVQEVRRYYPFVPFLPGKAKENITFDGYKIQKDTMMLLDIYGTLHRDDLFSEPERFNPYRFDNWDGSPFDLIPQGGGDYYTNHRCAGEWMTIIIMEETMKFFANEISYDVPPQDFTVDTTKFPGKVASGMDIENIRVNIDRTK

SEQ ID NO: 3 - protein, 425 aa, Unknown
  Artificial codon optimized gene sequence
MSKRVPKDRGIDNSLKIMKEGYEYVPARMKKFNTNIFETRVLGGKTAVVISGKDAAELFYDNDKTERKGTLPKRVVKTLFGKGAIHTTTGKKHIDRKALFMSLMTDENLAYLRKLTRIYWFQNIEHMQYKQKVNVYEEATELLTKVGLRWAGIVDHPENIQKMADDMNKMIDSFSAIGSLYGGYREAKKARARVEQFLEDQITAVRKGKIHPEKGTALYEFSHWEDMNGKPMDARLCAVDLMNVIRPLVAINKFVSFGVLALHEFPGERVRVALNEGDYAYKFVQEVRRYYPFVPFLPGKAKENITFDGYKIQKDTMMLLDIYGTLHRDDLFSEPERFNPYRFENWDGSPFDLIPQGGGDYYTNHRCAGEWMTIIIMEETMKFFANEISYDVPPQDFTVDTTKFPGKVASGMDIENIRVNIDRTK

SEQ ID NO: 4 - protein, 425 aa, Macrococcus caseolyticus
MSKRVPKDRGIDNSLKIMKEGYEYVPARMKKFNTNIFETRVLGGKTAVVISGKDAAELFYDNDKTERKGTLPKRVVKTLFGKGAIHTTTGKKHIDRKVLFMSLMTDENLAYLRKLTRIYWFQNIEHMQYKQKVNVYEEATELLTKVGLRWAGIVDHPENIQKMADDMNKMIDSFSAIGSLYGGYREAKKARARVEQFLEDQITAVRKGKIHPEKGTALYEFSHWEDMNGKPMDARLCAVDLMNVIRPLVAINKFVSFGVLALHEFPGERVRVALNEGDYAYKFVQEVRRYYPFVPFLPGKAKENITFDGYKIQKDTMMLLDIYGTLHRDDLFSEPERFNPYRFENWDGSPFDLIPQGGGDYYTNHRCAGEWMTIIIMEETMKFFANEISYDVPPQDFTVDTTKFPGKVASGMDIENIRVNIDRTK

SEQ ID NO: 5 - protein, 425 aa, Macrococcus caseolyticus
MSKRVPKDRGIDNSLKIMKEGYEYVPARMKKFNTNIFETRVLGGKTAVVISGKEAAELFYDNDKTERKGTLPKRVVKTLFGKGAIHTTTGKKHIDRKALFMSLMTDENLAYLRKLTRIYWFQNIEHMQYKQKVNVYEEATELLTKVGLRWAGIVDHPENIQKMADDMNKMIDSFSAIGSLYGGYREAKKARARVEQFLEDQITAVRKGKIHPEKGTALYEFSHWEDMNGKPMDARLCAVDLMNVIRPLVAINKFVSFGVLALHEFPGERVRVALNEGDYAYKFVQEVRRYYPFVPFLPGKAKENITFDGYKIQKDTMMLLDIYGTLHRDDLFSEPERFNPYRFENWDGSPFDLIPQGGGDYYTNHRCAGEWMTIIIMEETMKFFANEISYDVPPQDFTVDTTKFPGKVASGMDIENIRVNIDRTK

SEQ ID NO: 6 - protein, 425 aa, Macrococcus caseolyticus
MSKRVPKDRGIDNSLKIMKEGYEYVPARMKKFNTNIFETRVLGGKTAVVISGKDAAKLFYDNDKTERKGTLPKRVVKTLFGKGAIHTTTGKKHIDRKALFMSLMTDENLAYLRKLTRIYWFQNIEHMQYKQKVNVYEEATELLTKVGLRWAGIVDHPENIQKMADDMNKMIDSFSAIGSLYGGYREAKKARARVEQFLEDQITAVRKGKIHPEKGTALYEFSHWEEMNGKPMDARLCAVDLMNVIRPLVAINKFVSFGVLALHEFPGERVRVALNEGDYAYKFVQEVRRYYPFVPFLPGKAKENITFDGYKIQKDTMMLLDIYGTLHRDDLFSEPERFNPYRFENWDGSPFDLIPQGGGDYYTNHRCAGEWMTIIIMEETMKFFANEISYDVPPQDFTVDTTKFPGKVASGMDIENIRVNIDRTK

SEQ ID NO: 7 - protein, 425 aa, Macrococcus caseolyticus
MSKRIPKDRGIDNSLKIMKEGYEYVPARMKKFNTNIFETRVLGGKTAVVISGKDAAELFYDNDKTERKGTLPKRVVKTLFGKGAIHTTTGKKHIDRKVLFMSLMTDENLAYLRKLTRIYWFQNIEHMQYKQKVNVYEEATELLTKVGLRWAGIVDHPENIQKMADDMNKMIDSFSAIGSLYGGYREAKKARARVEQFLEDQITAVRKGKIHPEKGTALYEFSHWEDMNGKPMDARLCAVDLMNVIRPLVAINKFVSFGVLALHEFPGERVRVALNEGDYAYKFVQEVRRYYPFVPFLPGKAKENITFDGYKIQKDTMMLLDIYGTLHRDDLFSEPERFNPYRFENWDGSPFDLIPQGGGDYYTNHRCAGEWMTIIIMEETMKFFANEISYDVPPQDFTVDTTKFPGKVASGMDIENIRVNIDRTK

SEQ ID NO: 8 - protein, 425 aa, Macrococcus caseolyticus
MSKRVPKDRGIDNSLKIMKEGYEYVPARMKKFNTNIFETRVLGGKTAVVISGKDAAELFYDNDKTERKGTLPKRVVKTLFGKGAIHTTTGKKHIDRKALFMSLMTDENLAYLRKLTRIYWFQNIEHMQYKQKVNVYEEATELLTKVGLRWAGIVDHPENIQKMADDMNKMIDSFSAIGSLYGGYREAKKARARVEQFLEDQITAVRKGKIHPEKGTALYEFSHWEDMNGKPMDARLCAVDLMNVIRPLVAINKFVSFGVLALHEFPGERVRVALNEGDYAYKFVQEVRRYYPFVPFLPGKAKENITFNGYKIQKDTMMLLDIYGTLHRDDLFSEPERFNPYRFENWDGSPFDLIPQGGGDYYTNHRCAGEWMTIIIMEETMKFFANEISYDVPPQDFTVDTTNFPGKVASGMDIENIRVNIDRTK

SEQ ID NO: 9 - protein, 425 aa, Macrococcus caseolyticus
MSKRVPKDRGIDNSLKIMKEGYEYVPARMKKFNTNIFETRVLGGKTAVVISGKDAAELFYDNDKTERKGTLPKRVVKTLFGKGAIHTTTGKKHIDRKALFMSLMTDENLAYLRKLTRIYWFQNIEHMQYKQKVNVYEEATELLTKVGLRWAGITDNPDNIKKMADDMNKMIDSFSAIGSLYGGYREAKKARARVEQFLEDQITAVRKGKIHPEKGTALYEFSHWEDMNEKPMDARLCAVDLMNVIRPLVAINKFVSFGVLALHEFPGERVRVALNEEDYAYKFVQEVRRYYPFVPFLPGKAKENITFDGYKIQKDTMMILDIYGTLHRDDLFSEPERFNPYRFENWDGSPFDLIPQGGGDYYTNHRCAGEWMTIIIMEETMKFFANEISYDVPPQDFTVDTTKFPGKVASGMDIENIRVNIDRTQ

SEQ ID NO: 10 - protein, 425 aa, Macrococcus caseolyticus
MSKRIPKDRGIDNSFKIMKEGYEYVPARMKKFNTNIFETRVLGGKAAVVISGKDAAELFYDNDKTERKGTLPKRVVKTLFGKGAIHTTTGKKHIDRKALFMSLMTDENLAYLRKLTRIYWFQNIEHMQYKQKVNVYEEATELLTKVGLRWAGITDNPDNIKKMADDMNKMIDSFSAIGSLYGGYREAKKARARVEQFLEDQITAVRKGKIHPEKGTALYEFSHWEDMNGKPMDARLCAVDLMNVIRPLVAINKFVSFGVLALHEFPGERVRVALNEEDYAYKFVQEVRRYYPFVPFLPGKAKENITFDGYKIQKDTMMILDIYGTLHRDDLFSEPERFNPYRFENWDGSPFDLIPQGGGDYYTNHRCAGEWMTIIIMEETMKFFANEISYDVPPQDFTVDTTKFPGKVASGMDIENIRVNIDRTQ

SEQ ID NO: 11 - protein, 425 aa, Macrococcus canis
MSKRIPKDRGIDNSLKIMKEGYEYVPARMKKFNTNIFETHVLGGKTAVVISGKEAAELFYDNDKTERKGTLPKRVVKTLFGKGAIHTTAGKTHIDRKALFMSLMTDENLAYLRKLTRNYWFQNIEHMQYKQKVNVYEEATELLTKVGLRWAGIVDNPDNIKKMADDMNKMIDSFSAIGSLYGGYREAKKARARVEQFLEDQITAVRKGKIHPEKGTALYEFSHWEDMNGKPMDARLCAVDLMNVIRPLVAINKFVSFGVLALHEFPGERVRVALNEEDYAYKFVQEVRRYYPFVPFLPGKAKENITFDGYKIKKDTMMILDIYGTLHRDDLFSEPDRFNPYRFDNWDGSPFDLIPQGGGDYYTNHRCAGEWMTIIIMEETMKFFANEISYDVPPQDFTVDTTKFPGKVASGMDIENIRVNIDRTK

SEQ ID NO: 12 - protein, 425 aa, Macrococcus canis
MSKRIPKDRGIDNSLKIMKEGYEYVPARMKKFNTNIFETHVLGGKTAVVISGKEAAELFYDNDKTERKGTLPKRVVKTLFGKGAIHTTAGKTHIDRKALFMSLMTDENLAYLRKLTRNYWFQNIEHMQYKQKVNVYEEATELLTKVGLRWAGIVDNPDNIKKMADDMNKMIDSFSAIGSLYGGYREAKKARARVEQFLEDQITAVRKGKLHPEKGTALYEFSHWEDMNGKPMDARLCAVDLMNVIRPLVAINKFVSFGVLALHEFPGERVRVALNEEDYAYKFVQEVRRYYPFVPFLPGKAKENITFDGYKIKKDTLMILDIYGTLHRDDLFSEPERFNPYRFDNWDGSPFDLIPQGGGDYYTNHRCAGEWMTIIIMEETMKFFANEISYDVPPQDFTVDTTKFPGKVASGMDIENIRVNIDRTK

SEQ ID NO: 13 - protein, 425 aa, Macrococcus canis
MSKRIPKDRGIDNSLKIMKEGYEYVPARMKKFNTNIFETHVLGGKTAVVISGKEAAELFYDNDKTERKGTLPKRVVKTLFGKGAIHTTAGKTHIDRKALFMSLMTDENLAYLRKLTRNYWFQNIEHMQYKQKVNVYEEATELLTKVGLRWAGIVDNPDNIKKMADDMNKMIDSFSAIGSLYGGYREAKKARARVEQFLEDQITAVRKGKLHPEKGTALYEFSHWEDMNGKPMDARLCAVDLMNVIRPLVAINKFVSFGVLALHEFPGERVRVALNEEDYAYKFVQEVRRYYPFVPFLPGKAKENITFDGYKIKKDTLMILDIYGTLHRDDLFGEPERFNPYRFDNWDGSPFDLIPQGGGDYYTNHRCAGEWMTIIIMEETMKFFANEISYDVPPQDFTVDTTKFPGKVASGMDIENIRVNIDRTK

SEQ ID NO: 14 - protein, 425 aa, Macrococcus canis
MSKRIPKDRGIDNSLKIMKEGYEYVPARMKKFNTNIFETHVLGGKTAVVISGKEAAELFYDNNKTERKGTLPKRVVKTLFGKGAIHTTAGKTHIDRKALFMSLMTDENLAYLRKLTRNYWFQNIEHMQYKQKVNVYEEATELLTKVGLRWAGIVDNPDNIKKMADDMNKMIDSFSAIGSLYGGYREAKKARARVEQFLEDQITAVRKGKLHPEKGTALYEFSHWEDMNGKPMDARLCAVDLMNVIRPLVAINKFVSFGVLALHEFPGERVRVALNEEDYAYKFVQEVRRYYPFVPFLPGKAKENITFDGYKIKKDTLMILDIYGTLHRDDLFGEPERFNPYRFDNWDGSPFDLIPQGGGDYYTNHRCAGEWMTIIIMEETMKFFANEISYDVPPQDFTVDTTKFPGKVASGMDIENIRVNIDRTK

SEQ ID NO: 15 - protein, 425 aa, Macrococcus canis
MSKRIPKDRGIDNSLKIMKEGYEYVPARMKKFNTNIFETHVLGGKTAVVISGKEAAELFYDNDKTERKGTLPKRVVKTLFGKGAIHTTAGKTHIDRKALFMSLMTDENLAYLRKLTRNYWFQNIEHMQYKQKVNVYEEATELLTKVGLRWAGIVDNPDSIKKMADDMNKMIDSFSAIGSLYGGYREAKKARARVEQFLEDQITAVRKGKLHPEKGTALYEFSHWEDMNGKPMDARLCAVDLMNVIRPLVAINKFVSFGVLALHEFPGERVRVALNEEDYAYKFVQEVRRYYPFVPFLPGKAKENITFDGYKIKKDTLMILDIYGTLHRDDLFGEPERFNPYRFDNWDGSPFDLIPQGGGDYYTNHRCAGEWMTIIIMEETMKFFANEISYDVPPQDFTVDTTKFPGKVASGMDIENIRVNIDRTK

SEQ ID NO: 16 - protein, 425 aa, Macrococcus sp.
MAKRMPKDRGLDNTLTIMKEGYEYIQNRTKKFNSNVFETRVLGGKQAVVISGKQAAELFYDNDKIERSGTLPPRVVKTLFGKGAIHTTTGKKHIDRKALFMSLMTDENLTYLRKLTRNYWFQNTQRMQLMDEVNVYKESIILLTKVGFRWAGIIETPENIEQHAMDMDKMIDSFGAIGSPYKGYREAKKARARVEDFLEEQIIKTRKGKIHPEPGSALYEFSHWEDMNGKPMDSRLCSVDLMNVIRPLVAINRFVAYGVLAMHEFPGERTRVARDDNDYAYKFVQEVRRYYPFVPFLPGKAAKNIEFDGYKIKKDTMIILDVFGTLHREDTFSEPERFNPYRFDNWDGSPFDLIPQGGGDYYTNHRCAGEWMTIIIMEETMKYFAQEIDYDVPPQDFTVDRTKLPGRVKSGMVINNVRLNIDRTK

SEQ ID NO: 17 - protein, 425 aa, Macrococcus goetzii
MAKRMPKDRGLDNTLTIMKEGYEYIQNRTKKFNSNVFETRVLGGKQAVVISGKQAAELFYDNDKIERSGTLPPRVVKTLFGKGAIHTTTGKKHIDRKALFMSLMTDENLTYLRKLTRNYWFQNTQRMQLMDEVNVYKESIILLTKVGFRWAGIIETPENIEQHAMDMDKMIDSFGAIGSPYKGYREAKKARARVEDFLEEQIIKTRKGKIHPEPGSALYEFSHWEDMNGKPMDSRLCSVDLMNVIRPLVAINRFVAYGVLAMHEFPGERTRVARDDNDYAYKFVQEVRRYYPFVPFLPGKAAKNIEFDGYKIKKDTMIILDVFGTLHREDTFSEPERFNPYRFDNWDGSPFDLIPQGGGDYHTNHRCAGEWMTIIIMEETMKYFAQEINYDVPPQDFTVDRTKLPGRVKSGMIINNVRLNIDRTK

SEQ ID NO: 18 - protein, 425 aa, Macrococcus goetzii
MAKRMPKDRGLDNTLTIMKEGYEYIQNRTKKFNSNVFETRVLGGKQAVVISGKQAAELFYDNDKIERSGTLPPRVVKTLFGKGAIHTTTGKKHIDRKALFMSLMTDENLTYLRKLTRNYWFQNTQRMQLMDEVNVYKESIILLTKVGFRWAGIIETPENIEQHAMDMDKMIDSFGAIGSPYKGYREAKKARARVEDFLEEQIIKTRKGKIHPEPGSALYEFSHWEDMNGKPMDSRLCSVDLMNVIRPLVAINRFVAYGVLAMHEFPGERTRVARDDDDYAYKFVQEVRRYYPFVPFLPGKAAKNIEFDGYKIKKDTMIILDVFGTLHREDTFSEPERFNPYRFDNWDGSPFDLIPQGGGDYHTNHRCAGEWMTIIIMEETMKYFAQEIDYDVPPQDFTVDRTKLPGRVKSGMIINNVRLNIDRTK

SEQ ID NO: 19 - protein, 425 aa, Macrococcus epidermidis
MAKRMPKDRGLDNTLTIMKEGYEYIQNRTKKFNSNVFETRVLGGKQAVVISGKQAAELFYDNDKIERSGTLPPRVVKTLFGKGAIHTTTGKKHIDRKALFMSLMTDENLTYLRKLTRNYWFQNTQRMQLMDEVNIYKESIILLTKVGFRWAGIIETPENIEQHAMDMDKMIDSFGAIGSPYKGYREAKKARARVEDFLEEQIIKTRKGKIHPEPGSALYEFSHWEDMNGKPMDSRLCSVDLMNVIRPLVAINRFVAYGVLAMHEFPGERTRVARNDNDYAYKFVQEVRRYYPFVPFLPGKAAKNIEFDGYKIKKDTMIILDVFGTLHREDTFSEPERFNPYRFDNWDGSPFDLIPQGGGDYHTNHRCAGEWMTIIIMEETMKYFAQEINYDVPPQDFTVDRTKLPGRVKSGMIINNVRLNIDRTK

SEQ ID NO: 20 - protein, 425 aa, Macrococcus goetzii
MAKRMPKDRGLDNTLTIMKEGYEYIQNRTKKFNSNVFETRVLGGKQAVVISGKQAAELFYDNDKIERSGTLPPRVVKTLFGKGAIHTTTGKKHIDRKALFMSLMTDENLTYLRKLTRNYWFQNTQRMQLMDEVNVYKESIILLTKIGFRWAGIIETPENIEQHAMDMDKMIDSFGAIGSPYKGYREAKKARTRVEDFLEEQIIKTRKGKIHPEPGSALYEFSHWEDMNGQPMDSRLCSVDLMNVIRPLVAINRFVAYGVLAMHEFPGERTRVARDDDDYAYKFVQEVRRYYPFVPFLPGKAAKNIEFDGYKIKKDTMIILDVFGTLHREDTFSEPERFNPYRFDNWDGSPFDLIPQGGGDYHTNHRCAGEWMTIIIMEETMKYFAQEIDYDVPPQDFTVDRTKLPGRVKSGMIINNVRLNIDRTK

SEQ ID NO: 21 - protein, 425 aa, Macrococcus lamae
MSKPIPVDKGIDKTLTVMKEGYEYVSNRTKKFNSDIFETRVLGGKRAMVISGKEAAELFYDNDKIERKGTLPPRVVKTLFGKGAIHTTTGKVHIDRKSLFMSLMTENNLEYLRKLTRNFWYQNTERMQLMGKVNVYKESIILLTKVGFRWAGVTEKPEKIEQHAMDMDKMIDSFGSVGTAYKGYREAKKARARVEDFLEKQIIATRKGKIHPEEGTALHSFSHWRDFQGNYMDSRLAAVDLMNVIRPLVAINRFVAYGVLALHEFPGERERVSADENNYAYKFTQELRRYFPFVPFLPAKAKVDIDFQGHVIPQDTMIVLDIFGTLHREDLWEDPERFYPDRFENWDGSPFDLIPQGGGDYHTNHRCAGEWMTIIIMEETMKYFAREITYDVPAQDFTVDRTKLPGRVKSGMDIENVRLNFDRTL

SEQ ID NO: 22 - protein, 426 aa, Macrococcus bovicus
MAKRMPKVKGLDQTLTVLKEGYEFIPNRTRKLNTNVFEIRILGGKKAVVISGKEAAELFYDNDKTERAGTLPKRLVKTLFGKGAIHTTTGDKHKNRKSLFMSLMTDNNLEYLRKLTRNYWFEDTARLQAMDQTNVYQEATLVLLKIGFRFADVPFDTPERMEQYAKDMNAMVDSFGSIGTAYTGYRRALNARDRVEDYLEKQIIATRKGKLFPEEGSGLYEFSHWKDMNGKHMDSRLAAVDLMNTIRPLVAINRFISFGVLAMHEYPGERARVAQNDNSYAYKFAQEVRRYYPFVPFLPGKAKQNITFEGYKIDKDTMLILDIFGTLHSESLWENAEQFYPERFENWDGSPFDLIPQGGGDYYTNHRCAGEWMTIIIMEETMKYFAREIQYDVPPQDFTVDRTKLPGQINSGMIIENIRNNFDRRR

SEQ ID NO: 23 - protein, 426 aa, Macrococcus hajekii
MAKRIPKVKGFDQTLNVIKEGYEFIPNRTKKFNSNVFEMRVLGGKRAIVISGKEAAELFYDNEKIERQGTLPKRIVKTLFGKGAIHTTTGAKHKDRKSLFMSLMTENNLEYLRKLTRNYWFEDTARLQSKEQVNVFEESTLVLLKVAFRFADVPFDTPERMEQFAKDMNSMVDSFGSIGTVYSGYKKARDARTRVEDYLEKQIKLTRKGKLFPEEGSGLYEFSHWKDMNGKQMDSRLAAIDLMNTIRPLVAINRFISFGVLAMHEFPGERVKVAEDDNSYAYKFVQEIRRYYPFVPFLPGKVKQNFTFDGYKFDKETMVIMDIFGTLHSESLWNNAEQFYPERFENWDGSPFDLIPQGGGDYYTNHRCAGEWMTIIIMEETMKYFAREIQYDVPAQDFTVNRQKLPGQVKSGMIIENIRNNFDRRR

SEQ ID NO: 24 - protein, 426 aa, Macrococcus sp.
MSKSIPKEQGLDQTLNILKEGYEFIPNRTKKFNSNVFEIRVLGGKRAIVISGKEAAELFYDNEKIERAGTLPKRLVNTLFGKGAIHTTTGATHKDRKSLFMSLMTENNLEYLRKLTRNYWYTDTERLQMKEQTNVYLESTLVLLKIGFRFADVPFDTPERMEQYAKDMNAMVNSFGSIGTVYSGYKKALDARSRVEDYLEKQIKLTRKGKLNPEAGSGLYEFSHWKDMNGNHMDARLAAVDLMNTIRPLVAINRFISFGVLAMHEYPGERAKVAKDENSYAYKFAQEVRRYYPFVPFLPGKAKTDFTFEGYKFDKETMIILDIFGTLHSESLWNNAEQFYPERFEHWDGSPFDLIPQGGGDYYTNHRCAGEWMTIIIMEETMKYFAREIQYDVPAQDFTVDRTKLPGQVKSGMIIENIRNNFDRRQ

SEQ ID NO: 25 - protein, 423 aa, Salinicoccus luteus
MSTIKKHKGLDNTLKVMKEGYLYTTNQRRRLGSDNIFETRVLGGKRAIVISGKKAAELFYDNDKTERSGTLPKRLVNTLFGKGAIHTTTGKKHVDRKALFMSLMTEGNLKHLRELTRNHWYMNTHRMEQMDQINIYRESIILLTKVGTKWAGVQAPEEKIEEIATDMDIMIDSFKGLGTAYKGYKASVAARRRVEDWLEDQIIQTRKGKIFPPEGTALYEFAHWEDYKGNPMDSRLCAIDLMNTFRPLIAINRFVSFGLLAMYEHPISKEKIKNEPDYAYMFSQEVRRFYPFVPFLPGKAKTDIQFEGHDIEKDTMLVLDIYGTMHRDDVFENADEFYPERFRDWDGSPFDLIPQGGGDYYTNHRCAGEWMTIIIMEETMKYFAGKISYDVPEQDLTVDLNSIPGYIKSGFIIENVQENVDRG

SEQ ID NO: 26 - protein, 423 aa, Salinicoccus roseus
MSTIKKHKGLDNTLKVMKEGYLYTTNQRRRLGAENIFETRVLGGKRALVISGKKAAELFYDNDKTERSGTLPKRLVNTLFGKGAIHTTTGKKHIDRKALFMSLMTEGNLKHLRELTRNHWYMNTHRMEQMDQINIYRESIILLTKVGTKWAGVQAPEEKIEEIATDMDIMIDSFRGLGTAFKGYKESVAARRRVEDWLEDQIIQTRKGKIFPPEGTALYEFAHWEDYKGNPMDSRLCAIDLMNTFRPLIAINRFVSFGLLAMHEHPISKEKINNDPDYAYMFSQEVRRFYPFVPFLPGKAKTDIQFEGHDIEKDTMLVLDIYGTMHRDDVFENADEFHPERFLDWDGSPFDLIPQGGGDYYTNHRCAGEWMTIIIMEETMKYFAGKITYDVPEQDLTVDLNSIPGYIKSGFIIENVAEKVDRK

SEQ ID NO: 27 - protein, 423 aa, Salinicoccus roseus
MSTIKKHKGLDNTLKVMKEGYLYTTNQRRRLGAENIFETRVLGGKRALVISGKKAAELFYDNDKTERSGTLPKRLVNTLFGKGAIHTTTGKKHIDRKALFMSLMTEGNLKHLRELTRNHWYMNTHRMEQMDQINIYRESIILLTKVGTKWAGVQAPEEKIEEIATDMDIMIDSFRGLGTAFKGYKASVAARRRVEDWLEDQIIQTRKGKIFPPEGTALYEFAHWEDYKGNPMDSRLCAIDLMNTFRPLIAINRFVSFGLLAMHEHPISKEKINNDPDYAYMFSQEVRRFYPFVPFLPGKAKTDIQFEGHDIEKDTMMVLDIYGTMHRDDVFENADEFYPERFLDWDGSPFDLIPQGGGDYYTNHRCAGEWMTIIIMEETMKYFAGKITYDVPEQDLTVDLNSIPGYIKSGFIIENVAEKVDRK

SEQ ID NO: 28 - protein, 423 aa, Salinicoccus albus
MSTIKRDTGLDNTLKVMKQGYLYTENQRQRLGSDNIFETRVLGGKRALVISGKRAAELFYDNDKIERAGTLPKRLVNTLFGKGAIHTTTGKQHIDRKALFMSLMTEGNLKHLRELTRNHWYMNTHRMEQMDQINIYRESIILLTKVGTKWAGVQAPEEKVEEIATDMDIMIDSFRGLGTAFKGYKKSKDARRRVEDWLEDQIIQTRKGKIFPPEGTALYEFAHWEDYKGNPMDSRLCAIDLMNTFRPLIAINRFVSFGLLAMHEHPISKEKIKSDDDYAYMFSQEVRRYYPFVPFLPGKAKTDIEFDGHAIEKDTMMVIDIYGTMHRDDVFENANEFYPERFKEWDGSPFDLIPQGGGDYYTNHRCAGEWMTIIIMEETMKYFADRISYDVPQQDLTVDLNSIPGYIKSGFVIENVSENVDRN

SEQ ID NO: 29 - protein, 435 aa, Jeotgalibacillus alimentarius
MDVKKPVPRAKGIDNSLNFIKEGFHFLPNQRQELGSDIFETRLLGKKAVCIAGVEASEMFYDTEKFKREGAMPKPFKMSLFGEGSLHGMDGEAHLNRKRTLLSMFTPDRVEDLKRMAIKHLDEKVSEWEHADDVVIFDEMQEILARAGCEWAGVPLEEEEVKQRTREMIDMIDSFGGSLSRFKDGKKARNSHEAWLQDIIKGVRKGKMSPKPNTAAYIMSHHRENNGKRFDKEIAAVELSNAFRPLLATAYFLTFGVLAMHEHPETVEKLKNNAKYSHMFAQETRRYYPFVPAMVAKVKKNFTFKGYKFKKDTLVVLDIFGTNRHPDSWENADTFIPERFDGWKGSPFSFIPQGGGDHHTGHRCAGEWLTVIVMSSFFDFFAKNLTYDVPEQDLSFSMERMPTFPNSRLVIKNVRKTDDYEKNLAELMDGFSAIR

SEQ ID NO: 30 - protein, 436 aa, Jeotgalibacillus campisalis
MKVKTPIPRAKGIDNSLSLIKEGFHFLPNQRKEMQSDIFKTRILGQKAICIAGEEAAEVFYNNDLFKRKGAMPKPLKLSLFGEGSLHGLDGEPHKNRKRMFLSMFTPERVEDLVDLALKHLDAKAGEWQSQDGVAVLDQMQEILTRAGCEWAGVPLAEGEVQQRTDEVVAMVDSFGGSLSRFRDGKKARDSHEEWLQKIIKSVRKGKYNPPENTAVYTIAHHRDEKGKPFDLHTAAVELSNAFRPLIATANFLVFGALAMHEHPETRQKIAADEDRYSHMFSQEVRRYYPFVPAMAAKVKKDFTWHDYTFKKDTLVILDIFGTNRHPDSWEQAETFLPERFKDWQGSPFAFIPQGGGDHHIGHRCAGEWLTVYVMRAFFKYFAQNISYDVPEQDLSYSLERMPTFPKSGFVITNVKKAADSDELLHSMQKRHTAIQ

SEQ ID NO: 31 - protein, 435 aa, Jeotgalibacillus malaysiensis
MDVKKPVPRSKGIDNSLSLIKEGFYFLPNQRQKLGSDIFETRLLGKKAICMAGEEASELFYDTEKFKREDAMPKPLKMSLFGDGSLHGMDGEAHRNRKRSFLSMFTPDRVEDLKRLALEHLDAKVSHWEKADEVVIFDEMQEILARAGCEWAGVPLEENEVQQRTQEMIDMIDSFGGSLSRFKDGKKARDSHEAWLQEIIKGVRKGKMSPPPHTAAYVMAHHRENNGKRIDKGTAAVELSNSFRPLLATAYFLTFGIVAMHEHPLTVSKLQHHPRYSHMFAQETRRFYPFVPAMAAKVKKNFTFKGYKFKKDTLVVLDIFGTNRHPDSWENADTFIPERFEGWKGSPFSFIPQGGGDHHTGHRCAGEWLTVIVMSSFFEFFAKNLTYDVPEQDLSYSMERMPTFPNSRMIIQNVRKIADYEKNLNELMDGFSAIR

SEQ ID NO: 32 - protein, 436 aa, Jeotgalibacillus proteolyticus
MKVKTPIPRAKGIDNSLSLIKEGFHFLPNQRKEVESDIFKTRILGQKAICMCGEEAAEVFYDPDKFKRKGALPKPLKMSLFGDGSLHALDGEDHRNRKRMFLSMFTPERVEDLKKIALEHLAAKVDVWEKSDQVIFFGEMQEILTRAGCEWAGFPLEEEEVHQRTEEIVAMIDSFGGSLSRFRDGKKARDSHEEWLEGIIKAVRKGKMNPPAYTAAYIVAHHRENNGKRLDVRTAAIELSNAFRPLIATAYFLTFGAAAMHEHPETAHKLMENKDHYSHMFSQEVRRYYPFVPAMAAKVKEDFVWQDHKFKKNTLVILDIFGTNRHPDLWSDADSFIPERFRDWKGSPFSFIPQGGGDHHIGHRCAGEWLTVYVMRSFFQFLAQNITYDVPEQDLSYSLERMPTFPVSGLVITNVRKQADFNEKLNELDKSYTAIR

SEQ ID NO: 33 - protein, 434 aa, Halobacillus salinus
MSKPIPKAEGADNTLSLIQEGFPFFIKRHKELGTNIFETRLIGRKAICIVGEEASKIFYDTEKFKREGAMPKPLKMSLFGEGSLHGMDGEKHLHRKRTFLSMFTPDRVEDLKQNALEELDRKMEQWQERDKVVLYEEFPEILTRAVCKWAGVPLEEHEVRQRTDEMMSMVESFGGSLSRFQEGKKSRDSHEAWLKQIIKDVRKGKLQPEPNTAAYIMAHHRETDGKLWDVQTAAVELSNSFRPLLATVHLLVLGAVAMHEHPETRTRLEKGEPLYSHWFAQETRRFYPFVPAMIAKVKTPFLWEGYEFKKNTRVVLDLYGTNHHADSWVHPDAFIPERFENWQGSPFSFVPQGGGDHHTGHRCAGEWLTVIVMSSFFEFLAKNVSYDVPEQNLSFSLERMPTRPESGFVIENVRKKDAYEETFDELNQGFAARR

SEQ ID NO: 34 - protein, 436 aa, Bacillus clarkii
MKVKTPIPKAKGIDNSMSLIKEGFHFLPNRREELGSDIFETRLMGRKAVCMSGEEAAEVFYDNDKFKRKGALPKPLQLSLFGTGSVHALDGEAHKNRKRMFLSMFTPERVEDLNRIALKHLDNKVNQWVKRDKVILIDEMHEILTRAGCEWAGVPLKEEEVEQRTAEVGQMIDSFGGSLSRWRNGKKARDSHEEWLEGIIKKIRKGKMEVPANTAAYIVAHHRELNGKRLDSRQAAIELSNSFRPLIATAYFLTLGAVAMHEHPGTMEKLKKDEGNYSHMFSQEVRRYYPFVPAMAAKVKKDFVWHDYKFKKDTLVVLDIFGTNRHPDNWEQADEFIPERFKDWKGSPFSFIPQGGGDHHIGHRCAGEWLTVMVMRSFFKYFVNNMNYHVPEQNLAYSLERMPTFPKSGFVITDVKKASDSGEKMQEMMHNPITVK

SEQ ID NO: 35 - protein, 436 aa, Bacillus polygoni
MKVKTPIPKAKGIDNSMSLIKEGFHFLPNRREELGSDIFETRLMGRKAVCMSGEEAAEVFYDNDKFKRKGALPKPLQLSLFGTGSVHALDGEAHKNRKRMFLSMFTPERVEDLNRIALKHLDNKVNQWVKRDKVILIDEMHEILTRAGCEWAGVPLKEEEVEQRTAEVGQMIDSFGGSLSRWRNGKKARDSHEEWLEGIIKKIRKGKMEVPANTAAYIVAHHRELNGKRLDSRQAAIELSNSFRPLIATAYFLTLGAVAMHEHPGTMEKLKKDEGNYSHMFSQEVRRYYPFVPAMAAKVKKDFVWHDYKFKKDTLVVLDIFGTNRHPDNWEQADEFIPERFKDWKGSPFSFIPQGGGDHHIGHRCAGEWLTVMVMRSFFKYFVNNMTYHVPEQNLAYSLERMPTFPKSGFVITDVKKAADSGEKMQEMMHNPITVK

SEQ ID NO: 36 - protein, 434 aa, Halobacillus salinus
MSKPIPKAEGADNTRSLIQEGFPFFIKRHKELGTNIFETRLIGRKAICIVGEEASKIFYDTEKFKREGAMPKPLKMSLFGEGSLHGMDGEKHLHRKRTFLSMFTPDRVEDLKQNALEELDRKVEQWQERDKVVLYEEFPEILTRAVCKWAGVPLEEHEVRQRTDEMMSMVESFGGSLSRFQEGKKSRDSHEAWLKQIIKDVRKGKLQPEPNTAAYIMAHHRETDGKLWDVQTAAVELSNSFRPLLATVHLLVLGAVAMHEHPETRTRLEKGEPLYSHWFAQETRRFYPFVPAMIAKVKTPFLWEGYEFKKNTRVVLDLYGTNHHADSWVHPDAFIPERFENWQGSPFSFVPQGGGDHHTGHRCAGEWLTVIVMSSFFEFLAKNVSYDVSEQNLSFSLERMPTRPESGFVIENVRKKDAYEETFDELNQGFAARR

SEQ ID NO: 37 - protein, 435 aa, Halobacillus sp.
MIHKDIPRAKGIDNTMNLIKEGFHFLPNQRQALNSDVFETRLIGKKAICIAGEEASKLFYDTEKFKREGAMPKPLKMSLFGDGSLHGLDGEEHRNRKRTFLSMFTPDRVEDLKKLALKHLDAKATEWQEQEEVYLFDEMQEILARAGCEWAGVPLQEEEVKQRTDEMIEMIDSFGGSLSRFREGKQARNHHEKWLGQIIKDVRKGKLDPEPNTAAYIMAHHRETNGKKWDTRTAAVELSNSFRPLLATAYFLTFGAVAMHEHPESKEKLKQDTSRYSHWFAQETRRYYPFVPAMVAKVKEDFIWNDFLFKKNTLVVLDIFGTNRHPDSWEEPDAFIPERFKDWEGSPFSFIPQGGGDHHTGHRCAGEWLTVIVMSSFFEFFAKNLSYDVPEQDLSFSMERMPSYPNSRFKIQNVKKTGYYDEHLSDLNRGFSAIR

SEQ ID NO: 38 - protein, 438 aa, Planomicrobium flavidum
MRIRKPIPKAEGLDNTLALIKEGFHFIPSRREKLASDIFETRLIGRKAVCMAGEEAAAIFYDNNLFKRAGAAPKPLKQGLLGVGGIHGRDGEDHHQQKRMFLSMMTPERLEDMRRIAVEELDRKAAAWQKEDSVAMLPEMEEIMGLAGMRWAGLPLGSEQEAKRRAKELADMVDSFGGSFSRYQEGKRARESHEEWLRNIIRDIRQEKYNPPPYTAAYIVAHHRGADGKLLDLETAAVDLNNSYRPLVATAYFIVFGMLAMYEHPETVAKIRSNEGNYSHMFSQEIRRYYPFAPAMVAKAKKDFIWHGYHFKKDQTVVLDLFGTNRHPDNWEEADRFIPERFHNWSGSPFAFVPQGGGDHYMGHRCAGEWMTVIVMQAFFHYLTAGVAYDVPEQDLTYDLRRMPTMPKSGFVIRNVRRSGNYPLDALMEDQNHTAVKF

SEQ ID NO: 39 - protein, 422 aa, Staphylococcus delphini
MAKKLPKDTGLDNTLKIINEAYTYVPKRLEKFGTKAFETRALGMKPVVVISGKAAAELFYDNDKISRKGTLPKRIVHTLFGKGAIHTTEGKVHVDRKALFMSLMTEKNLKYLRELTRNYWFMHTERMQNKDKVNVYQEAGLILTKVGFRWAGLKQTDEQAAQNAEDMNTMIDSFSGLGQSLKGYREAKKARARVEQFLQDQIEAVREGQQYAEPGTALYEFAHWKDLNDQPMDPYLCAVDLMNIVRPLVAVNRFVSYGVKALIEFDQERKKLQVTNDPNYAYKFAQEVRRIFPFVPFLPGRLKKTVEFDGFKLKKGTLTVLDIFGTTHDPELFENPYQFNPDRFDNWDGSPFDLIPQGGGDFYTNHRCAGEWMTVIVMEETIQYFANKIDFEAPAQDLSVKLDQFPGKVTSGTIIKSVYPRI

SEQ ID NO: 40 - protein, 422 aa, Staphylococcus delphini
MAKKLPKDTGLDNTLKIINEAYTYVPKRLEKFGTKAFETRALGMKPVVVISGKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLKQTDEQAAQNAEDMNTMIDSFSGLGQSLKGYREAKKARARVEQFLQDQIEAVREGQQYAEPGTALYEFAHWKDLNDQPMDPHLCAVDLMNIVRPLVAVNRFVSYGVKALIEFDQERKKLQVTNDPNYAYKFAQEVRRIFPFVPFLPGRLKKTVEFDGFKLKKGTLTVLDIFGTTHDPELFENPYQFNPDRFDNWDGSPFDLIPQGGGDFYTNHRCAGEWMTVIVMEETIQYFANKIDFEAPAQDLSVKLDQFPGKVTSGTIIKSVYPRI

SEQ ID NO: 41 - protein, 422 aa, Staphylococcus delphini
MAKKLPKDTGLDNTLKIINDAYTYVPKRLEKFGTKAFETRALGMKPVVVISGKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLKQTDEQAAQNAEDMNTMIDSFSGLGQSLKGYREAKKARARVEQFLQDQIEAVREGQQYAEPGTALYEFAHWKDLNDQPMDPHLCAVDLMNIIRPLVAVNRFVSYGVKALIEFDQERKKLQVTNDPNYAYKFAQEVRRIFPFVPFLPGRLKKTVEFDGFKLKKGTLTVLDIFGTTHDPELFENPYQFNPDRFDNWDGSPFDLIPQGGGDFYTNHRCAGEWMTVIVMEETIQYFANKIDFEAPAQDLSVKLDQFPGKVTSGTIIKSVYPRI

SEQ ID NO: 42 - protein, 423 aa, Salinicoccus sp. CT19
MSTIKRDKGLDNTLKVMKEGYLYTTNQRNRLGADNIFETRGLGGKRVLVLSGKKAAETFYDNDKIERKGTLPKRVVNTLFGKGAIHTTTGKKHIDRKALFMSLMTEGNLKHLRELTRNHWYMNTHRMEQMDQINIYRESIILLTKVGTKWAGVQAPEEKIEEIATDMDIMIDSFRGLGTAFKGYRSSVKARRRVEDWLEDQIIQTRKGKIFPPEGTALYEFAHWEDYLGNPMDSRLCAIDLMNTFRPLIAINRFVSFGLLAMHEFPVSREKINNEDDYAYMFSQEVRRFYPFVPFLPAKAKTDFHFDGHKVDKDTMLLLDIYGTMHREDVFENPNEFRPERFLDWDGSPFDLIPQGGGDYYTNHRCAGEWMTVIIMEETMKYFAGRITYDVPEQDLRVDLNSIPGYVKSGFVIENVKENLDRR

SEQ ID NO: 43 - protein, 422 aa, Staphylococcus delphini
MAKKLPKDTGLDNTLKIINEAYTYVPKRLEKFGTKAFETRALGMKPVVVISGKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLKQTDEQAAQNAEDMNTMIDSFSGLGQSLKGYREAKKARARVEQFLQDQIEAVREGQQYAEPGTALYEFAHWKDLNDQPMDPHLCAVDLMNIVRPLVAVNRFVSYGVKALIEFDQERKKLQVTNDPNYAYKFAQEVRRIFPFVPFVPGRLKKTVEFDGFKLKKGTLTVLDIFGTTHDPELFENPYQFNPDRFDNWDGSPFDLIPQGGGDFYTNHRCAGEWMTVIVMEETIQYFANKIDFEAPAQDLSVKLDQFPGKVTSGTIIKSVYPRI

SEQ ID NO: 44 - protein, 422 aa, Staphylococcus delphini
MAKKLPKDTGLDNTLKIINEAYTYVPKRLEKFGTKAFETRALGMKPVVVISGKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLKQTDEQAAQNAEDMNTMIDSFSGLGQSLKGYREAKKARARVEQFLQDQIEAVRAGQQYAEPGTALYEFAHWKDLNDQPMDPHLCAVDLMNIVRPLVAVNRFVSYGVKALIEFDQERKKLQVTHDPNYAYKFAQEVRRIFPFVPFLPGRLKQTVEFDGFKLKKGTLTVLDIFGTTHDPELFENPYQFNPDRFDNWDGSPFDLIPQGGGDFYTNHRCAGEWMTVIVMEETIQYFANKIDFEAPAQDLSVKLDQFPGKVTSGTIIKSVYPRI

SEQ ID NO: 45 - protein, 422 aa, Staphylococcus delphini
MAKQLPKDTGLDNTLKIINEAYTYVPKRLEKFGTKAFETRALGMKPVVVISGKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLKQTDEQAAQNAEDMNTMIDSFSGLGQSLKGYREAKKARARVEQFLQDQIEAVRAGQQYAEPGTALYEFAHWKDLNDQPMDPHLCAVDLMNIVRPLVAVNRFVSYGVKALIEFDQERKKLQVTHDPNYAYKFAQEVRRIFPFVPFLPGRLKKTVEFDGFKLKKGTLTVLDIFGTTHDPELFKNPYQFNPDRFDNWDGSPFDLIPQGGGDFYTNHRCAGEWMTVIVMEETIQYFANKIDFEAPAQDLSVKLDQFPGKVTSGTIIKSVYPRI

SEQ ID NO: 46 - protein, 422 aa, Staphylococcus delphini
MAKKLPKDTGLDNTLKIINEAYTYVPKRLENFGTKAFETRALGMKPVVVISGKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLKQTDEQAAQNAEDMNTMIDSFSGLGQSLKGYREAKKARARVEQFLQDQIEAVREGQQYAEPGTALYEFAHWKDLNDQPMDPHLCAVDLMNIVRPLVAVNRFVSYGVKALIEFDQERKKLQVTNDPNYAYKFAQEVRRIFPFVPFLPGRLKKTVEFDGFKLKKGTLTVLDIFGTTHDPELFENPYQFNPDRFDNWDGSPFDLIPQGGGDFYTNHRCAGEWMTVIVMEETIQYFANKIDFEAPAQDLSVKLDQFPGKVTSGTIIKSVYPRI

SEQ ID NO: 47 - protein, 422 aa, Staphylococcus delphini
MAKKLPRDTGLDNTLKIINEAYTYVPKRLEKFGTKAFETRALGMKPVVVISGKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLKQTDEQAAQNAEDMNLMIDSFSGLGQSLKGYREAKKARARVEQFLQDQIEAVRAGQQYAEPGTALYEFAHWKDLNDQPMDPHLCAVDLMNIVRPLVAVNRFVSYGVKALIEFDQERKKLQVTNDPNYAYKFAQEVRRIFPFVPFLPGRLKQTVEFDGFKLKKGTLTVLDIFGTTHDPELFENPYQFNPDRFDNWDGSPFDLIPQGGGDFYTNHRCAGEWMTVIVMEETIQYFANKIDFEAPVQDLSVKLDQFPGKVTSGTIIKSVYPRI

SEQ ID NO: 48 - protein, 422 aa, Staphylococcus cornubiensis
MAKKLPKDTGLDNTLKIINEAYTYVPKRLEQFGTKAFETRALGMKPVVVISGKEAAKLFYDNDKISRKGTLPKRIVHTLFGKGAIHTTEGKVHVDRKALFMSLMTEKNLKYLRELTRNYWFMNTERMQNMDEVNVYQEAGLILTQVGFRWAGLKQTDEEAAKNAEDMNIMIDSFSGLGQSLKGYREAKKARARVEQFLQDQIEAVRAGEQYAEPGTALYEFAHWKDLNDQPMDPHLCVVDLMNIVRPLVAVNRFVSYGVKALIEFDQERKKLQVTNDANYAYKFAQEVRRIFPFVPFLPGRLTKTVEFEGFKIKKDTLVALDIFGTTHDPELFENPYQFNPDRFENWDGSPFDLIPQGGGDFYTNHRCAGEWMTVIVMEETMQYFANKIDFDAPAQDLSVKLDQFPGKVTSGTIIKNVYPRI

SEQ ID NO: 49 - protein, 422 aa, Staphylococcus delphini
MAKKLPRDTGLDNTLKIINEAYTYVPKRLEKFGTKAFETRALGMKPVVVISGKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLKQTDEQAAQNAEDMNTMIDSFSGLGQSLKGYREAKKARARVEQFLQDQIEAVRAGQQYAEPGTALYEFAHWKDLNDQPMDPHLCAVDLMNIVRPLVAVNRFVSYGVKALIEFDQERKKLQVTHDPNYAYKFAQEVRRIFPFVPFLPGRLKQTVEFDGFKLKKGTLTVLDIFGTTHDPELFENPYQFNPDRFDNWDGSPFDLIPQGGGDFYTNHRCAGEWMTVIVMEETIQYFANKIDFEAPAQDLSVKLDQFPGKVTSGTIIKSVYPRI

SEQ ID NO: 50 - protein, 422 aa, Staphylococcus intermedius
MAKKLPKDTGLDNTFKIINEAYTYVPKRLEQFGTKAFETRALGMKPVVVISGKAAAKLFYDNDKISRKGTLPKRIVHTLFGKGAIHTTEGKVHVDRKALFMSLMTEKNLKYLRELTRNYWFMHTERMQNMDEVNVYKEAGLILTQVGFRWAGLKQTDEEAAKNAEDMNIMIDSFSGLGQSLKGYRQAKKARARVEQFLQDQIEAVRAGKQYAEPGTALYEFAHWKDLNNQPMDSHLCAVDLMNIVRPLVAVNRFVSYGVKALIEFDQERKKLQVTNDPNYAYKFAQEVRRIFPFVPFLPGRLTKTVEFDGFKIKKGTLVALDIFGTTHDPELFENPYQFNPDRFENWDGSPFDLIPQGGGDFYTNHRCAGEWMTVIVMEETIQYFANKIDFDAPAQDLSVKLDQFPGKVTSGTVIKNVYPRI

SEQ ID NO: 51 - protein, 423 aa, Salinicoccus roseus
MSTIKKHKGLDNTLKVMKEGYLYTTNQRRRLGAENIFETRALGGKRVLVISGKKAAELFYDNDKTERSGTLPKRLVNTLFGKGAIHTTTGKKHIDRKALFMSLMTEGNLKHLRELTRNHWYMNTHRMEQMDQINIYRESIILLTKVGTKWAGVQAPEEKIEEIATDMDIMIDSFKGLGTAFKGYKESVAARRRVEDWLEDQIIQTRKGKIFPPEGTALYEFAHWEDYKGNPMDSRLCAIDLMNTFRPLIAINRFVSFGLLAMHEYPISKEKINNDPDYAYMFSQEVRRFYPFVPFLPGKAKTDIQFEGHDIEKDTMLVLDIYGTMHQDDVFENADEFYPERFLDWDGSPFDLIPQGGGDYYTNHRCAGEWMTIIIMEETMKYFAGKITYDVPEQDLTVDLNSIPGYIKSGFIIENVAEKVDRK

SEQ ID NO: 52 - protein, 423 aa, Salinicoccus halodurans
MGTIKKDKGLDNTLKVMKQGYLYTANQRQRLGADNIFETRALGGKRVVVINGKEAAELFYDNDKTERSGTLPKRLVNTLFGKGAIHTTTGKKHIDRKALFMSLMTENNLQHLRELTRNHWYMNTHKMEQMDQINIYRESIVQLTKIGTKWAGVQAPEEKIEDIATDMDIMIDSFKGLGSAFKGYKESVNARKRVEDWLEEQIIETRKGKIFPPEGTALYEFAHWEDYQGNPMDSRLCAIDLMNTFRPLIAINRFVSFGLLAMHEHPISKEKIKTDDDYAYMFSQEVRRFYPFVPFLPGKAKTDIEFKGHKIEKDTMMVIDIYGTMHDEKVFENPDEFYPERFKEWDGSPFDLIPQGGGDYYTNHRCAGEWMTIIIMEETMKYFAERITYDVPEQDLTVDLNSIPGYVNSGFIIENVQENIDRK

SEQ ID NO: 53 - protein, 424 aa, Salinicoccus kekensis
MATIKKDKGIDNTAKIMKQGYLYTTNQRERLGVKDGVFETRALGGKRVIILSGKDGAELFYDNDKIERSGTLPKRVVNTLFGKGAIHTTTGKVHIDRKALFMSLMTEGNLQYLRELTRNHWQMNTQRMENMDQVNVYRESIILLTKVGTRWAGVQAPEKEIENIATDMDIMIDSFKAIGSAFKGYRASKAARRRVEDWLEDQIIQTRKGKIHPPEGTALYEFAHWKDYKGEPMDSRLCGIDLMNTFRPLIAINRFIAFGALAMYENPVAREKIKQDDDYAYMFAQEVRRFYPFVPFLPGKVKEDFQYKGYDFEKDTMLAIDIYGTMHDPTVWENPNEFYPERFKDWDGSPFDLIPQGGGDYYTNHRCAGEWMTVIIMQETMKYFASRITYDVPEQDLTVDLNSLPGYVKSGFIIENVREVVDRG

SEQ ID NO: 54 - protein, 421 aa, Staphylococcus felis
MNKIPKDKGLDNTLKIVKEAYTYVPRRLEAFDTNVFETRALGMKKVLVVSGKKAAELFYDNEKISRKGALPKRIVNTLFGQGAIHTTEGKQHVDRKALFMSLMTTKNLKYLRELTRNYWFAHTQRMEDMDEVNVYEEATLLLTKIGFRWAGLKQTHEEAVQNAKDMDLLIDSFSALGQSPAGYKKAKKARERVEKFLERQIKAVREGKQFAAPNTALYEFAHWKDLNDKPMDLHLCAVDLMNVVRPLVAVNRFVSYAVKALIEFDQERLKLQVTKDQNYAYKFAQEVRRIFPFVPFLPGRLKQNIEFDGYSLKKNTLIILDVFGTTHDPKLFENPYQFNPNRFEDWDGSPFDLIPQGGGDFYTNHRCAGEWMTVIVMEETINYFANKIDFSVPTQDLSVKLDQFPGKVASGTVIRNVRPRR

SEQ ID NO: 55 - protein, 421 aa, Staphylococcus felis
MNKIPKDKGLDNTLKIVKEAYTYVPRRLEAFDTNVFETRALGMKKVLVVSGKKAAELFYDNEKISRKGALPKRIVNTLFGQGAIHTTEGKQHVDRKALFMSLMTTKNLKYLRELTRNYWFAHTQRMEDMDEVNVYEEATLLLTKIGFRWAGLKQTHEEAVQNAKDMDLLIDSFSALGQSPAGYKKAKKARERVEKFLERQIKAVREGKQFAAPNTALYEFAHWKDLNDKPMDLHLCAVDLMNVVRPLVAVNRFVSYAVKALIEFDQERLKLQVTKDQNYAYKFAQEVRRIFPFVPFLPGRLKQNIEFDGYSLKKNTLIILDVFGTTHDPKLFENPYQFNPNRFEDWDGSPFDLIPQGGGDFYTNHRCAGEWMTVIVMEETINYFANKIDFSVPTQDLSVKLDQFPGKVTSGTVIRNVRPRR

SEQ ID NO: 56 - protein, 421 aa, Staphylococcus felis
MNKIPKDKGLDNTLKIVKEAYTYVPRRLEAFDTNIFETRALGMKKVLVVSGKKAAELFYDNEKISRKGALPKRIVNTLFGQGAIHTTEGKQHVDRKALFMSLMTTKNLKYLRELTRNYWFAHTQRMEDMDEVNVYEEATLLLTKIGFRWAGLKQTHEEAVQNAKDMDLLIDSFSALGQSPAGYKKAKKARERVEKFLEHQIKAVREGEQYAAPNTALYEFAHWKDLNDKPMDLHLCAVDLMNVVRPLVAVNRFVSYAVKALIEFDQERLKLQVTKDQNYAYKFAQEVRRIFPFVPFLPGRLKQNIEFDGYSLKKNTLIILDVFGTTHDPKLFENPYQFNPNRFEDWDGSPFDLIPQGGGDFYTNHRCAGEWMTVIVMEETINYFANKIDFSVPTQDLSVKLDQFPGKVTSGTVIRNVRPRR

SEQ ID NO: 57 - protein, 421 aa, Staphylococcus felis
MNKIPKDKGLDNTLKIVKEAYTYVPRRLEAFDTNIFETRALGMKKVLVVSGKKAAELFYDNEKISRKGALPKRIVNTLFGQGAIHTTEGKQHVDRKALFMSLMTTKNLKYLRELTRNYWFAHTQRMEDMDEVNVYEEATLLLTKIGFRWAGLKQTHEEAVQNAKDMDLLIDSFSALGQSPAGYKKAKKARERVEKFLEHQIKAVREGEQFAAPNTALYEFAHWKDLNDKPMDLHLCAVDLMNVVRPLVAVNRFVSYAVKALIEFDQERLKLQVTKDQNYAYKFAQEVRRIFPFVPFLPGRLKQNIEFDGYSLKKNTLIILDVFGTTHDPKLFENPYQFNPNRFEDWDGSPFDLIPQGGGDFYTNHRCAGEWMTVIVMEETINYFANKIDFSVPTQDLSVKLDQFPGKVTSGTVIRNVRPRR

SEQ ID NO: 58 - protein, 421 aa, Staphylococcus felis
MNKIPKDKGLDNTLKIVKEAYTYVPRRLEAFDTNIFETRALGMKKVLVVSGKKAAELFYDNEKISRKGALPKRIVNTLFGQGAIHTTEGKQHVDRKALFMSLMTTKNLKYLRELTRNYWFAHTQRMEDMDEVNVYEEATLLLTKIGFRWAGLKQTHEEAVQNAKDMDLLIDSFSALGQSPAGYKKAKKARERVEKFLEHQIKAVREGEQYAAPNTALYEFAHWKDLNDKPMDLHLCAVDLMNVVRPLVAVNRFVSYAVKALIEFDQERLKLQVTKDQNYAYKFAQEVRRIFPFVPFLPGRLKQNIEFDGYSLKKNTLIILDVFGTTHDPKLFENPYQFNPNRFEDWDGSPFDLIPQGGGDFYTNHRCAGEWMTVIVMEETINYFANKIDFSVPTQDLSVKLDQFPGKVTSGTVIRNVRLRR

SEQ ID NO: 59 - protein, 421 aa, Staphylococcus felis
MNKIPKDKGLDNTLKIVKEAYTYVPRRLEAFDTNVFETRALGMKKVLVVSGKKAAELFYDNEKISRKGALPKRIVNTLFGQGAIHTTEGKQHVDRKALFMSLMTTKNLKYLRELTRNYWFAHTQRMEDMNEVNVYEEATLLLTKIGFRWAGLKQTHEEAVQNAKDMDLLIDSFSALGQSPAGYKKAKKARERVEKFLEHQIKAVREGEQFAAPNTALYEFAHWKDLNDKPMDIHLCAVDLMNVVRPLVAVNRFVSYAVKALIEFDQERLKLQVTKDQNYAYKFAQEVRRIFPFVPFLPGRLKQNIEFDGYSLKKNTLIILDVFGTTHDPKLFENPYQFNPNRFEDWDGSPFDLIPQGGGDFYTNHRCAGEWMTVIVMEETINYFANKIDFSVPTQDLSVKLDQFPGKVTSGTVIRNVRPRR

SEQ ID NO: 60 - protein, 421 aa, Staphylococcus felis
MNKIPKDKGLDNTLKIVKEAYTYVPRRLEAFDTNVFETRALGMKKVLVVSGKKAAELFYDNEKISRKGALPKRIVNTLFGQGAIHTTEGKQHVDRKALFMSLMTTKNLKYLRELTRNYWFAHTQRMEDMDEVNVYEEATLLLTKIGFRWAGLKQTHEEAVQNAKDMDLLIDSFSALGQSPAGYKKAKKARERVEKFLEHQIKAVREGEQYAAPNTALYEFAHWKDLNDKPMDLHLCAVDLMNVVRPLVAVNRFVSYAVKALIEFDQERLKLQVTKDQNYAYKFAQEVRRIFPFVPFLPGRLKQNIEFDGYSLKKNTLIILDVFGTTHDPKLFENPYQFNPNRFEDWDGSPFDLIPQGGGDFYTNHRCAGEWMTVIVMEETINYFANKIDFSVPTQDLSVKLDQFPGKVTSGTVIRNVRPRR

SEQ ID NO: 61 - protein, 421 aa, Staphylococcus felis
MNKIPKDKGLDNTLKIVKEAYTYVPRRLEAFDTNVFETRALGMKKVLVVSGKKAAELFYDNEKISRKGALPKRIVNTLFGQGAIHTTEGKQHVDRKALFMSLMTTKNLKYLRELTRNYWFAHTQRMEDMDEVNVYEEATLLLTKIGFRWAGLKQTHEVAVQNAKDMDLLIDSFSALGQSPAGYKKAKKARERVEKFLEHQIKAVREGEQYAAPNTALYEFAHWKDLNDKPMDLHLCAVDLMNVVRPLVAVNRFVSYAVKALIEFDQERLKLQVTKDQNYAYKFAQEVRRIFPFVPFLPGRLKQNIEFDGYSLKKNTLIILDVFGTTHDSKLFENPYQFNPNRFEDWDGSPFDLIPQGGGDFYTNHRCAGEWMTVIVMEETINYFANKIDFSVPTQDLSVKLDQFPGKVTSGTVIRNVRPRR

SEQ ID NO: 62 - protein, 421 aa, Staphylococcus felis
MNKIPKDKGLDNTLKIVKEAYTYVPRRLEAFDTNVFETRALGMKKVLVVSGKKAAELFYDNEKISRKGALPKRIVNTLFGQGAIHTTEGKQHVDRKALFMSLMTTKNLKYLRELTRNYWFAHTQLMEDMDEVNVYEEATLLLTKIGFRWAGLKQTHEEAVQNAKDMDLLIDSFSALGQSPAGYKKAKKARERVEKFLEHQIKAVREGEQYAAPNTALYEFAHWKDLNDKPMNLHLCAVDLMNVVRPLVAVNRFVSYAVKALIEFDQERLKLQVTKDQNYAYKFAQEVRRIFPFVPFLPGRLKQNIEFDGYSLKKNTLIILDVFGTTHDPKLFENPYQFNPNRFEDWDGSPFDLIPQGGGDFYTNHRCAGEWMTVIVMEETINYFANKIDFSVPTQDLSVKLDQFPGKVTSGTVIRNVRPRR

SEQ ID NO: 63 - protein, 421 aa, Staphylococcus felis
MNKIPKDKGLDNTLKIVKEAYTYVPRRLEAFDTNVFETRALGMKKVLVVSGKKAAELFYDNEKISRKGALPKRIVNTLFGQGAIHTTEGKQHVDRKALFMSLMTTKNLKYLRELTRNYWFAHTQRMEDMDEVNVYEEATLLLTKIGFRWAGLKQTHEEAVQNAKDMDLLIDSFSALGQSPAGYKKAKKARERVEKFLEHQIKAVREGEQYAAPNTALYEFAHWKDLNDKPMNLHLCAVDLMNVVRPLVAVNRFVSYAVKALIEFDQERLKLQVTKDQNYAYKFAQEVRRIFPFVPFLPGRLKQNIEFDGYSLKKNTLIILDVFGTTHDPKLFENPYQFNPNRFEDWDGSPFDLIPQGGGDFYTNHRCAGEWMTVIVMEETINYFANKIDFSVPTQDLSVKLDQFPGKVTSGTVIRNVRPRR

SEQ ID NO: 64 - protein, 424 aa, Fictibacillus enclensis
MSKQLNIPKEEGLDHSLALIQEGYQYITSRRTKFKSDLFETRLLGQKVVCIGGQEGAELFYDNEKFERKGAVPKRIQKSLFGENAIHTLDGEAHEHRKLLFMSLMTEERLELLAELTREEWRLAAAEWAKMEQVVLFDEVQQILCRVACRWAGVPIDAIEVKPLARDFIAMVDAFGAVGPRHQKGRKARKKMEDYFKGLIIEIRHRRIEPIEGTALHKIAWHRDHKEKHLDEHTAAVELLNVLRPIVAITYLVTFGALALFEHNVWKEKLQSGDEESVQMFAQEVRRFYPFAPFLGTKVKNGFEWKGHTFEKGQLVLLDVYGTNHDNRLWEEPYVFNPERFRNWNGGMFDLIPQGGGDEYTGHRCPGEMATIQVMKTSFSFLTNEMIYNVPVGQDLSYSLSRMPTYPESGFIIQGVSIKSQTTV

SEQ ID NO: 65 - protein, 424 aa, Fictibacillus sp. S7
MSKQLNIPKEEGLDHSLALIQEGYQYITSRRTKFKSDLFETRLLGQKVVCIGGQEGAELFYDNEKFERKGAVPKRIQKSLFGENAIHTLDGEAHEHRKLLFMSLMTEERLELLAELTREEWRLAAAEWAKMEQVVLFDEVQQILCRVACRWAGVPIDAIEVKPLARDFIAMVDAFGAVGPRHQKGRKARKKMEDYFKGLIIEIRHRRIEPIEGTALHKIAWHRDHKEKHLDEHTAAVELLNVLRPIVAITYLVTFGALALFEHNVWKEKLQSGDEESVQMFAQEVRRFYPFAPFLGAKVKNGFEWKGHTFEKGQLVLLDVYGTNHDNRLWEEPYVFNPERFRNWNGGMFDLIPQGGGDEYTGHRCPGEMATIQVMKTSFSFLTNEMIYNVPVGQDLSYSLSRMPTYPESGFIIQGVSIKSQTTV

SEQ ID NO: 66 - protein, 420 aa, Lentibacillus salicampi
MKGVAPVAIPRENVLDNTLTLLREGYHYIPGRRRHFKSDIFQTRLLGQKVICIGGKEAAEVFYDEDKFTRIGAIPNRMKESLFGKNGVHVLEGNAHSHRKTMFMSLMTSERLSELTALTEKQWEVAIADWQQKRRVILFPEAEKIMCRIACEWAGVPLWANELSQRTRDLSAMIDAFGAIGPRHWQGRIARNRSERWIRRIIAQVRSGELKPREETALYTFAWHRDLQDRLLDLQTAAVELLNIVRPIVAVGRFVTFGALAINDFPETKEKLRVGDDDYIRMFVQEVRRYYPFGPFLGAKVRSNFKWTGHQFKKGTLVLLDIYGTNHDPDLWDKPDEFRPERFDEWEGSPFDFIPQGGGYDMGHRCPGEWITIDIMKVSLAFLANRIDYNVPKQNLDYSMVRMPSIPKSRFIMRDVAISS

SEQ ID NO: 67 - protein, 426 aa, Staphylococcus vitulinus
MGKQIPKDRGLDSTLKVLKEGYKYVPNRLEKFDTNIFELRALGGRRTVVISGKEAAELFYNNELTERQGTLPKRVVNTLFGKGAIHTTGGKKHIDRKALFMSLMTEENLDYLRELTRSTWFMNTERMERMDEVNVYKESIILLTKVGFRWAGIIASPEEIESCAQDMDTMIDSFKNIGTVFKGYREAKQARARVETFLENQIIAVREGKLTPPQGTALHEFSHWEDFEGNLMDSRLCAIDLMNVVRPLVAINRFISFGVKALHDYPGEAEKVFNNEDDYAYKFVQEIRRFYPFVPFLPGKAAVDIEFEGYTIEKDTFLVLDIYGTLHREGLWENPERFYPNRFSDWDGSPFDLIPQGGGDYHTNHRCAGEWMTLIIMEESMKYFARNISYDVKKDQDLSVNLNKLPGRVVSGTIIENVNALVNRNV

SEQ ID NO: 68 - protein, 429 aa, Staphylococcus sciuri
MGKQIPKDRGLDSTFKVLKEGYKYVPNRLEKFDTNIFELRALGGRRTVVISGKEAAELFYNNDLIERQGTLPKRVVNTLFGKGAIHTTGGKKHIDRKALFMSLMTEENLEYLRELTRSTWFMNTERMERMDEVNVYKESIILLTKVGFRWAGVIASPEEIESCAQDMDTMIDSFKNIGTAFKGYREAKKARDRVETFLENQIIAVREGKLTPPQGTALHEFSHWEDFEGNLMDSRLCAIDLMNVVRPLVAINRFISFGVKALHDYPGEAEKVFNNENDYAYKFVQEVRRFYPFVPYLPGKAAVDIEFDGYTIEKDTFLVLDIYGTLHRDGLWENPERFYPNRFSDWDGSPFDLIPQGGGDYYTNHRCAGEWMTIIIMEESMKYFARNISYDVKKDQDLSVNLNKLPGRVVSGTIIENVNALVNRNVESV

SEQ ID NO: 69 - protein, 429 aa, Staphylococcus sciuri
MGKQIPKDRGLDSTLKVLKEGYKYVPNRLEKFDTNIFELRALGGRRTVVFSGKEAAEIFYNNELIERQGTLPKRVVNTLFGKGAIHTTGGKKHIDRKALFMSLMTEENLEYLRELTRSTWFMNTERMERMDEVNVYKESIILLTKVGFRWAGIIASPEEIESCAKDMDTMIDSFKNIGTAFKGYREAKKARDRVETFLENQIIAVREGKLTPPQGTALHEFSHWEDFEGNLMDSRLCAIDLMNVVRPLVAINRFVSFGVKALHDYPGEAEKVFNNENDYAYKFVQEVRRFYPFVPFLPGKAAVDIEFDGYKIEKDTFLVLDIYGTLHREGLWENPERFYPNRFSDWDGSPFDLIPQGGGDYYTNHRCAGEWMTIIIMEESMKYFARNISYDMKKDQDLSVNLNKLPGRVVSGTIIENVNALVNRNVESV

SEQ ID NO: 70 - protein, 429 aa, Staphylococcus sciuri
MGKQIPKDRGLDSTFKVLKEGYKYVPNRLEKFDTNIFELRALGGRRTVVISGKEAAELFYNNDLIERQGTLPKRVVNTLFGKGAIHTTGGKKHIDRKALFMSLMTEENLEYLRELTRSTWFMNTERMERMDEVNVYKESIILLTKVGFRWAGVIASPEEIESCAQDMDTMIDSFKNIGTAFKGYREAKKARDRVETFLENQIIAVREGKLTPPQGTALHEFSHWEDFEGNLMDSRLCAIDLMNVVRPLVAINRFISFGVKALHDYPGEAEKVFNNENDYAYKFVQEVRRFYPFVPYLPGKAAVNIEFDGYTIEKDTFLVLDIYGTLHRDGLWENPERFYPNRFSDWDGSPFDLIPQGGGDYYTNHRCAGEWMTIIIMEESMKYFARNISYDMKKDQDLSVNLNKLPGRVVSGTIIENVNALVNRNVESV

SEQ ID NO: 71 - protein, 429 aa, Staphylococcus sp.
MGKQIPKDRGLDSTLKVLKEGYKYVPNRLEKFDTNIFELRALGGRRTVVFSGKEAAEIFYNNELIERQGTLPKRVVNTLFGKGAIHTTGGKKHIDRKALFMSLMTEENLEYLRELTRSTWFMNTERMERMDEVNVYKESIILLTKVGFRWAGIIASPEEIESCAKDMDTMIDSFKNIGTAFKGYREAKKARDRVETFLENQIIAVREGKLTPPQGTALHEFSHWEDFEGNLMDSRLCAIDLMNVVRPLVAINRFVSFGVKALHDYPGEAEKVFNNENDYAYKFVQEVRRFYPFVPFLPGKAAVDIEFDGYTIEKDTFLVLDIYGTLHREGLWENPERFYPNRFSDWDGSPFDLIPQGGGDYYTNHRCAGEWMTIIIMEESMKYFARNISYDMKKDQDLSVNLNKLPGRVVSGTIIENVNALVNRNVESV

SEQ ID NO: 72 - protein, 429 aa, Staphylococcus sciuri
MGKQIPKDRGLDSTFKVLKEGYKYVPNRLEKFDTNIFELRALGGRRTVVISGKEAAELFYNNDLIERQGTLPKRVVNTLFGKGAIHTTGGKKHIDRKALFMSLMTEENLEYLRELTRSTWFMNTERMERMDEVNVYKESIILLTKVGFRWAGVIASPEEIESCAQDMDTMIDSFKNIGTAFKGYREAKKARDRVETFLENQIIAVREGKLTPPQGTALHEFSHWEDFEGNLMDSRLCAIDLMNVVRPLVAINRFISFGVKALHDYPGEAEKVFNNENDYAYKFVQEVRRFYPFVPYLPGKAAVDIEFDGYTIEKDTFLVLDIYGTLHRDGLWDNPERFYPNRFSDWDGSPFDLIPQGGGDYYTNHRCAGEWMTIIIMEESMKYFARNISYDMKKDQDLSVNLNKLPGRVVSGTIIENVNALVNRNVESV

SEQ ID NO: 73 - protein, 429 aa, Staphylococcus sciuri
MGKQIPKDRGLDSTFKVLKEGYKYVPNRLEKFDTNIFELRALGGRRTVVISGKEAAELFYNNDLIERQGTLPKRVVNTLFGKGAIHTTGGKKHIDRKALFMSLMTEENLEYLRELTRSTWFMNTERMERMDEVNVYKESIILLTKVGFRWAGVIASPEEIESCARDMDTMIDSFKNIGTAFKGYREAKKARDRVETFLENQIIAVREGKLTPPQGTALHEFSHWEDFEGNLMDSRLCAIDLMNVVRPLVAINRFISFGVKALHDYPGEAEKVFNNENDYAYKFVQEVRRFYPFVPYLPGKAAVDIEFDGYTIEKDTFLVLDIYGTLHRDGLWENPERFYPNRFSDWDGSPFDLIPQGGGDYYTNHRCAGEWMTIIIMEESMKYFARNISYDMKKDQDLSVNLNKLPGRVVSGTIIENVNALVNRNVESV

SEQ ID NO: 74 - protein, 429 aa, Staphylococcus sp.
MGKQIPKDRGLDSTFKVLKEGYKYVPNRLEKFDTNIFELRALGGRRTVVISGKEAAELFYNNDLIERQGTLPKRVVNTLFGKGAIHTTGGKKHIDRKALFMSLMTEENLEYLRELTRSTWFMNTERMERMDEVNVYKESIILLTKVGFRWAGVIASPEEIESCAQDMDTMIDSFKNIGTAFKGYREAKKARDRVETFLENQIIAVREGKLTPPQGTALHEFSHWEDFEGNLMDSRLCAIDLMNVVRPLVAINRFISFGVKALHDYPGEAEKVFNNENDYAYKFVQEVRRFYPFVPYLPGKAAVDIEFDGYTIEKDTFLVLDIYGTLHRDGLWENPERFYPNRFSDWDGSPFDLIPQGGGDYYTNHRCAGEWMTIIIMEESMKYFARNISYDMKKDQDLSVNLNKLPGRVVSGTIIENVNALVNRNVESV

SEQ ID NO: 75 - protein, 429 aa, Staphylococcus vitulinus
MGKQIPKDRGLDSTLKVLKEGYKYVPNRLEKFDTNIFELRALGGRRTVVISGKEAAELFYNNELTERQGTLPKRVVNTLFGKGAIHTTGGKKHIDRKALFMSLMTEENLDYLRELTRSTWFMNTERMERMDEVNVYKESIILLTKVGFRWAGIIASPEEIESCAHDMDTMIDSFKNIGTVFKGYREAKQARARVETFLENQIIAVREGKLTPPQGTALHEFSHWEDFEGNLMDSRLCAIDLMNVVRPLVAINRFISFGVKALHDYPGEAEKVFNNEDDYAYKFVQEIRRFYPFVPFLPGKAAVDIEFEGYTIEKDTFLVLDIYGTLHREGLWENPERFYPNRFSDWDGSPFDLIPQGGGDYHTNHRCAGEWMTLIIMEESMKYFARNISYDVKKDQDLSVNLNKLPGRVVSGTIIENVNALVNRNVESV

SEQ ID NO: 76 - protein, 429 aa, Staphylococcus vitulinus
MGKQIPKDRGLDSTLKVLKEGYKYVPNRLEKFDTNIFELRALGGRRTVVISGKEAAELFYNNELTERQGTLPKRVVNTLFGKGAIHTTGGKKHIDRKALFMSLMTEENLDYLRELTRSTWFMNTERMERMDEVNVYKESIILLTKVGFRWAGIIASPEEIESCAQDMDTMIDSFKNIGTVFKGYREAKQARARVETFLENQIIAVREGKLTPPQGTALHEFSHWEDFEGNLMDSRLCAIDLMNVVRPLVAINRFISFGVKALHDYPGEAEKVFNNEDDYAYKFVQEIRRFYPFVPFLPGKAAVDIEFEGYTIEKDTFLVLDIYGTLHREGLWENPERFYPNRFSDWDGSPFDLIPQGGGDYHTNHRCAGEWMTLIIMEESMKYFARNISYDVKKDQDLSVNLNKLPGRVVSGTIIENVNALVNRNVESV

SEQ ID NO: 77 - protein, 429 aa, Staphylococcus sp.
MGKQIPKDRGLDSTLKVLKEGYIYVPNRLEKFDTNIFELRALGGRRTVVISGKEAAELFYNNELTERQGTLPKRVVNTLFGKGAIHTTGGKKHIDRKALFMSLMTEENLDYLRELTRSTWFMNTERMERMDEVNVYKESIILLTKVGFRWAGIIASPEEIESCAHDMDTMIDSFKNIGTVFKGYREAKQARARVETFLENQIIAVREGKLTPPQGTALHEFSHWEDFEGNLMDSRLCAIDLMNVVRPLVAINRFISFGVKALHDYPGEAEKVFNNEDDYAYKFVQEIRRFYPFVPFLPGKAAVDIEFEGYTIEKDTFLVLDIYGTLHREGLWENPERFYPNRFSDWDGSPFDLIPQGGGDYHTNHRCAGEWMTLIIMEESMKYFARNISYDVKKDQDLSVNLNKLPGRVVSGTIIENVNALVNRNVESV

SEQ ID NO: 78 - protein, 429 aa, Staphylococcus sciuri
MGKQIPKDRGLDSTFKVLKEGYKYVPNRLEKFDTNIFELRALGGRRTVVISGKEATELFYNNDLIERQGTLPKRVVNTLFGKGAIHTTGGKKHIDRKALFMSLMTEENLEYLRELTRSTWFMNTERMERMDEVNVYKESIILLTKVGFRWAGVIASPEEIESCAQDMDTMIDSFKNIGTAFKGYREAKKARDRVETFLENQIIAVREGKLTPPQGTALHEFSHWEDFEGNLMDSRLCAIDLMNVVRPLVAINRFISFGVKALHDYPGEAEKVFNNENDYAYKFVQEVRRFYPFVPYLPGKAAVDIEFDGYTIEKDTFLVLDIYGTLHRDGLWENPERFYPNRFSDWDGSPFDLIPQGGGDYYTNHRCAGEWMTIIIMEESMKYFARNISYDMKKDQDLSVNLNKLPGRVVSGTIIENVNALVNRNVESV

SEQ ID NO: 79 - protein, 429 aa, Staphylococcus stepanovicii
MGKQIPKERGLDSTFKVLKEGYKYVPNRLEKHNSNIFELRALGGRRTVVFSGKEAAEVFYNNDLIERQGTLPKRVVNTLFGKGAIHTTGGKKHIDRKALFMSLMTEENLDYLRELTRSFWFTNIERMERMDEVNVYKESIILLTKVGFRWAGVIASPEEIERCAEDMDTMIDSFKNIGTAFKGYKEAKQARDRVETFLENQIIAVREGKLTPPQGTALYEFSHWEDFEGNPMDSRLCAIDLMNVVRPLVAINRFVSFGVKALHDYPGEAEKVFNNENDYAYKFVQEVRRFYPFVPFLPGRAAVDIEFEDYTIEKGTFLVLDIYGTLHKEGLWENPERFYPNRFSDWDGSPFDLIPQGGGDYNTNHRCAGEWMTIIIMEESMKFFAKDISYDVKKDQDQSVNLNKLPGRVASGMIIENVNALVNRNVESV

SEQ ID NO: 80 - protein, 429 aa, Staphylococcus sp.
MGKQIPKDRGLDSTFKVLKEGYNYVPSRLEKFDSNIFELRALGGRRTVVISGKEAAELFYNNELIERQGTLPKRVVNTLFGKGAIHTTGGKKHIDRKALFMSLMTEENLDYLRELTRSTWFMNTERMERMDEVNVYKESIVLLTKVGFRWAGIIASPEEIESCAKDMDTMIDSFKNIGTAFKGYKEAKQARARVETFLENQIIAVREGKLTPPQGTALHEFSHWEDFEGNLMDSRLCAIDLMNVVRPLVAINRFVSFGVKALHDYPGEAEKVFNNEDDYAYKFVQEVRRFYPFVPFLPGKAAVDIEFDGYTIEKDTFLVMDIYGTLHKEGLWENPERFYPNRFSDWDGSPFDLIPQGGGDYNTNHRCAGEWMTIIIMEESMKYFARNISYDMKQDQDLSVKLNKLPGRVVSGTIIENVNALVNRNVESV

SEQ ID NO: 81 - protein, 429 aa, Staphylococcus fleurettii
MGKQIPKDRGLDSTFKVLKEGYNYVPSRLEKFDSNIFELRALGGRRTVVISGKEAAELFYNNELIERQGTLPKRVVNTLFGKGAIHTTGGKKHIDRKALFMSLMTEENLDYLRELTRSTWFMNTERMERMDEVNVYKESIVLLTKVGFRWAGIIASPEEIESCAKDMDTMIDSFKNIGTAFKGYKEAKQARARVETFLENQIIAVREGKLTPPQGTALHEFSHWEDFEGNLMDSRLCAIDLMNVVRPLVAINRFVSFGVKALHDYPGEAEKVFNNEDDYAYKFVQEVRRFYPFVPFLPGKAAVDIEFDGYTIEKDTFLIMDIYGTLHKEGLWENPERFYPNRFSDWDGSPFDLIPQGGGDYNTNHRCAGEWMTIIIMEESMKYFARNISYDMKQDQDLSVKLNKLPGRVVSGTIIENVNALVNRNVESV

SEQ ID NO: 82 - protein, 425 aa, Abyssicoccus albus
MGKSIPRDRGMDNTVKALKKGYTYIPELRERLNSNDIVATRALGGKKTVIISGKDAAELFYDNDKIERQGTLPPRVVKTLFGKGAIHTTTGKTHIDRKALFMSLMTEENLSYLRELTRLEWYYNTTRMSHMDEVNVYKEAIYVLTKVGIKWAGLHASEDEINQYAEDMDTMIDSFKAIGTMFKGYKEARKARDRVETWLEAQIQGVRDGEITPPEGTALYEFAHWKDYEGNPMDARLAAIDLMNVIRPLVAINRFVAFGTLAMYEFPKERTKVAQNEEDYVYKFIQEVRRFYPFVPYLPGKAGKDIEFRGYEIKKDTFMVLDVYGTLHREDLWENADQFIPERFEDWDGSPFDLIPQGGGDYYTNHRCAGEWMTVIVMEESMKYFAQAIKYEVPAQDLTVDLNKLPGRVKSGFIIKNVERDINRF

SEQ ID NO: 83 - protein, 425 aa, Auricoccus indicus
MGKSIPRDRGMDNTVKALKKGYTYIPELRERLNSNDIVATRALGGKKTVIISGKDAAELFYDNDKIERQGTLPPRVVKTLFGKGAIHTTSGKTHIDRKALFMSLMTEENLSYLRELTRLEWYYNTTRMSHMDEVNVYKEAIYVLTKVGIKWAGLHASEDEINQYAEDMDTMIDSFKAIGTMFKGYKEARKARDRVETWLEAQIQGVRDGEITPPEGTALYEFAHWKDYEGNPMDARLAAIDLMNVIRPLVAINRFVAFGTLAMYEFPKERTKVAQNEEDYVYKFIQEVRRFYPFVPYLPGKAGKDIEFRGYEIKKDTFMVLDVYGTLHREDLWENADQFIPERFEDWDGSPFDLIPQGGGDYYTNHRCAGEWMTVIVMEESMKYFAQAIKYEVPAQDLTVDLNKLPGRVKSGFIIKNVERDINRF

SEQ ID NO: 84 - protein, 423 aa, Aliicoccus persicus
MSTIKKDKGLDNTLKAIKQGYKYTENQRKRLDSNNIFETRGLGGKRIIFISGKEAAETFYDNDKIEREGMLPKRVVSTLFGKGAIHTTTGKVHIDRKALFMSLMTEGNLQYLRELTRNQWRSNTINMERQDTVNVYREAIVQLTHIGTRWAGVQAPAEEIDRIATDMDLMIDSFKGLGLSFKGFRESKAARRRVEDWLEDQIHKTRKGVIHPPEGTALYEFAHWEDFEGNPMDSRLCAIDLMNTFRPLIAINRFVTFGVLALHQYPETREKIKNDPDYAYMFSQEVRRYYPFVPFLPAKAKVDFEFEGHKIDKDVMILLDVYGTMHRDDVFDNPNEFYPERFKDWDGSPFDLIPQGGGDYHTNHRCAGEWMTIIIMEETMKYFAERITYDVPEQDLTVDLEKLPGSVASGMNITNVRENVNRK

SEQ ID NO: 85 - protein, 423 aa, Staphylococcus lutrae
MNKKLPKDTGLDNTLKIINEAYTYVPKRLEKFGTKAFETRALGMKPITVMSGKAAAELFYDNDKVSRTGTLPKRVVHTLFGKGAIHTTEGKVHVNRKALFMSLMTEKNLKYLRELTRHYWFMHTERMQNMDEVSVYKEAGIILTKVGFRWAGLKQTDEEAVKNAEDMNKMIDSFSRLGQSVKGYRQAKKARARVEQFLRDQIKAVRENKQYAEPGTALYEFAHWKDLKGQPMDLHLCAVDLMNIVRPLVAVNRFISYAVKALIEFDQERQKLQVTHDPNYAYKFAQEVRRIFPFVPFLPGKLKKTIDFDGFKIKKGTMVALDIFGTTHDPELFKNPYQFNPNRFEKWDGSPFDLIPQGGGDFYTNHRCAGEWMTIIVMEETVQYFANKIDFEVPAQDLSVKLDRFPGKVTSGTLIKNVRPRRS

SEQ ID NO: 86 - protein, 425 aa, Staphylococcus aureus
MAKQLPKDPGLDNTFKVLKEAYTYVPCRLEKFNSKAFQTTGMGMKPIAVISGKEAAELFYNNDVMQREKTLPKRVVNTLFGKGAIHTTKGKVHVDRKALFMSLMTEENLKYLRELTRNHWFMHTEHMQNQKEVNIYKESIYVLTKIGFRWAGIHQTTEEAEQNAKDMDIMIDSFKGLGQTIGGGYRKAKKARARVEQFLEKQIIAVRKGKISAEPGTALYEFAHWEDYKGNPMDARLCAIDLMNVVRPLAAVNRFVSYAVKAMIEYDQERIKLQVSDDPNYAYKFAQEVRRIFPFVPFLPGKLKKNIEFDGYRIKKGTFTLLDVFGTTHDPKLFENPYQFNPDRFENWDGSPFDLIPQGGGDFYTNHRCAGEWMTIIVMEETIKYFANKIDFDAPSQDLSVKLDQFPGKVTSGTIIQNVRPRVQR

SEQ ID NO: 87 - protein, 425 aa, Staphylococcus agnetis
MAKQLPKDPGLDNTFKVLKEAYTYVPCRLEKFNSKAFQTTGMGMKPIAVISGKDAAELFYNNDVMQREKTLPKRVVNTLFGKGAIHTTKGKVHVDRKALFMSLMTEENLKYLRELTRNHWFMHTEHMQNQKEVNIYKESIYVLTKIGFRWAGIHQTAEEAEQNAKDMDIMIDSFKGLGQTIGGGYRKAKKARARVEQFLEKQIIAVRKGKINAEPGTALYEFAHWEDYKGNPMDARLCAIDLMNVVRPLAAVNRFVSYAVKAMIEYDQERIKLQVSDDPNYAYKFAQEVRRIFPFVPFLPGKLKKNIEFDGYRIKKGTFTLLDVFGTTHDPELFENPYQFNPDRFENWDGSPFDLIPQGGGDFYTNHRCAGEWMTIIVMEETIKYFANKIDFEAPSQDLSVKLDQFPGKVTSGTIIQNVRPRVQR

SEQ ID NO: 88 - protein, 425 aa, Staphylococcus agnetis
MAKQLPKDPGLDNTFKVLKEAYTYVPCRLEKFNSKAFQTTGMGMKPIAVISGKEAAELFYNNDVMQREKTLPKRVVNTLFGKGAIHTTKGKVHVDRKALFMSLMTEENLKYLRELTRNHWFMHTEHMQNQKEVNIYKESIYVLTKIGFRWAGIHQTAEEAEQNAKDMDIMIDSFKGLGQTIGGGYRKAKKARARVEQFLEKQIIAVRKGKINAEPGTALYEFAHWEDYKGNPMDARLCAIDLMNVVRPLAAVNRFVSYAVKAMIEYDQERIKLQVSDDPNYAYKFAQEVRRIFPFVPFLPGKLKKNIEFDGYRIKKGTFTLLDVFGTTHDPELFENPYQFNPDRFENWDGSPFDLIPQGGGDFYTNHRCAGEWMTIIVMEETIKYFANKIDFEAPSQDLSVKLDQFPGKVTSGTIIQNVRPRVQR

SEQ ID NO: 89 - protein, 425 aa, Staphylococcus agnetis
MAKQLPKDPGLDNTFKVLKEAYTYVPCRLEKFNSKAFQTTGMGMKPIAVISGKEAAELFYNNDVMQREKTLPKRVVNTLFGKGAIHTTKGKVHVDRKALFMSLMTEENLKYLRELTRNHWFMHTEHMQNQREVNIYKESIYVLTKIGFRWAGIHQTAEEAEQNAKDMDIMIDSFKGLGQTIGGGYRKAKKARARVEQFLEKQIIAVRKGKINAEPGTALYEFAHWEDYKGNPMDARLCAIDLMNVVRPLAAVNRFVSYAVKAMIEYDQERIKLQVSDDPNYAYKFAQEVRRIFPFVPFLPGKLKKNIEFDGYRIKKGTFTLLDVFGTTHDPELFENPYQFNPDRFENWDGSPFDLIPQGGGDFYTNHRCAGEWMTIIVMEETIKYFANKIDFEAPSQDLSVKLDQFPGKVTSGTIIQNVRPRVQR

SEQ ID NO: 90 - protein, 425 aa, Staphylococcus agnetis
MAKQLPKDPGLDNTFKVLKEAYTYVPCRLEKFNSKAFQTTGMGMKPIAVISGKEAAELFYNNDVMQREKTLPKRVVNTLFGKGAIHTTKGKVHVDRKALFMSLMTEENLKYLRELTRNHWFMHTEHMQNQKEVNIYKESIYVLTKIGFRWAGIHQTTEEAEQNAKDMDIMIDSFKGLGQTIGGGYRKAKKARARVEQFLEKQIIAVRKGKINAEPGTALYEFAHWEDYKGNPMDARLCAIDLMNVVRPLAAVNRFVSYAVKAMIEYDQERIKLQVSDDPNYAYKFAQEVRRIFPFVPFLPGKLKKNIEFDGYRIKKGTFTLLDVFGTTHDPELFENPYQFNPDRFENWDGSPFDLIPQGGGDFYTNHRCAGEWMTIIVMEETIKYFANKIDFEAPSQDLSVKLDQFPGKVTSGTIIQNVRPRVQR

SEQ ID NO: 91 - protein, 425 aa, Staphylococcus agnetis
MAKQLPKDPGLDNTFKVLKEAYTYVPCRLEKFNSKAFQTTGMGMKPIAVISGKEAAELFYNNDVMQREKTLPKRVVNTLFGKGAIHTTKGKVHVDRKALFMSLMTEENLKYLRELTRNHWFMHTEHMQNQKEVNIYKESVYVLTKIGFRWAGIHQTAEEAEQNAKDMDIMIDSFKGLGQTIGGGYRKAKKARARVEQFLENQIIAVRKGKINAEPGTALYEFAHWEDYKGNPMDARLCAIDLMNVVRPLAAVNRFVSYAVKAMIEYDQERIKLQVSDDPNYAYKFAQEVRRIFPFVPFLPGKLKKNIEFDGYRIKKGTFTLLDVFGTTHDPELFENPYQFNPDRFENWDGSPFDLIPQGGGDFYTNHRCAGEWMTIIVMEETIKYFANKIDFEAPSQDLSVKLDQFPGKVTSGTIIQNVRPRVQR

SEQ ID NO: 92 - protein, 423 aa, Salinicoccus sediminis
MGTIKRDKGLDNTLKVMKQGYLYITNQRKRLGSENIFETRALGGKKIIVISGKDAAELFYDNDKIERSGTLPKRVVNTLFGKGAIHTTTGKKHIDRKALFMSLMTEGNLKHLRELTRNHWYMNTQKMEQMDQINIYRESIVQLTKIGTKWAGVQAPEEKIEEIATDMDVMIDSFKGLGGKFKGYKASVDARKRVEDWLEDQIIQTRKGKIFPPEGTALYEFAHWEDYKGNPMDSRLCAIDLMNTFRPLIAINRFVSFGLLAMHEHPITREKIKSDDDYAYMFSQEVRRFYPFVPFLPAKAKTDIQFEGHPIEKDTMIALDIYGTMHREDVFENPDDFYPERFKDWDGSPFDLIPQGGGDYYTNHRCAGEWMTIIIMEETMKYFAERVTYDVPEQDLTVDLNSIPGYVKSGFIIENLREVVDRK

SEQ ID NO: 93 - protein, 423 aa, Salinicoccus carnicancri
MGTISRDKGLDNTLKVMKQGYLYMTNQRKRLGSENIFETRALGGKKIIVISGKDAAELFYDNDKIERSGTLPKRVVNTLFGKGAIHTTTGKKHIDRKALFMSLMTEGNLKHLRELTRNHWYMNTQKMEQMEQINIYRESIVQLTKIGTKWAGVQAPEEKIEEIATDMDVMIDSFKGLGGKFKGYKASVDARKRVEDWLEDQIIQTRKGKIFPPEGTALYEFAHWEDYKGNPMDSRLCAIDLMNTFRPLIAINRFVSFGLLAMHEHPITREKIKSDDDYAYMFSQEVRRFYPFVPFLPAKAKTDIQFEGHPIEKDTMIVLDIYGTMHREDVFENPDDFYPERFRDWDGSPFDLIPQGGGDYYTNHRCAGEWMTIIIMEETMKYFAGRVTYDVPEQDLTVDLNSIPGYVKSGFIIENLREVVDRK

SEQ ID NO: 94 - protein, 425 aa, Staphylococcus agnetis
MAKQLPKDPGLDNTFKVLKEAYTYVPCRLEKFNSKAFQTTGMGMKPIAVISGKEAAELFYNNDVMQREKTLPKRVVNTLFGKGAIHTTKGKVHVDRKALFMSLMTEENLKYLRELTRNHWFMHTEHMQNQKEVNIYKESIYVLTKIGFRWAGIHQTTEEAEQNAKDMDIMIDSFKGLGQTIGGGYRKAKKARARVEQFLERQIIAVRKGKINAEPSTALYEFAHWEDYKGNPMDARLCAIDLMNVVRPLAAVNRFVSYAVKAMIEYDQERIKLQVSDDPNYAYKFAQEVRRIFPFVPFLPGKLKKNIEFDGYRIKKGTFTLLDVFGTTHDPELFENPYQFNPDRFENWDGSPFDLIPQGGGDFYTNHRCAGEWMTIIVMEETIKYFANKIDFEAPSQDLSVKLDQFPGKVTSGTIIKNVRPRVQR

SEQ ID NO: 95 - protein, 425 aa, Staphylococcus agnetis
MAKQLPKDPGLDNTFKVLKEAYTYVPCRLEKFNSKAFQTTGMGMKPIAVINGKEAAELFYNNDVMQREKTLPKRVVNTLFGKGAIHTTKGKVHVDRKALFMSLMTEENLKYLRELTRNHWFMHTEHMQNQKEVNIYKESIYVLTKIGFRWAGIHQTAEEAEQNAQDMDIMIDSFKGLGQTIGGGYRKAKKARARVEQFLEKQIIAVRKGKINAEPGTALYEFAHWEDYKGNPMDARLCAIDLMNVVRPLAAVNRFVSYAVKAMIEYDQERIKLQVSDDPNYAYKFAQEVRRIFPFVPFLPGKLKKNIEFDGYRIKKGTFTLLDVFGTTHDPELFENPYQFNPDRFENWDGSPFDLIPQGGGDFYTNHRCAGEWMTIIVMEETIKYFANKIDFEAPSQDLSVKLDQFPGKVTSGTIIQNVRPRVQR

SEQ ID NO: 96 - protein, 425 aa, Staphylococcus agnetis
MAKQLPKDPGLDNTFKVLKEAYTYVPCRLEKFNSKAFQTTGMGMKPIAVINGKEAAELFYNNDVMQREKTLPKRVVNTLFGKGAIHTTKGKVHVDRKALFMSLMTEENLKYLRELTRNHWFMHTEHMQNQKEVNIYKESIYVLTKIGFRWAGIHQTAEEAEQNAQDMDIMIDSFKGLGQTIGGGYRKAKKARARVEQFLEKQIIAVRKGKINAEPGTALYEFAHWEDYKGNPMDARLCAIDLMNVVRPLAAVNRFVSYAVKAMIEYDQERIKLQVSDDPNYAYKFAQEVRRIFPFVPFLPGKLKKNIEFDGYRIKKGTFTLLDVFGTTHDPELFENPYQFNPDRFENWDGSPFDLIPQGGGDFYTNHRCAGEWMTIIVMEETIKYFANKIDFEAPSQDLFVKLDQFPGKVTSGTIIQNVRPRVQR

SEQ ID NO: 97 - protein, 425 aa, Staphylococcus agnetis
MAKQLPKDPGLDNTFKVLKEAYTYVPCRLEKFNSKAFQTTGMGMKPIAVISGKEAAELFYNNDVMQREKTLPKRVVNTLFGKGAIHTTKGKVHVDRKALFMSLMTEENLKYLRELTRNHWFMHTEHMQNQREVNIYKESIYVLTKIGFRWAGIHQTAEEAEQNAKDMDIMIASFKGLGQTIGGGYRKAKKARARVEQFLEKQIIAVRKGKINAEPGTALYEFAHWEDYKGNPMDARLCAIDLMNVVRPLAAVNRFVSYAVKAMIEYDQERIKLQVSDDPNYAYKFAQEVRRIFPFVPFLPGKLKKNIEFDGYRIKKGTFTLLDVFGTTHDPELFENPYQFNPDRFENWDGSPFDLIPQGGGDFYTNHRCAGEWMTIIVMEETIKYFANKIDFEAPSQDLSVKLDQFPGKVTSGTIIQNVRPRVQR

SEQ ID NO: 98 - protein, 424 aa, Staphylococcus sp.
MAKKLPKDSGLDNTFKVLKEAYTYVPCRLEKFNTKAFQTKAMGMKPIVVISGKEAAELFYNNDVMQREKTLPKRVVNTLFGKGAIHTTKGKVHVDRKALFMSLMTEENLKYLRELARNHWFVHTEHMQTQDEVNIYKESIYVLTKIGFRWAGIRQTAEEAERNAKDMDVMIDSFKGLGQTLGSGYRKAKKARARVEQFLEDQIVAVRNGEIKAEPGTALYEFAHWEDYKGNQMDARLCAVDLMNVVRPLAAVNRFVSYAVKAMIEYDQERLKLQVTDDPNYAYKFAQEVRRIFPFVPFLPGKFKKTVEFDGYRIKKGTFTLLDVFGTTHDPDLFENPYQFNPDRFDNWDGSPFDLIPQGGGDFYTNHRCAGEWMTIIVMEETIKYFANKIDFDAPAQDLSVKLDQFPGKVTSGTLIKNVRPRLQ

SEQ ID NO: 99 - protein, 424 aa, Staphylococcus hyicus
MAKKLPKDSGLDNTFKVLKEAYTYVPCRLEKFNTKAFQTKAMGMKPIVVISGKEAAELFYNNDVMQREKTLPKRVVNTLFGKGAIHTTKGKVHVDRKALFMSLMTEENLKYLRELARNHWFVHTEHMQTQDEVNIYKESIYVLTKIGFRWAGIRQTAEEAERNAKDMDVMIDSFKGLGQTLGSGYRKAKKARARVEQFLEDQIVAVRNGEIKAEPGTALYEFAHWEDYKGNQMDARLCAVDLMNVVRPLAAVNRFVSYAVKAMIEYDQECLKLQVTDDPNYAYKFAQEVRRIFPFVPFLPGKFKKTVEFDGYRIKKGTFTLLDVFGTTHDPDLFENPYQFNPDRFDNWDGSPFDLIPQGGGDFYTNHRCAGEWMTIIVMEETIKYFANKIDFDAPAQDLSVKLDQFPGKVTSGTLIKNVRPRLQ

SEQ ID NO: 100 - protein, 424 aa, Salinicoccus qingdaonensis
MATIKRDKGLDNSVKVMKQGYLYTTNQRERLGVTDGVFETRALGGKRIIVLSGKDGAELFYDNDKIERSGTLPKRVVNTLFGKGAIHTTTGKVHIDRKALFMSLMTEGNLKYLRELTRNLWFANTQRMESMDQVNVYRESIIVLTKVGTRWAGVQAPEKEIENIATDMDIMIDSFKAIGSAFKGYREAKAARRRVEDWLEDQIIQTRKGKIHPPKGTALYEFAHWKDYKGEPMDSRLCGIDLMNTFRPLIAINRFVAFGALAMYENPVAREKIKQDDDYAYMFAQEVRRFYPFVPYLPAKAKVDFQYKGYEIEKDTMLALDIYGTMHDPNVWEDPNEFYPERFKDWDGSPFDLIPQGGGDYHTNHRCAGEWMTVIIMEETMKYFASRINYDVPEQDLTVDLNSLPGYIKSGFVIENVQENVDRT

SEQ ID NO: 101 - protein, 424 aa, Salinicoccus alkaliphilus
MATIKKDKGIDNTAKIAKQGYLYTTNQRERLGVKDGVFETRGLGGKRIIILSGKDGAELFYDNDKVERSGTLPKRVVNTLFGKGAIHTTTGKVHVDRKALFMSLMTEGNLQYLRELTRNHWLMNTQRMENMDQVNVYRESIILLTKVGTRWAGVQAPEKEIENIATDMDIMIDSFKAIGSAFKGYRASKAARRRVEDWLEDQIIQTRKGKIHPPKGTALYEFAHWKDYKGEPMDSRLCGIDLMNTFRPLIAINRFIAFGALAMHENPVAREKIKQDDDYAYMFAQEVRRFYPFVPYLPGKVKEDFQYKGYDFEKDTMLAIDIYGTMHDPDVWENPNEFYPERFKDWDGSPFDLIPQGGGDYYTNHRCAGEWMTIIIMQETMKYFASRITYDVPEQDLTVDLNSLPGYVKSGFIIENVREVVDRD

SEQ ID NO: 102 - protein, 425 aa, Staphylococcus chromogenes
MAKKLPKDSGLDNTFKILNEAYTYVPCRLEKFGTKAFETAAIGMKPMTVISGKEAAELFYDNELMQREKTLPKRVVNTLFGKGAIHTTKGKVHVDRKALFMSLMTEENLKYLRDLTRNYWFMHTEEMQRQDEVNIYKTSIYILTKIGFRWAGLQQTSEEAKRNARDMDIMIDSFQGLGQTFGGGYRKAKKARARVEKFLENQIVAVREGKMKAQPGTALYEFAHWKDYKGHQMDPHLCAVELMNVVRPLAAINRFVSYVVKAMLEFDQERIKLQVADDPNYAYKFAQEVRRIFPFVPFLPGKLKKDVEFDGFKIKKGTFTLLDIFGTTHDPELFENPYQFNPDRFDNWDGSPFDLIPQGGGDFYTNHRCAGEWMTIIIMEETIKYFATKIDFDAPVQDLSVKLDQFPGKVTSGPVIHRVRPRVQR

SEQ ID NO: 103 - protein, 425 aa, Staphylococcus chromogenes
MAKKLPKDSGLDNTFKILNEAYTYVPCRLEKFGTKAFETAAIGMKPMTVISGKEAAELFYDNELMQREKTLPKRVVNTLFGKGAIHTTKGKVHVDRKALFMSLMTEENLKYLRDLTRNYWFMHTEEMQRQDEVNIYKTSIYILTKIGFRWAGLQQTSEEAKRNARDMDIMIDSFQGLGQTFGGGYRKAKKARARVEKFLENQIVAVREGKMKAQPGTALYEFAHWKDYKGHQMDPHLCAVELMNVVRPLAAINRFVSYAVKAMLEFDQERIKLQVADDPNYAYKFAQEVRRIFPFVPFLPGKLKKDVEFDGFKIKKGTFTLLDIFGTTHDSELFENPYQFNPDRFENWDGSPFDLIPQGGGDFYTNHRCAGEWMTIIIMEETIKYFATKIDFDAPVQDLSVKLDQFPGKVTSGPVIHRVRPRVQR

SEQ ID NO: 104 - protein, 425 aa, Staphylococcus chromogenes
MAKKLPKDSGLDNTFKILNEAYTYVPCRLEKFGTKAFETAAIGMKPMTVISGKEAAELFYDNELMQREKTLPKRVVNTLFGKGAIHTTKGKVHVDRKALFMSLMTEENLKYLRDLTRNYWFMHTEEMQRQDEVNIYKTSIYILTKIGFRWAGLQQTSEEAKRNARDMDIMIDSFQGLGQTFGGGYRKAKKARARVEKFLENQIVAVREGKMKAQPGTALYEFAHWKDYKGHQMDPHLCAVELMNVVRPLAAINRFVSYAVKAMLEFDQERIKLQVADDPNYAYKFAQEVRRIFPFVPFLPGKLKKDVKFDGFKIKKGTFTLLDIFGTTHDPELFENPYQFNPDRFENWDGSPFDLIPQGGGDFYTNHRCAGEWMTIIIMEETIKYFATKIDFDAPVQDLSVKLDQFPGKVTSGPVIHRVRPRVQR

SEQ ID NO: 105 - protein, 425 aa, Staphylococcus chromogenes
MAKKLPKDSGLDNTFKILNEAYTYVPCRLEKFGTKAFETAAIGMKPMTVISGKEAAELFYDNELMQREKTLPKRVVNTLFGKGAIHTTKGKVHVDRKALFMSLMTEENLKYLRDLTRNYWFMHTEEMQRQDEVNIYKTSIYILTKIGFRWAGLQQTSEEAKRNARDMDIMIDSFQGLGQTFGGGYRKAKKARARVEKFLENQIVAVREGKMKAQPGTALYEFAHWKDYKGHQMDPHLCAVELMNVVRPLAAINRFVSYVVKAMLEFDQERIKLQVADDPNYAYKFAQEVRRIFPFVPFLPGKLKKDVEFDGFKIKKGTFTLLDIFGTTHDPELFENPYQFNPDRFENWDGSPFDLIPQGGGDFYTNHRCAGEWMTIIIMEETIKYFATKIDFDAPVQDLSVKLDQFPGKVTSGPVIHRVRPRVQR

SEQ ID NO: 106 - protein, 425 aa, Staphylococcus chromogenes
MAKKLPKDSGLDNTFKILNEAYTYVPCRLEKFGTKAFETAAIGMKPMTVISGKEAAELFYDNELMQREKTLPKRVVNTLFGKGAIHTTKGKVHVDRKALFMSLMTEENLKYLRDLTRNYWFMHTEDMQRQDEVNIYKTSIYILTKIGFRWAGLQQTSEEAKRNARDMDIMIDSFQGLGQTFGGGYRKAKKARARVEKFLENQIVAVREGKMKAQPGTALYEFAHWKDYKGHQMDPHLCAVELMNVVRPLAAINRFVSYAVKAMLEFDQERIKLQVADDPNYAYKFAQEVRRIFPFVPFLPGKLKKDVEFDGFKIKKGTFTLLDIFGTTHDPELFENPYQFNPDRFENWDGSPFDLIPQGGGDFYTNHRCAGEWMTIIIMEETIKYFATKIDFDAPVQDLSVKLDQFPGKVTSGPVIHRVRPRVQR

SEQ ID NO: 107 - protein, 425 aa, Staphylococcus chromogenes
MAKKLPKDSGLDNTFKILNEAYTYVPCRLEKFGTKAFETAAIGMKPMTVISGKEAAELFYDNELMQREKTLPKRVVNTLFGKGAIHTTKGKVHVDRKALFMSLMTEENLKYLRDLTRNYWFMHTEEMQRQDEVNIYKTSIYILTKIGFRWAGLQQTSEEAKRNARDMDIMIDSFQGLGQTFGGGYRKAKKARARVEKFLENQIVAVREGKMKAQPGTALYEFAHWKDYKGHQMDPHLCAVELMNVVRPLAAINRFVSYAVKAMLEFDQERIKLQVADDPNYAYKFAQEVRRIFPFVPFLPGKLKKDVEFDGFKIKKGTFTLLDIFGTTHDPELFENPYQFNPDRFENWDGSPFDLIPQGGGDFYTNHRCAGEWMTIIIMEETIKYFATKIDFDAPVQDLSVKLDQFPGKVTSGPVIHRVRPRVQR

SEQ ID NO: 108 - protein, 425 aa, Staphylococcus chromogenes
MAKKLPKDSGLDNTFKILNEAYTYVPCRLEKFGTKAFETAAIGMKPMTVISGKEAAELFYDNELMQREKTLPKRVVNTLFGKGAIHTTKGKVHVDRKALFMSLMTEENLKYLRDLTRNYWFMHTEDMQRQDEVNIYKTSIYILTKIGFRWAGLQQTSEEAKRNARDMDIMIDSFQGLGQTFGGGYRKAKKARARVEKFLENQIVAVREGKMKAQPGTALYEFVHWKDYKGHQMDPHLCAVELMNVVRPLAAINRFVSYAVKAMLEFDQERIKLQVADDPNYAYKFAQEVRRIFPFVPFLPGKLKKDVEFDGFKIKKGTFTLLDIFGTTHDPELFENPYQFNPDRFENWDGSPFDLIPQGGGDFYTNHRCAGEWMTIIIMEETIKYFATKIDFDAPVQDLSVKLDQFPGKVTSGPVIHRVRPRVQR

SEQ ID NO: 109 - protein, 425 aa, Staphylococcus chromogenes
MAKKLPKDSGLDNTFKILNEAYTYVPCRLEKFGTKAFETAAIGMKPMTVISGKEAAELFYDNELMQREKTLPKRVVNTLFGKGAIHTTKGKVHVDRKALFMSLMTEENLKYLRDLTRNYWFMHTEEMQRQEEVNIYKTSIYILTKIGFRWAGLQQTSEEAKRNARDMDIMIDSFQGLGQTFGGGYRKAKKARARVEKFLENQIVAVREGKMKAQPGTALYEFAHWKDYKGHQMDPHLCAVELMNVVRPLAAINRFVSYAVKAMLEFDQERIKLQVADDLNYAYKFAQEVRRIFPFVPFLPGKLKKDVEFDGFKIKKGTFTLLDIFGTTHDPELFENPYQFNPDRFENWDGSPFDLIPQGGGDFYTNHRCAGEWMTIIIMEETIKYFATKIDFDAPVQDLSVKLDQFPGKVTSGPVIHRVRPRVQR

SEQ ID NO: 110 - protein, 425 aa, Staphylococcus chromogenes
MAKKLPKDSGLDNTFKILNEAYTYVPCRLEKFGTKAFETAALGMKPMTVISGKEAAELFYNNELMQREKTLPKRVVNTLFGKGAIHTTKGKVHVDRKALFMSLMTEENLKYLRDLTRNYWFMHTEEMQRQDEVNIYKTSIYILTKIGFRWAGLQQTSEEAKRNARDMDIMIDSFQGLGQTFGGGYRKAKKARARVEKFLENQIVAVREGKMKAQPGTALYEFAHWKDYKGHQMDPHLCAVELMNVVRPLAAVNRFVSYAVKAMLEFDQERIKLQVADDPNYAYKFAQEVRRIFPFVPFLPGKLKKDVEFDGFKIKKGTFTLLDIFGTTHDPELFENPYQFNPDRFENWDGSPFDLIPQGGGDFYTNHRCAGEWMTIIIMEETIKYFATKIDFDAPVQDLSVKLDQFPGKVTSGPVIHRVRPRVQR

SEQ ID NO: 111 - protein, 425 aa, Staphylococcus chromogenes
MAKKLPKDSGLDNTFKILNEAYTYVPCRLEKFGTKAFETAAIGMKPMTVISGKEAAELFYDNELMQREKTLPKRVVNTLFGKGAIHTTKGKVHVDRKALFMSLMTEENLKYLRDLTRNCWFMHTEEMQRQDEVNIYKTSIYILTKIGFRWAGLQQTSEEAKRNARDMDIMIDSFQGLGQTFGGGYRKAKKARARVEKFLENQIVAVREGKMKAQPGTALYEFAHWKDYKGHQMDPHLCAVELMNVVRPLAAINRFVSYAVKAMLEFDQERIKLQVADDPNYAYKFAQEVRRIFPFVPFLPGKLKKDVEFDGFKIKKGTFTLLDIFGTTHDPELFENPYQFNPDRFENWDGSPFDLIPQGGGDFYTNHRCAGEWMTIIIMEETIKYFATKIDFDAPVQDLSVKLDQFPGKVTSGPVIHRVRPRVQR

SEQ ID NO: 112 - protein, 425 aa, Staphylococcus chromogenes
MAKKLPKDSGLDNTFKILNEAYTYVPCRLEKFGTKAFETAAIGMKPMTVISGKEAAELFYDNELMQREKTLPKRVVNTLFGKGAIHTTKGKVHVDRKALFMSLMTEENLKYLRDLTRNYWFMHTEEMQRQDEVNIYKTSIYILTKIGFRWAGLQQTSEEAKRNARDMDIMIDSFQGLGQTFGGGYRKAKKARARVEKFLENQIVAVREGKMKAQPGTALYEFAHWKDYKGHQVDPHLCAVELMNVVRPLAAINRFVSYAVKAMLEFDQERIKLQVADDPNYAYKFAQEVRRIFPFVPFLPGKLKKDVEFDGFKIKKGTFTLLDIFGTTHDPELFENPYQFNPDRFENWDGSPFDLIPQGGGDFYTNHRCAGEWMTIIIMEETIKYFATKIDFDAPVQDLSVKLDQFPGKVTSGPVIHRVRPRVQR

SEQ ID NO: 113 - protein, 425 aa, Staphylococcus chromogenes
MAKKLPKDSGLDNTFKILNEAYTYVPCRLEKFGTKAFETAAIGMKPMTVISGKEAAELFYDNELMQREKTLPKRVVNTLFGKGAIHTTKGKVHVDRKALFMSLITEENLKYLRDLTRNYWFMHTEDMQRQDEVNIYKTSIYILTKIGFRWAGLQQTSEEAKRNARDMDIMIDSFQGLGQTFGGGYRKAKKARARVEKFLENQIVAVREGKMKAQPGTALYEFVHWKDYKGHQMDPHLCAVELMNVVRPLAAINRFVSYAVKAMLEFDQERIKLQVADDPNYAYKFAQEVRRIFPFVPFLPGKLKKDVEFDGFKIKKGTFTLLDIFGTTHDPELFENPYQFNPDRFENWDGSPFDLIPQGGGDFYTNHRCAGEWMTIIIMEETIKYFATKIDFDAPVQDLSVKLDQFPGKVTSGPVIHRVRPRVQR

SEQ ID NO: 114 - protein, 422 aa, Nosocomiicoccus sp.
MAKIKRDKGLDNTLKVFKQGYLYTTNQRERLGAEVFETRALGGKSYVVLSGKEGAELFYDNDKIEREGGLPKRVVNTLFGKGAIHTTTGKQHIDRKALFMSLMTEGNLEYVRKLTRNYWKANTARMEAMGDVNVYHESIVLLTRIGMRWAGVTAPEEEIERIAEDMDIMIDSFKGLGNAFKGYKSSKDARKRVEDWLEDQIIKTRNGEINPPKGSSLYELAHWRDYEGNQMDSRLAGIDLMNTFRPLIAINRFVSYGVLALYEHPEAIRKMSTVEDYPYMFAQEVRRFYPFVPFLPGKTKVEVEHKGVTIPKDQRLVIDVYGTLHSEELWDQPNKFIPERFRDWDGSPFDMIPQGGGDYWTNHRCAGEWITIIIMEETMKYFANEITWDVPEQDLTIDLNSIPGYINSGMVINNVREKVDRF

SEQ ID NO: 115 - protein, 422 aa, Nosocomiicoccus sp.
MAKIKRDKGLDNTLKVFKQGYLYTTNQRERLGAEVFETRALGGKSYVVLSGKEGAELFYDNDKIEREGGLPKRVVNTLFGKGAIHTTTGKQHIDRKALFMSLMTEGNLEYVRKLTRNYWKANTARMEAMGDVNVYHESIVLLTRIGMRWAGVTAPEEEIERIAEDMDIMIDSFKGLGNAFKGYKSSKDARKRVEDWLEDQIIKTRNGEINPPKGSSLYELAHWRDYEGNQMDSRLAGIDLMNTFRPLIAINRFVSYGVLALYEHPEAIRKMSTVEDYPYMFAQEVRRFYPFVPFLPGKTKVEVEHKGVTIPKDQRLVIDVYGTLHSEELWDQPNKFIPERFKDWDGSPFDMIPQGGGDYWTNHRCAGEWITIIIMEETMKYFANEITWDVPEQDLTIDLNSIPGYINSGMVINNVREKVDRF

SEQ ID NO: 116 - protein, 423 aa, Nosocomiicoccus massiliensis
MMAKIKRDKGLDNTLRVFKQGYLYTTNQRERLGAEVFETRALGGKSYVVLSGKEGAELFYDNDKIEREGGLPRRVVNTLFGKGAIHTTTGKQHIDRKALFMSLMTEGNLEYVRKLTRNYWKANTARMEAMGDVNVYHESIVLLTRIGMRWAGVTAPEEEIERIAEDMDIMIDSFKGLGNAFKGYKSSKDARKRVEDWLEDQIIKTRNGEINPPKGSSLYEFAHWRDYEGNHMDSRLAGIDLMNTFRPLIAINRFVSYGVLALYENPEAIRKMSTVEDYPYMFAQEVRRFYPFVPFLPGKTKVEVEHKGVTIPKDQRLVIDVYGTLHSEELWDQPNKFIPERFKDWDGSPFDMIPQGGGDYWTNHRCAGEWITIIIMEETMKYFANEITWDVPEQDLTIDLNSIPGYINSGMVINNVREKVDRF

SEQ ID NO: 117 - protein, 422 aa, Nosocomiicoccus massiliensis
MAKIKRDKGLDNTLKVFKQGYLYTTNQRERLGAEVFETRALGGKSYVVLSGKEGAELFYDNDKIEREGGLPKRVVNTLFGKGAIHTTTGKQHIDRKALFMSLMTEGNLEYVRKLTRNYWKANTARMEAMGDVNVYHESIVLLTRIGMRWAGVTAPEEEIERIAEDMDIMIDSFKGLGNAFKGYKSSKDARKRVEDWLEDQIIKTRNGEINPPKGSSLYELAHWRDYEGNQMDSRLAGIDLMNTFRPLIAINRFVSYGVLALYEHPEAIRKMSTVEDYPYMFAQEVRRFYPFVPFLPGKTKVEVEHKGVTIPKDQRLVIDVYGTLHSEELWNQPNKFIPERFKDWDGSPFDMIPQGGGDYWTNHRCAGEWITIIIMEETMKYFANEITWDVPEQDLTIDLNSIPGYINSGMVINNVREKVDRF

SEQ ID NO: 118 - protein, 422 aa, Nosocomiicoccus massiliensis
MAKIKRDKGLDNTLRVFKQGYLYTTNQRERLGAEVFETRALGGKSYVVLSGKEGAELFYDNDKIEREGGLPRRVVNTLFGKGAIHTTTGKQHIDRKALFMSLMTEGNLEYVRKLTRNYWKANTARMEAMGDVNVYHESIVLLTRIGMRWAGVTAPEEEIERIAEDMDIMIDSFKGLGNAFKGYKSSKDARKRVEDWLEDQIIKTRNGEINPPKGSSLYEFAHWRDYEGNHMDSRLAGIDLMNTFRPLIAINRFVSYGVLALYENPEAIRKMSTVEDYPYMFAQEVRRFYPFVPFLPGKTKVEVEHKGVTIPKDQRLVIDVYGTLHSEELWDQPNKFIPERFKDWDGSPFDMIPQGGGDYWTNHRCAGEWITIIIMEETMKYFANEITWDVPEQDLTIDLNSIPGYINSGMVINNVREKVDRF

SEQ ID NO: 119 - protein, 422 aa, Nosocomiicoccus sp.
MAKIKRDKGLDNTLKVFKQGYLYTTNQRERLGAEVFETRALGGKSYVVLSGKEGAELFYDNDKIEREGGLPKRVVNTLFGKGAIHTTTGKQHIDRKALFMSLMTEGNLEYVRKLTRNYWKANTARMEAMGDVNVYQESIVLLTRIGMRWAGVTAPEEEIERIAEDMDIMIDSFKGLGNAFKGYKSSKDARKRVEDWLEDQIIKTRNGEINPPKGSSLYELAHWRDYEGNQMDSRLAGIDLMNTFRPLIAINRFVSYGVLALYEHREAIRKMSTVEDYPYMFAQEVRRFYPFVPFLPGKTKVEVEHKGVTIPKDQRLVIDVYGTLHSEELWDQPNKFIPERFRDWDGSPFDMIPQGGGDYWTNHRCAGEWITIIIMEETMKYFANEITWDVPEQDLTIDLNSIPGYINSGMVINNVREKVDRF

SEQ ID NO: 120 - protein, 347 aa, Salinicoccus sediminis
MGTIKRDKGLDNTLKVMKQGYLYITNQRKRLGSENIFETRALGGKKIIVISGKDAAELFYDNDKIERSGTLPKRVVNTLFGKGAIHTTTGKKHIDRKALFMSLMTEGNLKHLRELTRNHWYMNTQKMEQMDQINIYRESIVQLTKIGTKWAGVQAPEEKIEEIATDMDVMIDSFKGLGGKFKGYKASVDARKRVEDWLEDQIIQTRKGKIFPPEGTALYEFAHWEDYKGNPMDSRLCAIDLMNTFRPLIAINRFVSFGLLAMHEHPITREKIKSDDDYAYMFSQEVRRFYPFVPFLPAKAKTDIQFEGHPIEKDTMIALDIYGTMHREDVFENPDDFYPERFKDWDG

SEQ ID NO: 121 - protein, 414 aa, Pontibacillus halophilus
MNVNVAKDRLDQTISLVREGYAFIPNRLHSQHLDVYETKLLGQRIALIGGEEGANLFYDSSRMKRNGALPKPVLKTLFGEGGVHTKDGAAHAHRKQLFMSLMTPDALRRLSNLTTAYWEEYAMKWEKQRKVVLYDESRELLCQVACDWAGIPLGKKEVEQRTEDLSKMIDGFSALGPKHIESRRARNRSEKWIRSIIEDVRANKLETREGSAIHEMAFHLDENGDRLDTEIAAVELLNVIRPLVAISKFLAFGAKAYADYPETREKTKESDEYILRFAQEVRRYYPFAPFLGAKVKHDFTWQGYTFRENQLVMIDLFGTNHDPRLWENPDQFDPDRFKDWKGGLFDLIPQGGGDYYDGHRCPGEWPTIEVLKASFGYLSRKIDYKVPKQDHSYSLSKMPALPKSGMILKKVRRK

SEQ ID NO: 122 - protein, 420 aa, Macrococcus sp.
MSKLKESGFDHTLTMLKEGYTYIPNRTEKYQSPIFFNRVLGGKPCAIISGKEAAELFYNNDLIERKGTMPKRIVKTLFGKGAIHTTTGKVHIDRKALFMSLMTDENLSLLRQITKDAWENEVDSLIKVNEVNVYKKSSEVLTKVGLKWAGIEDTPKNIDKIAKEMDLMIDSFGQLGGAYKGYRAAQKARANVEAYLQNQIKKVRKGKLKPRENSALYAFSHWKDMNNKPMDLALCAVDLMNVIRPLVAINRFVTYGVLAIHEFNGEKDKLKFGTDDYVYQFVQEVRRFYPFVPYLPGKAKKKLTFDNQKIKKDQMILLDVYGTLHNEKLWEYPNRFDPTRFENWDGSPFDLIPQGGGDYHTNHRCAGEWMTIIIMEETMKFFTRTISFEVPPQDFTVDLTKLPGKVASGMNIRQVQALNN

SEQ ID NO: 123 - protein, 426 aa, Staphylococcus lentus
MGKNIPRERGLDNTITVLKEAYEYVPNRLEKHNTNIFELRALGGRRTVVISGKEAAELFYNNDLIERQGTLPKRIVNTLFGKGAIHTTGGKKHIDRKALFMSLMTEENLEYLRELTRNHWFMNTERMERMDEVNVYKESILMLTKIGFRWAGIIASPEEIEQCAADMDTMIDSFKNIGTVFKGYREAKQARDRVETFLENQIIAVREGKINPPQGTALYEFSHWEDFEGNLMDSRLCSIDLMNVVRPLVAINRFVSFGVKAFHDYPGEAERVFNNEDDYAYKFIQEVRRFYPFVPYLPGKAAVDIDFEGYTIEKDTFLVLDIYGTLHRDGLWENPERFYPNRFNDWDGSPFDLIPQGGGDYYTNHRCAGEWMTIIIMEETMKYFAQNISYDVKKDQDTSVNLNKLPGRVVDGMIITNVNALVNRNV

SEQ ID NO: 124 - protein, 426 aa, Staphylococcus lentus
MGKNIPRERGLDNTITVLKEAYEYVPNRLEKHNTNIFELRALGGRRTVVISGKEAAELFYNNDLIERQGTLPKRIVNTLFGKGAIHTTGGKKHIDRKALFMSLMTEENLEYLRELTRNHWFMNTERMERMDEVNVYKESILMLTKIGFRWAGIIASPEEIEQCAADMDTMIDSFKNIGTVFKGYREAKQARDRVETFLENQIIAVREGKINPPQGTALYEFSHWEDFEGNLMDSRLCSIDLMNVVRPLVAINRFVSFGVKAFHDYPGEAERVFNNEDDYAYKFIQEVRRFYPFVPYLPGKAAVDIDFEGYTIEKDTFLVLDIYGTLHREGLWENPERFYPNRFNDWDGSPFDLIPQGGGDYYTNHRCAGEWMTIIIMEETMKYFAQNISYDVKKDQDTSVNLNKLPGRVVDGMIITNVNALVNRNV

SEQ ID NO: 125 - protein, 426 aa, Staphylococcus lentus
MGKNIPRERGLDNTITVLKEAYEYVPNRLEKHDTNIFELRALGGRRTVVISGKEAAELFYNNDLIERQGTLPKRIVNTLFGKGAIHTTGGKKHIDRKALFMSLMTEENLEYLRELTRNHWFMNTERMERMDEVNVYKESILMLTKIGFRWAGIIASPEEIEQCAADMDTMIDSFKNIGTVFKGYREAKQARDRVETFLENQIIAVREGKINPPQGTALYEFSHWEDFEGNLMDSRLCSIDLMNVVRPLVAINRFVSFGVKAFHDYPGEAERVFNNEDDYAYKFIQEVRRFYPFVPYLPGKAAVDIDFEGYTIEKDTFLVLDIYGTLHREGLWENPERFYPNRFNDWDGSPFDLIPQGGGDYYTNHRCAGEWMTIIIMEETMKYFAQNISYDVKKDQDTSVNLNKLPGRVVDGMIITNVNALVNRNV

SEQ ID NO: 126 - protein, 422 aa, Staphylococcus pseudintermedius
MAKKLPKDTGLDNTLKMINEAYTYVPKRLEKFGTKAFETRALGMKPIVVISGKAAAELFYDNDKISRKGTLPKRIVHTLFGKGAIHTTEGKVHVDRKALFMSLMTEENLKYLRELTRNYWFMHTERMQNKDEVNVYQEAGLILTKVGFRWAGLKQTDEQAAQNAEDMNTMIDSFSGLGQSLKGYREAKKARARVEQFLQEQIEAVRVGQQYAEPGTALYEFAHWKDLNDQPMDPHLCAVDLMNIVRPLVAVNRFVSYGVKALIEFDQERKKLQVTNDPNYAYKFAQEVRRIFPFVPFLPGRLKKTVEFDGFKLKKGTLTVLDIFGTTHDPELFENPYQFNPDRFDNWDGSPFDLIPQGGGDFYTNHRCAGEWMTVIVMEETIQYFANKIDFVVPAQDLSVKLSQFPGKVTSGTMIKNVYPRI

SEQ ID NO: 127 - protein, 422 aa, Staphylococcus pseudintermedius
MAKKLPKDTGLDNTLKMINEAYTYVPKRLEKFGTKAFETRALGMKPIVVISGKAAAELFYDNDKISRKGTLPKRIVHTLFGKGAIHTTEGKVHVDRKALFMSLMTEENLKYLRELTRNYWFMHTERMQNKDEVNVYQEAGLILTKVGFRWAGLKQTDEQAAQNAEDMNTMIDSFSGLGQSLKGYREAKKARARVEQFLQEQIEAVRVGQQYAEPGTALYEFAHWKDLNDQPMDPHLCAVDLMNIVRPLVAVNRFVSYGVKALIEFDQERKKLQVTNDPNYAYKFAQEVRRIFPFVPFLPGRLKKTVEFDGFKLKKGTLTVLDIFGTTHDPELFENPYQFNPDRFDNWDGSPFDLIPQGGGDFYTNHRCAGEWMTVIVMEETIQYFANKIDFVAPPQDLSVKLSQFPGKVTSGTMIKNVYPRI

SEQ ID NO: 128 - protein, 422 aa, Staphylococcus pseudintermedius
MAKKLPKDTGLDNTLKMINEAYTYVPKRLEKFGTKAFETRALGMKPIVVISGKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLKQTDEQVAQNAEDMNTMIDSFSGLGQSLKGYREAKKARARVEQFLQEQIEAVRVGQQYAEPGTALYEFAHWKDLNDQPMDPHLCAVDLMNIVRPLVAVNRFVSYGVKALIEFDQERKKLQVTNDPNYAYKFAQEVRRIFPFVPFLPGRLKKTVEFDGFKLKKGTLTVLDIFGTTHDPELFENPYQFNPDRFDNWDGSPFDLIPQGGGDFYTNHRCAGEWMTVIVMEETIQYFANKIDFVVPAQDLSVKLSQFPGKVTSGTMIKNVYPRI

SEQ ID NO: 129 - protein, 426 aa, Macrococcus brunensis
MAKRMPKVKGLDQTLTVLKEGYEFIPNRTKELNTNVFEMRVLGGKKAVVISGKEAAELFYDNDKTERSGTLPKRLVKTLFGKGAIHTTTGDKHKNRKTLFMSLMTDNNLEYLRKLTRNYWFEDTARLQAMDQTNVYLESTLVLLKVGFRFADVPFDTPERMEQYAKDMNAMVDSFGSIGTAYSGYKRALNARDRVEDYLEKQIIATRKGKLFPEEGSGLYEFSHWKDMNGKQMDSRLAAVDLMNTIRPLVAINRFISFGVLAMHEYPGERARVAQNDNSYAYKFAQEVRRYYPFVPFLPGKAKKNITFDGYKIEKDTMLILDIFGTLHSESLWQNAEQFYPERFENWDGSPFDLIPQGGGDYWTNHRCAGEWMTVIIMEESMKYFAQEIQYDVPAQDFTVDRTKLPGQINSGMIIENIRNNFDRRR

SEQ ID NO: 130 - protein, 422 aa, Staphylococcus pseudintermedius
MAKKLPKDTGLDNTLKMINEAYTYVPKRLEKFGTKAFETRALGMKPIVVISGKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLKQTDEQAVQNAEDMNTMIDSFSGLGQSLKGYREAKKARARVEQFLQEQIEAVRVGQQYAEPGTALYEFAHWKDLNDQPMDPHLCAVDLMNIVRPLVAVNRFVSYGVKALTEFDQERKKLQVTNDPNYAYKFAQEVRRIFPFVPFLPGRLKKTVEFDGFKLKKGTLTVLDIFGTTHDPELFENPYQFNPDRFDNWDGSPFDLIPQGGGDFYTNHRCAGEWMTVIVMEETIQYFANKIDFVVPAQDLSVKLSQFPGKVTSGTMIKNVYPRI

SEQ ID NO: 131 - protein, 422 aa, Staphylococcus pseudintermedius
MAKKLPKDTGLDNTLKMIYEAYTYVPKRLEKFGTKAFETRALGMKPIVVISGKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLKQTDEQAVQNAEDMNTMIDSFSGLGQSLKGYREAKKARARVEQFLQEQIEAVRVGQQYAEPGTALYEFAHWKDLNDQPMDPHLCAVDLMNIVRPLVAVNRFVSYGVKALIEFDQERKKLQVTNDPNYAYKFAQEVRRIFPFVPFLPGRLKKTVEFDGFKLKKGTLTVLDIFGTTHDPELFENPYQFNPDRFDNWDGSPFDLIPQGGGDFYTNHRCAGEWMTVIVMEETIQYFANKIDFVVPAQDLSVKLSQFPGKVTSGTMIKNVYPRI

SEQ ID NO: 132 - protein, 422 aa, Staphylococcus pseudintermedius
MAKKLPKDTGLDNTLKMINEAYTYVPKRLEKFGTKAFETRALGMKPIVVISGKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLRQTDEQAVQNAEDMNTMIDSFSGLGQSLKGYREAKKARARVEQFLQEQIEAVRVGQQYAEPGTALYEFAHWKDLNDQPMDPHLCAVDLMNIVRPLVAVNRFVSYGVKALIEFDQERKKLQVTNDPNYAYKFAQEVRRIFPFVPFLPGRLKKTVEFDGFKLKKGTLTVLDIFGTTHDPELFENPYQFNPDRFDNWDGSPFDLIPQGGGDFYTNHRCAGEWMTVIVMEETIQYFANKIDFVVPAQDLSVKLSQFPGKVTSGTMIKNVYPRI

SEQ ID NO: 133 - protein, 422 aa, Staphylococcus sp.
MAKKLPKDTGLDNTLKMINEAYTYVPKRLEKFGTKAFETRALGMKPIVVISGKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLKQTDEQAVQNAEDMNTMIDSFSGLGQSLKGYREAKKARARVEQFLQEQIEAVRVGQQYAEPGTALYEFAHWKDLNDQPMDPHLCAVDLMNIVRPLVAVNRFVSYGVKALIEFDQERKKLQVTNDPNYAYKFAQEVRRIFPFVPFLPGRLKKTVEFDGFKLKKGTLTVLDIFGTTHDPELFENPYQFNPDRFDNWDGSPFDLIPQGGGDFYTNHRCAGEWMTVIVMEETIQYFANKIDFVVPAQDLSVKLSQFPGKVTSGTMIKNVYPRI

SEQ ID NO: 134 - protein, 422 aa, Staphylococcus pseudintermedius
MAKKLPKDTGLDNTLKMINEAYTYVPKRLEKFGTKAFETRALGMKPIVVISGKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLKQTDEQAAQNAEDMNTMIDSFSGLGQSLKGYREAKKARARVEQFLQEQIEAVRVGQQYAEPGTALYEFAHWKDLNDQPMDPHLCAVDLMNIVRPLVAVNRFVSYGVKALIEFDQERKKLQVTNDPNYAYKFAQEVRRIFPFVPFLPGRLKKTVEFDGFKLKKGTLTVLDIFGTTHDPELFENPYQFNPDRFDNWDGSPFDLIPQGGGDFYTNHRCAGEWMTVIVMEETIQYFANKIDFVVPAQDLSVKLSQFPGKVTSGTMIKNVYPRI

SEQ ID NO: 135 - protein, 422 aa, Staphylococcus pseudintermedius
MAKKLPKDTGLDNTLKMINEAYTYVPKRLEKFGTKAFETRALGMKPIVVISGKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLKQTDEQAAQNAEDMNTMIDSFSGLGQSLKGYREAKKARARVEQFLQEQIEAVRVGQQYAEPGTALYEFAHWKDLNDQPMDPHLCAVDLMNIVRPLVAVNRFVSYGVKALIEFDQERKKLQVTNDPNYAYKFAQEVRRIFPFVPFLPGRLKKTVEFDGFKLKKGTLTVLDIFGTTHDPELFENPYQFNPDRFDNWDGSPFDLIPQGGGDFYTNHRCAGEWMTVIVMEETIQYFANKIDFVAPPQDLSVKLSQFPGKVTSGTMIKNVYPRI

SEQ ID NO: 136 - protein, 422 aa, Staphylococcus pseudintermedius
MAKKLPKDTGLDNTLKMINEAYTYVPKRLEKFGTKAFETRALGMKPIVVISGKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLKQTDEQAAQNAEDMNTMIDSFSGLGQSLKGYREAKKARARVEQFLQEQIEAVRVGQQYAEPGTALYEFAHWKDLNDQPMDPHLCAVDLMNIVRPLVAVNRFVSYGVKALIEFDQERKKLQVTNDPNYAYKFSQEVRRIFPFVPFLPGRLKKTVEFDGFKLKKGTLTVLDIFGTTHDPELFENPYQFNPDRFDNWDGSPFDLIPQGGGDFYTNHRCAGEWMTVIVMEETIQYFANKIDFVVPAQDLSVKLSQFPGKVTSGTMIKNVYPRI

SEQ ID NO: 137 - protein, 422 aa, Staphylococcus pseudintermedius
MAKKLPKDTGLDNTLKMINEAYTYVPKRLEKFGTKAFETRALGMKPIVVISGKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLKQTDEQAVQNAEDMNTMIDSFSGLGQSLKGYREAKKARARVEQFLQEQIEAVRVGQQYAEPGTALYEFAHWKDLNDQPMDPHLCAVDLMNIVRPLVAVNRFVSYGVKALIEFDQERKKLQVTNDPNYAYKFSQEVRRIFPFVPFLPGRLKKTVEFDGFKLKKGTLTVLDIFGTTHDPELFENPYQFNPDRFDNWDGSPFDLIPQGGGDFYTNHRCAGEWMTVIVMEETIQYFANKIDFVVPAQDLSVKLSQFPGKVTSGTMIKNVYPRI

SEQ ID NO: 138 - protein, 422 aa, Staphylococcus pseudintermedius
MAKKLPKDTGLDNTLKMINEAYTYVPKRLEKFGTKAFETRALGMKPIVVISGKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLRQTDEQAVQNAEDMNTMIDSFSGLGQSLKGYREAKKARARVEQFLQEQIEAVRVGQQYAEPGTALYEFAHWKDLNDQPMDPHLCAVDLMNIVRPLVAVNRFVSYGVKALIEFDQERKKLQVTNDPNYAYKFAQEVRRIFPFVPFLPGRLKKTVEFDGFKLKKGTLTVLDILGTTHDPELFENPYQFNPDRFDNWDGSPFDLIPQGGGDFYTNHRCAGEWMTVIVMEETIQYFANKIDFVAPPQDLSVKLSQFPGKVTSGTMIKNVYPRI

SEQ ID NO: 139 - protein, 422 aa, Staphylococcus pseudintermedius
MAKKLPKDTGLDNTLKMINEAYTYVPKRLEKFGTKAFETRALGMKPIVVISGKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLRQTDEQAVQNAEDMNTMIDSFSGLGQSLKGYREAKKARARVEQFLQEQIEAVRVGQQYAEPGTALYEFAHWKDLNDQPMDPHLCAVDLMNIVRPLVAVNRFVSYGVKALIEFDQERKKLQVTNDPNYAYKFAQEVRRIFPFVPFLPGRLKKTVEFDGFKLKKGTLTVLDIFGTTHDPELFENPYQFNPDRFDNWDGSPFDLIPQGGGDFYTNHRCAGEWMTVIVMEETIQYFANKIDFVAPPQDLSVKLSQFPGKVTSGTMIKNVYPRI

SEQ ID NO: 140 - protein, 422 aa, Staphylococcus pseudintermedius
MAKKLPKDTGLDNTLKIINEAYTYVPKRLEKFGTKAFETRALGMKPIVVISGKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLKQTDEQAAQNAEDMNTMIDSFSGLGQSLKGYREAKKARARVEQFLQEQIEAVRVGQQYAEPGTALYEFAHWKDLNDQPMDPHLCAVDLMNIVRPLVAVNRFVSYGVKALIEFDQERKKLQVTNDPNYAYKFAQEVRRIFPFVPFLPGRLKKTVEFDGFKLKKGTLTVLDIFGTTHDPELFENPYQFNPDRFDNWDGSPFDLIPQGGGDFYTNHRCAGEWMTVIVMEETIQYFANKIDFVAPAQDLSVKLSQFPGKVTSGTMIKNVYPRI

SEQ ID NO: 141 - protein, 422 aa, Staphylococcus pseudintermedius
MAKKLPKDTGLDNTLKMINEAYTYVPKRLEKFGTKAFETRALGMKTIVVISGKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLKQTDEQAAQNAEDMNTMIDSFSGLGQSLKGYREAKKARARVEQFLQEQIEAVRVGQQYAEPGTALYEFAHWKDLNDQPMDPHLCAVDLMNIVRPLVAVNRFVSYGVKALIEFDQERKKLQVTNDPNYAYKFAQEVRRIFPFVPFLPGRLKKTVEFDGFKLKKGTLTVLDIFGTTHDPELFENPYQFNPDRFDNWDGSPFDLIPQGGGDFYTNHRCAGEWMTVIVMEETIQYFANKIDFVAPAQDLSVKLSQFPGKVTSGTMIKNVYPRI

SEQ ID NO: 142 - protein, 422 aa, Staphylococcus pseudintermedius
MAKKLPKDTGLDNTLKMINEAYTYVPKRLEKFGTKAFETRALGMKPIVVISGKAAAELFYDNDKISRKGTLPKRIVHTLFGKGAIHTTEGKVHVDRKALFMSLMTEKNLKYLRELTRNYWFMHTERMQNKDEVNVYQETGLILTKVGFRWAGLKQTDEQAAQNAEDMNTMIDSFSGLGQSLKGYREAKKARARVEQFLQEQIEAVRVGQQYAEPGTALYEFAHWKDLNDQPMDPHLCAVDLMNIVRPLVAVNRFVSYGVKALIEFDQERKKLQVTNDPNYAYKFAQEVRRIFPFVPFLPGRLKKTVEFDGFKLKKGTLTVLDIFGTTHDPELFENPYQFNPDRFDNWDGSPFDLIPQGGGDFYTNHRCAGEWMTVIVMEETIQYFANKIDFVVPAQDLSVKLSQFPGKVTSGTMIKNVYPRI

SEQ ID NO: 143 - protein, 422 aa, Staphylococcus pseudintermedius
MAKKLPKDTGLDNTLKMINEAYTYVPKRLEKFGTKAFETRALGMKPIVVISGKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLKQTDEQAAQNAEDMNTMIDSFSGLGQSLKGYREAKKARARVEQFLQEQIEAVRVGQQYAEPGTALYEFAHWKDLNDQPMDPHLCAVDLMNIVRPLVAVNRFVSYGVKALIEFDQERKKLQVTNDPNYAYKFAQEVRRIFPFVPFLPGRLKKTVEFDGFKLKKGTLTVLDIFGTTHDPELFENPYQLNPDRFDNWDGSPFDLIPQGGGDFYTNHRCAGEWMTVIVMEETIQYFANKIDFVVPAQDLSVKLSQFPGKVTSGTMIKNVYPRI

SEQ ID NO: 144 - protein, 422 aa, Staphylococcus pseudintermedius
MANKLPKDTGLDNTLKMINEAYTYVPKRLEKFGTKAFETRALGMKPIVVISGKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLRQTDEQAVQNAEDMNTMIDSFSGLGQSLKGYREAKKARARVEQFLQEQIEAVRVGQQYAEPGTALYEFAHWKDLNDQPMDPHLCAVDLMNIVRPLVAVNRFVSYGVKALIEFDQERKKLQVTNDPNYAYKFAQEVRRIFPFVPFLPGRLKKTVEFDGFKLKKGTLTVLDIFGTTHDPELFENPYQFNPDRFDNWDGSPFDLIPQGGGDFYTNHRCAGEWMTVIVMEETIQYFANKIDFVAPPQDLSVKLSQFPGKVTSGTMIKNVYPRI

SEQ ID NO: 145 - protein, 422 aa, Staphylococcus pseudintermedius
MAKKLPKDTGLDNTLKMINEAYTYVPKRLEKFGTKAFETRALGMKPIVVISGKAAAELFYDNDKISRKGTLPKRIVHTLFGKGAIHTTEGKVHVDRKALFMSLMTEKNLKYLCELTRNYWFMHTERMQNKDEVNVYQEAGLILTKVGFRWAGLRQTDEQAVQNAEDMNTMIDSFSGLGQSLKGYREAKKARARVEQFLQEQIEAVRVGQQYAEPGTALYEFAHWKDLNDQPMDPHLCAVDLMNIVRPLVAVNRFVSYGVKALIEFDQERKKLQVTNDPNYAYKFAQEVRRIFPFVPFLPGRLKKTVEFDGFKLKKGTLTVLDIFGTTHDPELFENPYQFNPDRFDNWDGSPFDLIPQGGGDFYTNHRCAGEWMTVIVMEETIQYFANKIDFVVPAQDLSVKLSQFPGKVTSGTMIKNVYPRI

SEQ ID NO: 146 - protein, 371 aa, Staphylococcus pseudintermedius
GKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLRQTDEQAVQNAEDMNTMIDSFSGLGQSLKGYREAKKARARVEQFLQEQIEAVRVGQQYAEPGTALYEFAHWKDLNDQPMDPHLCAVDLMNIVRPLVAVNRFVSYGVKALIEFDQERKKLQVTNDPNYAYKFAQEVRRIFPFVPFLPGRLKKTVEFDGFKLKKGTLTVLDILGTTHDPELFENPYQFNPDRFDNWDGSPFDLIPQGGGDFYTNHRCAGEWMTVIVMEETIQYFANKIDFVAPPQDLSVKLSQFPGKVTSGTMIKNVYPRI

SEQ ID NO: 147 - protein, 352 aa, Staphylococcus pseudintermedius
MAKKLPKDTGLDNTLKMINEAYTYVPKRLEKFGTKAFETRALGMKPIVVISGKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLKQTDEQAAQNAEDMNTMIDSFSGLGQSLKGYREAKKARARVEQFLQEQIEAVRVGQQYAEPGTALYEFAHWKDLNDQPMDPHLCAVDLMNIVRPLVAVNRFVSYGVKALIEFDQERKKLQVTNDPNYAYKFAQEVRRIFPFVPFLPGRLKKTVEFDGFKLKKGTLTVLDIFGTTHDPELFENPYQFNPDRFDNWDGSPF

SEQ ID NO: 148 - protein, 423 aa, Jeotgalicoccus saudimassiliensis
MMASLKRDKGLDNTLKLMKQGYLYTTNQRNRLDTTVFETKVLGGKPFAVVTGKEGAEMFYNNDVVQREGMLPKRIVNTLFGKGAIHTIDGKKHIDRKALFMSLMTEGNLDYVRELTRTLWRANTQRMESMDQVNIYRESIVLLTKVGTRWAGVQAPPEDIERIATDMDIMIDSFKGLGGAFKGYKESKAARRRVEDWLEEQILETRKGNIHPPEGTALYEFAHWEDYLGNPMDSRNCAIDLMNTFRPLIAINRFVSFGLLAMHDNPITREKIKSEPDYAYKFAQEVRRFYPFVPFLPGKAKVDIDFQGVTIPAGHGLALDVYGTMHDETLWEDPNEFRPERFEDWDGSPFDLIPQGGGDYWTNHRCAGEWITVIIMEETMKYFAEKVTYDVPEQDLTVDLNSIPGYVKSGFIISNVQEVVDRK

SEQ ID NO: 149 - protein, 423 aa, Jeotgalicoccus sp.
MMASLKRDKGLDNTLKLMKQGYLYTTNQRNRLNTTVFETKVLGGKPFAVVTGKEGAEMFYNNDIVQREGMLPKRIVNTLFGKGAIHTIDGKKHIDRKALFMSLMTEGNLNYVRELTRTLWQANTQRMESMDQVNIYRESIVLLTKVGTRWAGVQAPPEDIERIATDMDIMIDSFKGLGGVFKGYKESKAARRRVEDWLEEQILETRKGNIHPPEGTALYEFAHWEDYLGNPMDSRNCAIDLMNTFRPLIAINRFVSFGLLAMHDNPITREKIKSEPDYAYKFAQEVRRFYPFVPFLPGKAKVDIDFQGVTIPAGHGLAIDVYGTMHDETLWENPNEFRPERFEDWDGSPFDLIPQGGGDYWTNHRCAGEWITVIIMEETMKYFAEKVTYDVPEQDLTVDLNSIPGYVKSGFIISNVQEVVDRK

SEQ ID NO: 150 - protein, 422 aa, Jeotgalicoccus saudimassiliensis
MASLKRDKGLDNTLKLMKQGYLYTTNQRNRLDTTVFETKVLGGKPFAVVTGKEGAEMFYNNDVVQREGMLPKRIVNTLFGKGAIHTIDGKKHIDRKALFMSLMTEGNLDYVRELTRTLWRANTQRMESMDQVNIYRESIVLLTKVGTRWAGVQAPPEDIERIATDMDIMIDSFKGLGGAFKGYKESKAARRRVEDWLEEQILETRKGNIHPPEGTALYEFAHWEDYLGNPMDSRNCAIDLMNTFRPLIAINRFVSFGLLAMHDNPITREKIKSEPDYAYKFAQEVRRFYPFVPFLPGKAKVDIDFQGVTIPAGHGLALDVYGTMHDETLWEDPNEFRPERFEDWDGSPFDLIPQGGGDYWTNHRCAGEWITVIIMEETMKYFAEKVTYDVPEQDLTVDLNSIPGYVKSGFIISNVQEVVDRK

SEQ ID NO: 151 - protein, 441 aa, Macrococcus caseolyticus
MRVEFTINYINVEGISMSKRVPKDRGIDNSLKIMKEGYEYVPARMKKFNTNIFETRVLGGKTAVVISGKDAAELFYDNDKTERKGTLPKRVVKTLFGKGAIHTTTGKKHIDRKALFMSLMTDENLAYLRKLTRIYWFQNIEHMQYKQKVNVYEEATELLTKVGLRWAGIVDHPENIQKMADDMNKMIDSFSAIGSLYGGYREAKKARARVEQFLEDQITAVRKGKIHPEKGTALYEFSHWEDMNGKPMDARLCAVDLMNVIRPLVAINKFVSFGVLALHEFPGERVRVALNEGDYAYKFVQEVRRYYPFVPFLPGKAKENITFDGYKIQKDTMMLLDIYGTLHRDDLFSEPERFNPYRFDNWDGSPFDLIPQGGGDYYTNHRCAGEWMTIIIMEETMKFFANEISYDVPPQDFTVDTTKFPGKVASGMDIENIRVNIDRTK

SEQ ID NO: 152 - DNA, 1266 bp, Artificial Sequence
  Artificial codon optimized gene sequence
atggcgaccctgaagcgtgacaaaggcctggataacaccctgaaggtgctgaaacagggttacctgtataccaccaaccaacgtaaccgtctgaacaccagcgttttccagaccaaagcgctgggtggcaagccgtttgtggttgtgaccggcaaagagggtgcggaaatgttctacaacaacgacgttgtgcaacgtgagggcatgctgccgaaacgtatcgtgaacaccctgttcggcaagggtgcgattcacaccgttgacggcaagaaacacgtggatcgtaaggcgctgtttatgagcctgatgaccgagggtaacctgaactacgttcgtgaactgacccgtaccctgtggcacgcgaacacccagcgtatggagagcatggacgaagtgaacatctatcgtgaaagcattgttctgctgaccaaagtgggcacccgttgggcgggtgttcaagctccgccggaggatatcgaacgtattgcgaccgacatggatatcatgattgacagcttccgtgcgctgggtggcgcgtttaagggttataaagcgagcaaagaggcgcgtcgtcgtgtggaagattggctggaggaacagatcattgagacccgtaagggcaacatccacccgccggaaggtaccgcgctgtacgagttcgcgcactgggaagactatctgggcaacccgatggatagccgtacctgcgcgattgacctgatgaacacctttcgtccgctgatcgcgattaaccgtttcgtgagctttggtctgcacgcgatgaacgagaacccgatcacccgtgagaagattaaaagcgaaccggactacgcgtataaattcgcgcaggaagtgcgtcgttactatccgttcgttccgtttctgccgggcaaggcgaaagttgacatcgattttcaaggtgttaccatcccggcgggcgtgggtctggcgctggatgtttacggtaccacccacgacgagagcctgtgggacgatccgaacgaattccgtccggagcgttttgaaacctgggacggcagcccgttcgatctgatcccgcagggtggcggtgattattggaccaaccaccgttgcgcgggcgagtggatcaccgtgatcattatggaggaaaccatgaaatacttcgcggaaaagattacctatgacgtgccggagcaagacctggaagttgatctgaacagcatcccgggctacgttaaaagcggttttgttattaagaacgtgcgtgaggttgtggatcgtacc

SEQ ID NO: 153 - protein, 436 aa, Jeotgalicoccus sp.
MHHHHHHENLYFQGMATLKRDKGLDNTLKVLKQGYLYTTNQRNRLNTSVFQTKALGGKPFVVVTGKEGAEMFYNNDVVQREGMLPKRIVNTLFGKGAIHTVDGKKHVDRKALFMSLMTEGNLNYVRELTRTLWHANTQRMESMDEVNIYRESIVLLTKVGTRWAGVQAPPEDIERIATDMDIMIDSFRALGGAFKGYKASKEARRRVEDWLEEQIIETRKGNIHPPEGTALYEFAHWEDYLGNPMDSRTCAIDLMNTFRPLIAINRFVSFGLHAMNENPITREKIKSEPDYAYKFAQEVRRYYPFVPFLPGKAKVDIDFQGVTIPAGVGLALDVYGTTHDESLWDDPNEFRPERFETWDGSPFDLIPQGGGDYWTNHRCAGEWITVIIMEETMKYFAEKITYDVPEQDLEVDLNSIPGYVKSGFVIKNVREVVDRT

SEQ ID NO: 154 - DNA, 1308 bp, Artificial Sequence
  Artificial codon optimized gene sequence
catatgcaccaccaccaccaccactcaaaacgcgttcccaaagatagagggattgataacagcctgaagatcatgaaagagggttacgaatatgttccggcgcgtatgaagaagttcaacaccaacatcttcgagacccgtgtgctgggtggcaagaccgcggtggttatcagcggcaaagatgcggcggagctgttctacgacaacgataagaccgaacgtaaaggcaccctgccgaagcgtgtggttaaaaccctgttcggcaagggtgcgattcacaccaccaccggtaagaaacacatcgatcgtaaagcgctgtttatgagcctgatgaccgacgagaacctggcgtacctgcgtaagctgacccgtatttattggtttcagaacatcgagcacatgcagtacaagcaaaaagtgaacgtttatgaggaagcgaccgaactgctgaccaaagttggcctgcgttgggcgggtattgtggatcacccggaaaacatccaaaagatggcggacgatatgaacaaaatgattgacagcttcagcgcgatcggcagcctgtacggtggctatcgtgaggcgaagaaagcgcgtgcgcgtgttgagcagtttctggaagaccaaattaccgcggtgcgtaagggcaaaatccacccggaaaagggcaccgcgctgtacgagttcagccactgggaagacatgaacggtaaaccgatggatgcgcgtctgtgcgcggtggacctgatgaacgttattcgtccgctggttgcgatcaacaagttcgtgagctttggcgttctggcgctgcacgagttcccgggtgaacgtgtgcgtgttgcgctgaacgagggcgattacgcgtataaatttgtgcaggaagttcgtcgttactatccgttcgtgccgtttctgccgggcaaggcgaaagagaacattaccttcgacggttacaagatccaaaaagacaccatgatgctgctggatatttatggcaccctgcaccgtgacgacctgttcagcgagccggaacgtttcaacccgtaccgttttgacaactgggatggcagcccgtttgatctgatcccgcagggtggcggtgactactataccaaccaccgttgcgcgggcgagtggatgaccatcattatcatggaggaaaccatgaagttctttgcgaacgaaatcagctatgacgttccgccgcaagacttcaccgtggataccaccaagtttccgggcaaagttgcgagcggtatggatattgaaaacatccgtgtgaacatcgaccgtaccaaataatgaaagctt

SEQ ID NO: 155 - protein, 431 aa, Micrococcus lylae
MHHHHHHSKRVPKDRGIDNSLKIMKEGYEYVPARMKKFNTNIFETRVLGGKTAVVISGKDAAELFYDNDKTERKGTLPKRVVKTLFGKGAIHTTTGKKHIDRKALFMSLMTDENLAYLRKLTRIYWFQNIEHMQYKQKVNVYEEATELLTKVGLRWAGIVDHPENIQKMADDMNKMIDSFSAIGSLYGGYREAKKARARVEQFLEDQITAVRKGKIHPEKGTALYEFSHWEDMNGKPMDARLCAVDLMNVIRPLVAINKFVSFGVLALHEFPGERVRVALNEGDYAYKFVQEVRRYYPFVPFLPGKAKENITFDGYKIQKDTMMLLDIYGTLHRDDLFSEPERFNPYRFDNWDGSPFDLIPQGGGDYYTNHRCAGEWMTIIIMEETMKFFANEISYDVPPQDFTVDTTKFPGKVASGMDIENIRVNIDRTK

SEQ ID NO: 156 - protein, 453 aa, Staphylococcus massiliensis
MFVDSILVLRLNLLKTGIQLEMKNGGIKVAKKLPKVKGLDNTVDIIKGGYTYVPGKLEEFDSKAFEVRALGGKKIAVMSGKEAAEIFYDNEKMERQGTLPKRIVNTLFGKGAIHTTAGKKHVDRKALFMSLMTDENLNYLRELTRNYWFMNTERMQSMDKVNVYNESIYMLTKIGFRWAGIIQTPEEAEQNAKDMDTMINSFVSLGSAYKGYKKAKKARKRVEDFLEKQIIDVRKGKLHPEEGTALYEFAHWEDLNDNPMDSHLCAVDLMNVVRPLAAINRFISYGVKVLIEFDQEKEKLRLENNEDYAYKFAQEVRRIFPFVPYLPGRAAVDLEYDGYKIPAGMMTALDVYGTTHDEDLWENPDQFNPNRFDNWDGSPFDLIPQGGGDFYTNHRCAGEWITVIIMEETMKYFANKIEFDVPSQDLSVKLDKLPGNVTSGTIISNVRPRVARK

SEQ ID NO: 157 - protein, 425 aa, Staphylococcus massiliensis
MAKKLPKVKGLDNTVDIIKGGYTYVPGKLEEFDSKAFEVRALGGKKIAVMSGKEAAEIFYDNEKMERQGTLPKRIVNTLFGKGAIHTTAGKKHVDRKALFMSLMTDENLNYLRELTRNYWFMNTERMQSMDKVNVYNESIYMLTKIGFRWAGIIQTPEEAEQNAKDMDTMINSFVSLGSAYKGYKKAKKARKRVEDFLEKQIIDVRKGKLHPEEGTALYEFAHWEDLNDNPMDSHLCAVDLMNVVRPLAAINRFISYGVKVLIEFDQEKEKLRLENNEDYAYKFAQEVRRIFPFVPYLPGRAAVDLEYDGYKIPAGMMTALDVYGTTHDEDLWENPDQFNPNRFDNWDGSPFDLIPQGGGDFYTNHRCAGEWITVIIMEETMKYFANKIEFDVPSQDLSVKLDKLPGNVTSGTIISNVRPRVARK

SEQ ID NO: 158 - protein, 422 aa, Jeotgalicoccus halophilus
MASLKRDKGLDNTLKVMKQGYLYTTNQRNRLNTTVFETKALGGKPFAVVTGKEGAEMFYNNDIVQREGMLPKRIVNTLFGKGAIHTIDGKKHVDRKALFMSLMTEGNLNYVRELTRTLWQANTQRMESMDEVNIYRESIVLLTKVGTRWAGVQAPPEQIERIATDMDIMIDSFKGLGGVFKGYKESKAARRRVEDWLEDQILETRKGNIHPPEGTALYEFAHWEDYLGNPMDSRNCAIDLMNTFRPLIAINRFVSFGLLAMHDNPVSREKIKSEPDYAYKFAQEVRRYYPFVPFLPGKAKTDIDFQGVTIPAGHGLAIDVYGTLHDESLWEDPNEFRPERFEGWDGSPFDLIPQGGGDYWTNHRCAGEWITVIIMEETMKYFAEKVTYDVPEQDLTVDLNSIPGYVKSGFVIKNVQEVVDRR

SEQ ID NO: 159 - DNA, 1332 bp, Artificial Sequence
  Artificial codon optimized gene sequence
catatgcatcaccaccaccaccacgagaacctgtacttccagggcatggcgaagcgtatgccgaaggtgaaaggtctggaccaaaccctgaccgttctgaaagagggttatgaattcattccgaaccgtacccgtaaactgaacaccaacgtttttgagattcgtatcctgggtggcaagaaagcggtggttatcagcggcaaagaggcggcggaactgttttacgacaacgataaaaccgaacgtgcgggtaccctgccgaagcgtctggtgaaaaccctgttcggcaagggtgcgattcacaccaccaccggtgataagcacaaaaaccgtaaaagcctgtttatgagcctgatgaccgacaacaacctggagtacctgcgtaaactgacccgtaactattggttcgaagataccgcgcgtctgcaggcgatggaccaaaccaacgtgtaccaggaagcgaccctggttctgctgaagattggcttccgttttgcggacgttccgttcgataccccggagcgtatggaacaatatgcgaaagacatgaacgcgatggtggatagctttggcagcatcggtaccgcgtacaccggttatcgtcgtgcgctgaacgcgcgtgaccgtgttgaggattacctggaaaagcagatcattgcgacccgtaagggcaaactgttcccggaggaaggcagcggtctgtatgagtttagccactggaaggacatgaacggtaaacacatggatagccgtctggcggcggtggacctgatgaacaccatccgtccgctggttgcgattaaccgtttcatcagctttggcgtgctggcgatgcacgagtacccgggtgaacgtgcgcgtgtggcgcagaacgacaacagctacgcgtataaattcgcgcaagaagtgcgtcgttactatccgttcgttccgtttctgccgggcaaggcgaaacaaaacattaccttcgagggttacaagatcgacaaagataccatgctgattctggatatctttggtaccctgcacagcgaaagcctgtgggagaacgcggaacagttctatccggagcgttttgaaaactgggacggcagcccgtttgatctgattccgcaaggtggcggtgattactataccaaccaccgttgcgcgggcgagtggatgaccatcattatcatggaggaaaccatgaagtacttcgcgcgtgaaatccagtatgacgtgccgccgcaagactttaccgttgatcgtaccaaactgccgggccagattaacagcggtatgattatcgagaacatccgtaacaacttcgatcgtcgtcgttaactcgag

SEQ ID NO: 160 - DNA, 1320 bp, Artificial Sequence
  Artificial codon optimized gene sequence
catatgcatcaccaccaccaccacgagaacctgtacttccagggcatggcgaagaaactgccgaaagataccggtctggacaacaccctgaagatcattaacgaagcgtacacctatgtgccgaagcgtctggagaaattcggcaccaaggcgtttgaaacccgtgcgctgggcatgaaaccggtggttgtgatcagcggcaaggcggcggcggagctgttttatgacaacgataaaatcagccgtaagggcaccctgccgaaacgtattgttcacaccctgttcggcaagggtgcgatccacaccaccgagggtaaagttcacgtggatcgtaaggcgctgtttatgagcctgatgaccgagaagaacctgaaatacctgcgtgaactgacccgtaactattggttcatgcacaccgagcgtatgcagaacaaagatgaggttaacgtgtaccaagaagcgggcctgattctgaccaaagtgggctttcgttgggcgggtctgaagcagaccgacgagcaggcggcgcaaaacgcggaagatatgaacaccatgatcgacagcttcagcggcctgggtcagagcctgaaaggttatcgtgaagcgaagaaagcgcgtgcgcgtgtggagcaatttctgcaggaccaaatcgaagcggtgcgtgcgggtcagcaatatgcggagccgggtaccgcgctgtatgaattcgcgcactggaaggacctgaacgatcaaccgatggatccgcacctgtgcgcggttgacctgatgaacattgtgcgtccgctggttgcggtgaaccgtttcgttagctacggtgtgaaagcgctgatcgagtttgatcaggagcgtaagaaactgcaagttacccacgacccgaactacgcgtataagttcgcgcaggaagtgcgtcgtattttcccgtttgtgccgtttctgccgggccgtctgaaacaaaccgtggaattcgatggctttaagctgaagaaaggtaccctgaccgttctggatatcttcggtaccacccacgacccggagctgtttgaaaacccgtatcagttcaacccggaccgttttgataactgggacggcagcccgttcgatctgattccgcaaggtggcggtgacttttacaccaaccaccgttgcgcgggcgagtggatgaccgttatcgtgatggaggaaaccattcagtatttcgcgaacaaaatcgactttgaagcgccggcgcaggatctgagcgttaaactggaccaattcccgggcaaggtgaccagcggtaccatcattaagagcgtttacccgcgtatttaactcgag

SEQ ID NO: 161 - DNA, 1317 bp, Artificial Sequence
  Artificial codon optimized gene sequence
catatgcatcaccaccaccaccacgagaacctgtacttccagggcatgaacaagattccgaaggacaaaggtctggataacaccctgaagatcgtgaaagaagcgtacacctatgttccgcgtcgtctggaggcgttcgacaccaacgtgtttgaaacccgtgcgctgggcatgaagaaagttctggtggttagcggtaagaaagcggcggagctgttttatgataacgaaaagatcagccgtaaaggcgcgctgccgaaacgtattgtgaacaccctgtttggtcagggtgcgatccacaccaccgagggcaagcaacacgttgaccgtaaagcgctgtttatgagcctgatgaccaccaagaacctgaaatacctgcgtgaactgacccgtaactattggttcgcgcacacccagcgtatggaggacatggatgaagtgaacgtttacgaggaagcgaccctgctgctgaccaagattggctttcgttgggcgggtctgaaacagacccacgaggaagcggtgcaaaacgcgaaggacatggatctgctgatcgatagctttagcgcgctgggtcaaagcccggcgggttataagaaagcgaagaaagcgcgtgagcgtgtggaaaagtttctggagcaccagattaaagcggttcgtgagggcgaacaatacgcggcgccgaacaccgcgctgtatgaattcgcgcactggaaggacctgaacgataaaccgatggacctgcacctgtgcgcggtggatctgatgaacgtggtgcgtccgctggttgcggttaaccgtttcgtgagctacgcggttaaagcgctgatcgagtttgaccaggaacgtctgaagctgcaggttaccaaagatcaaaactacgcgtataagtttgcgcaggaagtgcgtcgtattttcccgtttgttccgttcctgccgggccgtctgaaacaaaacatcgaatttgacggttacagcctgaagaaaaacaccctgatcattctggacgtgttcggtaccacccacgatccgaagctgtttgagaacccgtatcagttcaacccgaaccgttttgaagactgggatggcagcccgttcgacctgatcccgcaaggtggcggtgatttttacaccaaccaccgttgcgcgggcgagtggatgaccgtgatcgttatggaggaaaccatcaactacttcgcgaacaagatcgacttcagcgtgccgacccaggacctgagcgttaagctggatcaattcccgggcaaagttaccagcggtaccgtgatccgtaacgttcgtccgcgtcgttaactcgag

SEQ ID NO: 162 - DNA, 1326 bp, Artificial Sequence
  Artificial codon optimized gene sequence
catatgcatcaccaccaccaccacgagaacctgtactttcagggcatgagcaagcaactgaacatcccgaaagaggaaggcctggaccacagcctggcgctgatccaggaaggttaccaatatattaccagccgtcgtaccaagtttaaaagcgacctgttcgaaacccgtctgctgggccagaaagtggtttgcattggtggccaagagggtgcggaactgttttatgacaacgagaagttcgaacgtaagggtgcggttccgaagcgtatccagaaaagcctgtttggcgaaaacgcgattcacaccctggatggcgaggcgcacgaacaccgtaagctgctgttcatgagcctgatgaccgaggaacgtctggagctgctggcggaactgacccgtgaggaatggcgtctggcggcggcggagtgggcgaaaatggaacaggtggttctgtttgacgaggttcagcaaattctgtgccgtgttgcgtgccgttgggcgggcgttccgatcgatgcgattgaagtgaagccgctggcgcgtgactttattgcgatggttgatgcgtttggtgcggtgggtccgcgtcaccaaaagggccgtaaagcgcgtaagaaaatggaggactacttcaagggtctgatcattgaaatccgtcaccgtcgtatcgagccgattgaaggtaccgcgctgcacaaaattgcgtggcaccgtgaccacaaggagaaacacctggatgaacacaccgcggcggtggagctgctgaacgttctgcgtccgatcgtggcgattacctacctggttacctttggcgcgctggcgctgttcgagcacaacgtgtggaaggaaaaactgcagagcggtgatgaggaaagcgttcagatgttcgcgcaagaagtgcgtcgtttctatccgtttgcgccgttcctgggtgcgaaggttaaaaacggctttgagtggaagggtcacaccttcgaaaaaggccaactggtgctgctggacgtttacggtaccaaccacgataaccgtctgtgggaggaaccgtatgtgtttaacccggagcgtttccgtaactggaacggtggcatgtttgacctgatcccgcagggtggcggtgatgagtacaccggccaccgttgcccgggtgaaatggcgaccatccaagtgatgaaaaccagcttcagctttctgaccaacgagatgatttataacgtgccggttggtcaggacctgagctacagcctgagccgtatgccgacctatccggaaagcggcttcatcattcagggtgttagcatcaagagccaaaccaccgtgtaactcgag

SEQ ID NO: 163 - DNA, 1326 bp, Artificial Sequence; identical to SEQ ID NO: 162
  Artificial codon optimized gene sequence

SEQ ID NO: 164 - DNA, 1329 bp, Artificial Sequence
  Artificial codon optimized gene sequence
catatgcatcaccaccaccaccacgagaacctgtacttccagggcatgggtaaaagcattccgcgtgaccgtggcatggataacaccgttaaggcgctgaagaaaggttacacctatattccggagctgcgtgaacgtctgaacagcaacgatatcgttgcgacccgtgcgctgggtggcaagaaaaccgtgatcattagcggcaaagacgcggcggagctgttttacgacaacgataagatcgaacgtcaaggtaccctgccgccgcgtgtggttaagaccctgttcggcaaaggtgcgattcacaccaccagcggcaagacccacatcgatcgtaaagcgctgtttatgagcctgatgaccgaggaaaacctgagctatctgcgtgagctgacccgtctggaatggtactataacaccacccgtatgagccacatggacgaggtgaacgtttacaaagaagcgatttatgtgctgaccaaggttggcatcaaatgggcgggtctgcacgcgagcgaggatgaaattaaccagtacgcggaggacatggataccatgatcgacagcttcaaagcgattggcaccatgtttaagggttataaagaagcgcgtaaggcgcgtgatcgtgttgaaacctggctggaagcgcagattcaaggcgtgcgtgatggtgaaattaccccgccggaaggtaccgcgctgtacgagttcgcgcactggaaagactatgaaggtaacccgatggatgcgcgtctggcggcgatcgacctgatgaacgtgattcgtccgctggttgcgatcaaccgtttcgttgcgtttggtaccctggcgatgtacgagttcccgaaggaacgtaccaaagtggcgcagaacgaggaagattacgtgtataagtttatccaagaggttcgtcgtttctacccgtttgtgccgtatctgccgggcaaggcgggtaaagacattgagttccgtggctacgaaatcaagaaagacacctttatggtgctggatgtttatggtaccctgcaccgtgaggacctgtgggaaaacgcggatcagttcattccggagcgttttgaagactgggatggcagcccgttcgatctgatcccgcaaggtggcggtgactactataccaaccaccgttgcgcgggcgagtggatgaccgtgattgttatggaggaaagcatgaaatactttgcgcaggcgatcaagtatgaagttccggcgcaagacctgaccgtggatctgaacaagctgccgggccgtgttaaaagcggtttcatcattaagaacgtggaacgtgacatcaaccgtttttaactcgag

SEQ ID NO: 165 - DNA, 1320 bp, Artificial Sequence
  Artificial codon optimized gene sequence
catatgcatcaccaccaccaccacgaaaacctgtactttcagggcatggcgaagattaaacgtgacaagggtctggataacaccctgaaggttttcaaacagggctacctgtataccaccaaccaacgtgagcgtctgggtgcggaagtgtttgaaacccgtgcgctgggtggcaaaagctacgtggttctgagcggcaaggaaggtgctgagctgttctatgacaacgataaaatcgaacgtgagggtggcctgccgaagcgtgtggttaacaccctgttcggcaaaggtgcgattcacaccaccaccggcaaacaacacatcgatcgtaaggcgctgtttatgagcctgatgaccgaaggtaacctggagtacgttcgtaaactgacccgtaactattggaaggcgaacaccgcgcgtatggaagcgatgggcgacgtgaacgtttaccacgagagcattgttctgctgacccgtattggtatgcgttgggcgggtgtgaccgcgccggaggaagagattgaacgtatcgcggaggacatggatattatgatcgatagctttaagggcctgggtaacgcgttcaagggttataaaagcagcaaggacgcgcgtaaacgtgtggaagactggctggaggatcagatcattaagacccgtaacggcgaaattaacccgccgaaaggtagcagcctgtacgaactggcgcactggcgtgactatgagggcaaccaaatggatagccgtctggcgggtatcgacctgatgaacacctttcgtccgctgattgcgatcaaccgtttcgtgagctacggtgttctggcgctgtatgaacacccggaggcgatccgtaaaatgagcaccgttgaagactacccgtatatgttcgcgcaggaagtgcgtcgtttctacccgtttgtgccgttcctgccgggcaagaccaaagtggaagttgagcacaaaggtgtgaccattccgaaggaccagcgtctggtgatcgatgtttatggtaccctgcacagcgaagagctgtggaaccaaccgaacaaatttattccggaacgtttcaaggactgggatggcagcccgttcgatatgatcccgcagggtggcggtgactactggaccaaccaccgttgcgcgggcgagtggattaccatcatcatcatggaagaaaccatgaagtacttcgcgaacgaaattacctgggatgttccggagcaagacctgaccatcgatctgaacagcattccgggctacatcaacagcggtatggtgatcaacaacgttcgtgagaaagtggaccgtttttaactcgag

SEQ ID NO: 166 - DNA, 1296 bp, Artificial Sequence
  Artificial codon optimized gene sequence
catatgcatcaccaccaccaccacgagaacctgtacttccagggcatgaacgtgaacgttgcgaaagaccgtctggatcaaaccatcagcctggttcgtgagggttacgcgtttattccgaaccgtctgcacagccagcacctggacgtgtatgaaaccaaactgctgggccaacgtatcgcgctgattggtggcgaggaaggtgcgaacctgttctatgatagcagccgtatgaagcgtaacggtgcgctgccgaaaccggttctgaagaccctgttcggcgagggtggcgtgcataccaaagatggtgcggcgcatgcgcaccgtaagcagctgtttatgagcctgatgaccccggatgcgctgcgtcgtctgagcaacctgaccaccgcgtactgggaggaatatgcgatgaagtgggaaaaacagcgtaaggtggttctgtacgacgagagccgtgaactgctgtgccaagtggcgtgcgattgggcgggtatcccgctgggtaagaaagaggtggaacaacgtaccgaggacctgagcaaaatgatcgatggcttcagcgcgctgggtccgaagcacattgaaagccgtcgtgcgcgtaaccgtagcgagaaatggattcgtagcatcattgaagacgttcgtgcgaacaagctggaaacccgtgagggcagcgcgatccacgagatggcgtttcacctggatgaaaacggtgaccgtctggataccgagattgcggcggtggaactgctgaacgttatccgtccgctggttgcgattagcaaattcctggcgtttggcgcgaaggcgtacgcggactatccggaaacccgtgagaagaccaaagagagcgatgaatacatcctgcgtttcgcgcaggaagttcgtcgttactatccgttcgcgccgtttctgggcgcgaaggtgaaacacgacttcacctggcagggttatacctttcgtgagaaccaactggttatgatcgacctgttcggtaccaaccacgatccgcgtctgtgggaaaacccggaccagttcgacccggatcgttttaaagactggaagggtggcctgtttgatctgattccgcaaggtggcggtgactactatgatggtcatcgttgcccgggcgagtggccgaccatcgaagttctgaaagcgagctttggctacctgagccgtaagattgactataaagtgccgaagcaagatcacagctacagcctgagcaaaatgccggcgctgccgaagagcggtatgatcctgaagaaagtgcgtcgtaagtaactcgag

SEQ ID NO: 167 - DNA, 1314 bp, Artificial Sequence
  Artificial codon optimized gene sequence
catatgcatcaccaccaccaccacgagaacctgtactttcagggcatgagcaaactgaaggaaagcggtttcgatcacaccctgaccatgctgaaagagggctacacctatattccgaaccgtaccgaaaagtaccaaagcccgatcttctttaaccgtgttctgggtggcaaaccgtgcgcgatcattagcggcaaagaggcggcggaactgttttataacaacgacctgattgagcgtaaaggtaccatgccgaaacgtatcgttaagaccctgtttggcaagggtgcgattcacaccaccaccggtaaagtgcacatcgatcgtaaggcgctgttcatgagcctgatgaccgacgaaaacctgagcctgctgcgtcagattaccaaagacgcgtgggagaacgaagttgatagcctgatcaaggtgaacgaggtgaacgtttacaagaaaagcagcgaagtgctgaccaaagttggcctgaagtgggcgggtattgaggacaccccgaaaaacattgataaaatcgcgaaggaaatggacctgatgatcgatagcttcggccagctgggtggcgcgtacaaaggttatcgtgcggcgcaaaaggcgcgtgcgaacgttgaggcgtacctgcagaaccaaattaagaaagtgcgtaaaggcaagctgaaaccgcgtgaaaacagcgcgctgtatgcgtttagccactggaaagacatgaacaacaagccgatggatctggcgctgtgcgcggtggacctgatgaacgttattcgtccgctggttgcgatcaaccgtttcgtgacctacggtgttctggcgatccacgagttcaacggcgaaaaggataaactgaagtttggtaccgacgattacgtttatcagttcgtgcaagaggttcgtcgtttctacccgtttgtgccgtatctgccgggcaaagcgaagaaaaagctgacctttgacaaccagaagattaaaaaggaccaaatgatcctgctggatgtgtacggtaccctgcacaacgagaaactgtgggaatatccgaaccgtttcgatccgacccgttttgaaaactgggacggcagcccgttcgatctgatcccgcagggtggcggtgactatcacaccaaccaccgttgcgcgggcgagtggatgaccatcattatcatggaggaaaccatgaagttctttacccgtaccattagcttcgaagttccgccgcaagactttaccgtggatctgaccaaactgccgggcaaggttgcgagcggtatgaacatccgtcaggtgcaagcgctgaacaactaactcgag

SEQ ID NO: 168 - DNA, 1308 bp, Artificial Sequence
  Artificial codon optimized gene sequence
catatgcatcaccaccaccaccacgctaaaaaattaccaaaagtaaaggggctggacaacaccgttgatatcattaaaggtggctacacctatgtgccgggcaagctggaggagttcgacagcaaagcgtttgaagtgcgtgcgctgggtggcaagaaaatcgcggttatgagcggcaaagaggcggcggaaattttctacgataacgagaagatggaacgtcagggcaccctgccgaaacgtatcgtgaacaccctgttcggcaagggtgcgattcacaccaccgcgggcaagaaacacgttgaccgtaaagcgctgtttatgagcctgatgaccgatgagaacctgaactacctgcgtgaactgacccgtaactattggttcatgaacaccgagcgtatgcaaagcatggacaaagtgaacgtttacaacgaaagcatctatatgctgaccaagattggctttcgttgggcgggtatcattcagaccccggaggaagcggagcaaaacgcgaaagacatggataccatgatcaacagcttcgtgagcctgggcagcgcgtacaagggttataagaaagcgaagaaagcgcgtaagcgtgtggaggactttctggaaaaacagatcattgatgttcgtaagggcaaactgcacccggaggaaggcaccgcgctgtacgagttcgcgcactgggaagacctgaacgataacccgatggacagccacctgtgcgcggttgatctgatgaacgtggttcgtccgctggcggcgatcaaccgtttcattagctatggtgtgaaagttctgattgaatttgaccaggagaaggaaaaactgcgtctggagaacaacgaagattacgcgtataagtttgcgcaagaggtgcgtcgtatcttcccgtttgttccgtacctgccgggtcgtgcggcggtggacctggaatacgatggctataaaattccggcgggtatgatgaccgcgctggacgtttatggcaccacccacgacgaggatctgtgggaaaacccggatcagttcaacccgaaccgttttgacaactgggatggcagcccgttcgacctgatcccgcaaggtggcggtgatttttacaccaaccaccgttgcgcgggcgagtggatcaccgtgatcattatggaggaaaccatgaagtacttcgcgaacaagatcgaatttgacgtgccgagccaagatctgagcgttaagctggacaaactgccgggcaacgttaccagcggcaccatcattagcaacgtgcgtccgcgtgttgcgcgtaagtaatgaaagctt

SEQ ID NO: 169 - DNA, 1329 bp, Artificial Sequence
  Artificial codon optimized gene sequence
catatgcatcaccaccaccaccacgaaaacctgtacttccaaggcatggcgaagcgtatgccgaaagaccgtggtctggataacaccctgaccattatgaaagagggttacgaatatatccagaaccgtaccaagaaattcaacagcaacgtgtttgaaacccgtgtgctgggtggcaagcaagcggtggttattagcggcaaacaggcggcggaactgttttatgacaacgataaaattgagcgtagcggtaccctgccgccgcgtgtggttaagaccctgttcggcaaaggtgcgattcacaccaccaccggtaagaaacacatcgatcgtaaggcgctgtttatgagcctgatgaccgacgaaaacctgacctacctgcgtaaactgacccgtaactattggttccagaacacccaacgtatgcagctgatggatgaagtgaacgtttacaaggagagcatcattctgctgaccaaaattggctttcgttgggcgggtatcattgaaaccccggaaaacatcgagcaacacgcgatggacatggataagatgattgatagcttcggcgcgatcggtagcccgtacaaaggctatcgtgaagcgaagaaagcgcgtacccgtgttgaggactttctggaggaacaaatcattaagacccgtaagggcaaaattcatccggagccgggtagcgcgctgtacgaattcagccactgggaggacatgaacggtcagccgatggatagccgtctgtgcagcgtggacctgatgaacgttattcgtccgctggttgcgatcaaccgtttcgtggcgtatggcgttctggcgatgcacgaatttccgggcgagcgtacccgtgttgcgcgtgacgatgacgattacgcgtataagttcgtgcaagaagttcgtcgttactatccgttcgtgccgtttctgccgggcaaggcggcgaagaacatcgagttcgatggttacaagatcaagaaagacaccatgatcattctggatgtgttcggtaccctgcaccgtgaagacacctttagcgagccggaacgtttcaacccgtaccgttttgacaactgggatggcagcccgttcgatctgatcccgcagggtggcggtgactatcacaccaaccaccgttgcgcgggtgaatggatgaccatcattatcatggaggaaaccatgaagtacttcgcgcaagagattgactatgatgttccgccgcaggactttaccgtggatcgtaccaagctgccgggccgtgttaaaagcggtatgattatcaacaacgtgcgtctgaacatcgaccgtaccaaataactcgag

SEQ ID NO: 170 - DNA, 1329 bp, Artificial Sequence
  Artificial codon optimized gene sequence
catatgcatcaccaccaccaccacgaaaacctgtacttccagggcatgagcaagccgattccggtggacaagggtatcgataaaaccctgaccgttatgaaagagggttacgaatatgtgagcaaccgtaccaagaaattcaacagcgatatttttgaaacccgtgttctgggtggcaagcgtgcgatggtgatcagcggcaaagaggcggcggaactgttctatgacaacgataagatcgagcgtaaaggtaccctgccgccgcgtgtggttaagaccctgtttggcaaaggtgcgattcacaccaccaccggcaaggttcacatcgaccgtaaaagcctgttcatgagcctgatgaccgaaaacaacctggagtacctgcgtaagctgacccgtaacttttggtatcagaacaccgaacgtatgcaactgatgggcaaggtgaacgtttacaaagagagcatcattctgctgaccaaagttggcttccgttgggcgggtgtgaccgagaagccggaaaaaattgagcagcacgcgatggacatggataagatgatcgatagcttcggcagcgtgggtaccgcgtacaaaggctatcgtgaagcgaagaaagcgcgtgcgcgtgttgaagactttctggagaagcagatcattgcgacccgtaagggcaaaattcacccggaggaaggtaccgcgctgcacagcttcagccactggcgtgactttcagggtaactacatggatagccgtctggcggcggtggacctgatgaacgttattcgtccgctggttgcgatcaaccgtttcgtggcgtatggcgttctggcgctgcacgagtttccgggcgagcgtgaacgtgtgagcgcggatgaaaacaactacgcgtataaatttacccaagagctgcgtcgttacttcccgtttgttccgttcctgccggcgaaggcgaaagttgacattgattttcagggccacgtgatcccgcaagacaccatgattgttctggatatcttcggtaccctgcaccgtgaagacctgtgggaagatccggagcgtttctacccggatcgttttgagaactgggacggcagcccgtttgatctgatcccgcagggtggcggtgactatcacaccaaccaccgttgcgcgggtgaatggatgaccatcattatcatggaggaaaccatgaagtacttcgcgcgtgagattacctatgacgttccggcgcaagactttaccgtggatcgtaccaagctgccgggccgtgtgaaaagcggtatggatatcgagaacgttcgtctgaacttcgaccgtaccctgtaactcgag

SEQ ID NO: 171 - DNA, 1323 bp, Artificial Sequence
  Artificial codon optimized gene sequence
catatgcatcaccaccaccaccacgagaacctgtactttcagggcatgagcaccatcaagcgtgacaaaggtctggataacaccctgaaggtgatgaaagagggctacctgtataccaccaaccaacgtaaccgtctgggcgcggataacatctttgaaacccgtggtctgggtggcaagcgtgtgctggttctgagcggcaagaaagcggcggaaaccttctatgacaacgataagattgaacgtaaaggtaccctgccgaagcgtgtggttaacaccctgtttggcaaaggtgcgatccacaccaccaccggcaagaaacacattgatcgtaaggcgctgttcatgagcctgatgaccgagggtaacctgaaacacctgcgtgaactgacccgtaaccactggtacatgaacacccaccgtatggagcagatggaccaaatcaacatttatcgtgaaagcatcattctgctgaccaaagtgggcaccaaatgggcgggtgttcaggcgccggaggaaaaaatcgaggaaattgcgaccgacatggatatcatgattgacagctttcgtggcctgggtaccgcgttcaagggttaccgtagcagcgtgaaagcgcgtcgtcgtgttgaggactggctggaagatcagatcattcaaacccgtaagggcaaaatctttccgccggaaggtaccgcgctgtacgagttcgcgcactgggaagactatctgggcaacccgatggatagccgtctgtgcgcgattgacctgatgaacaccttccgtccgctgatcgcgattaaccgtttcgtgagctttggtctgctggcgatgcacgagtttccggttagccgtgagaagatcaacaacgaagacgattacgcgtatatgttcagccaagaagtgcgtcgtttctacccgtttgttccgttcctgccggcgaaggcgaaaaccgacttccactttgatggccacaaggtggacaaagataccatgctgctgctggatatctatggtaccatgcaccgtgaggacgtttttgaaaacccgaacgagtttcgtccggaacgtttcctggactgggatggtagcccgttcgatctgattccgcagggtggcggtgactactataccaaccaccgttgcgcgggcgagtggatgaccgtgatcattatggaggaaaccatgaaatactttgcgggtcgtatcacctatgatgtgccggaacaagacctgcgtgttgatctgaacagcatcccgggctacgttaagagcggtttcgtgattgagaacgttaaagaaaacctggaccgtcgttaactcgag

SEQ ID NO: 172 - DNA, 1323 bp, Artificial Sequence
  Artificial codon optimized gene sequence
catatgcatcaccaccaccaccacgaaaacctgtacttccaaggcatgagcaccattaagaaagacaagggtctggataacaccctgaaggcgatcaaacagggctacaagtataccgaaaaccaacgtaaacgtctggacagcaacaacatttttgaaacccgtggtctgggtggtaaacgtatcattttcatcagcggtaaagaggcggcggaaaccttttatgacaacgataagatcgagcgtgagggtatgctgccgaaacgtgtggttagcaccctgttcggcaagggtgcgattcacaccaccaccggcaaggtgcacatcgatcgtaaagcgctgtttatgagcctgatgaccgagggtaacctgcagtacctgcgtgaactgacccgtaaccaatggcgtagcaacaccattaacatggaacgtcaggacaccgtgaacgtttatcgtgaggcgattgtgcaactgacccacatcggcacccgttgggcgggtgttcaggcgccggcggaggaaattgatcgtatcgcgaccgacatggatctgatgatcgacagcttcaaaggcctgggtctgagcttcaagggttttcgtgaaagcaaagcggcgcgtcgtcgtgtggaggactggctggaagatcagattcacaagacccgtaaaggcgttattcacccgccggagggtaccgcgctgtacgaattcgcgcactgggaggactttgaaggcaacccgatggatagccgtctgtgcgcgattgacctgatgaacaccttccgtccgctgattgcgatcaaccgtttcgtgacctttggtgttctggcgctgcaccaatatccggaaacccgtgagaagatcaaaaacgacccggattacgcgtatatgtttagccaggaagtgcgtcgttactatccgttcgttccgtttctgccggcgaaggcgaaagtggatttcgagtttgaaggccacaagattgacaaagatgtgatgatcctgctggatgtttacggtaccatgcaccgtgacgatgttttcgacaacccgaacgagttctatccggaacgttttaaggactgggatggcagcccgtttgatctgatcccgcaaggtggcggtgactaccacaccaaccaccgttgcgcgggtgaatggatgaccatcattatcatggaggaaaccatgaaatacttcgcggagcgtattacctatgacgtgccggaacaggacctgaccgttgatctggagaagctgccgggcagcgttgcgagcggtatgaacatcaccaacgtgcgtgagaacgttaaccgtaaataactcgag

SEQ ID NO: 173 - DNA, 1326 bp, Artificial Sequence
  Artificial codon optimized gene sequence
catatgcatcaccaccaccaccacgagaacctgtacttccagggcatggcgaccatcaagcgtgataaaggtctggacaacagcgtgaaggttatgaaacagggctacctgtataccaccaaccaacgtgagcgtctgggcgtgaccgacggtgttttcgaaacccgtgcgctgggtggcaagcgtatcattgttctgagcggcaaagatggtgcggagctgttttatgataacgacaagattgaacgtagcggtaccctgccgaaacgtgtggttaacaccctgttcggcaagggtgcgatccacaccaccaccggcaaggtgcacattgaccgtaaagcgctgtttatgagcctgatgaccgagggtaacctgaaatacctgcgtgaactgacccgtaacctgtggtttgcgaacacccagcgtatggagagcatggatcaagtgaacgtttatcgtgaaagcatcattgtgctgaccaaagttggtacccgttgggcgggtgttcaagcgccggagaaggaaatcgagaacattgcgaccgatatggacatcatgattgacagcttcaaagcgatcggcagcgcgtttaagggttaccgtgaggcgaaagcggcgcgtcgtcgtgtggaagattggctggaggaccagatcattcaaacccgtaagggcaaaatccacccgccgaagggtaccgcgctgtacgagttcgcgcactggaaggattataaaggcgaaccgatggacagccgtctgtgcggtattgatctgatgaacacctttcgtccgctgatcgcgattaaccgtttcgttgcgtttggtgcgctggcgatgtacgagaacccggtggcgcgtgaaaagatcaaacaggacgatgactacgcgtatatgttcgcgcaagaagttcgtcgtttctacccgtttgtgccgtatctgccggcgaaggcgaaagttgactttcagtacaagggctatgaaatcgagaaagataccatgctggcgctggacatttacggtaccatgcacgatccgaacgtgtgggaagacccgaacgagttctatccggaacgttttaaggattgggatggcagcccgtttgacctgatcccgcaaggtggcggtgattaccacaccaaccaccgttgcgcgggcgagtggatgaccgttatcattatggaggaaaccatgaaatacttcgcgagccgtattaactatgacgttccggaacaggatctgaccgtggacctgaacagcctgccgggctatatcaagagcggttttgtgattgagaacgttcaagaaaacgtggatcgtacctaactcgag

(19) United States
(12) Patent Application Publication
(10) Pub. No.: US 2021/0139879 A1
(43) Pub. Date: May 13, 2021

(54) Consumer Product Compositions Comprising P450 Fatty Acid Decarboxylases

(71) Applicant: The Procter & Gamble Company, Cincinnati, OH (US)

(72) Inventors: Jean-Luc Philippe Bettiol, Etterbeek (BE); Denis Alfred Gonzales, Brussels (BE); Juan Esteban Velasquez, Cincinnati, OH (US); Hilda Andamiche Namanja-Magliano, Loveland, OH (US)

(21) Appl. No.: 17/083,345

(22) Filed: Oct. 29, 2020

Related U.S. Application Data
(60) Provisional application No. 62/928,580, filed on Oct. 31, 2019.

(51) Int. Cl. C12N 9/88; C11D 3/386

(52) U.S. Cl. CPC C12N 9/88; C11D 3/38636; C12Y 401/01

(57) ABSTRACT

Consumer product compositions having P450 fatty acid decarboxylases and methods of using said consumer products to provide a benefit by converting long chain fatty acids present in soils into terminal olefins.

### REFERENCE TO A SEQUENCE LISTING

This application contains a Sequence Listing in computer readable form. The computer readable form is incorporated herein by reference.

### FIELD OF THE INVENTION

The present invention relates to consumer product compositions comprising P450 fatty acid decarboxylases and methods of using said consumer products to provide a benefit by converting long chain fatty acids present in soils into terminal olefins.

### BACKGROUND OF THE INVENTION

Consumer product compositions, such as those for cleaning surfaces, may need to have a good suds profile, in particular a long-lasting suds profile especially in the presence of greasy soils, while providing good soil and/or grease cleaning. Indeed, consumers frequently see suds as an indicator of the performance of consumer product compositions, such as detergent compositions. Moreover, the user of a detergent composition may also use the suds profile and the appearance of the suds (e.g., density, whiteness) as an indicator that the wash solution still contains active detergent ingredients. Accordingly, it is desirable for a detergent composition to provide “good sudsing profile”, which includes good suds height and/or density as well as good suds duration during the initial mixing of the detergent with water and/or during the entire washing operation.

It has been found that some types of soil, in particular greasy soils comprising long chain fatty acids, such as stearic acid, oleic acid, linoleic acid, and linolenic acid, can act as a suds suppressors, triggering consumers to replace the product more frequently than is necessary. As such there is a need to provide consumer product compositions with desirable suds properties, especially in the presence of greasy soils, even more in the presence of greasy soils comprising long chain fatty acids, and that at the same time provide good soil and grease removal.

The use of two different classes fatty acid decarboxylases, OleT-like and UndA-like, to enhance the sudsing profile of detergent compositions have been previous reported (EP 3,243,896B1). However, these enzymes usually have a strong preference for medium chain length fatty acids (e.g. C12, C14), while longer fatty acids (e.g. oleic acid) are converted slowly or not converted. Thus, there is still a need for fatty acid decarboxylases that transform long chain fatty acids efficiently.

There is also a desire to utilize less surfactant materials in consumer product composition. However, using less surfactant can decrease the suds generation and/or cleaning performance of the consumer product composition.

There remains a desire to provide a consumer product composition for cleaning surfaces that have soils comprising long chain fatty acids, which provides effective suds generation and/or cleaning performance, especially when the consumer product composition contains relatively low amounts of surfactant.

### SUMMARY OF THE INVENTION

The present invention relates to consumer product compositions comprising P450 fatty acid decarboxylases and methods of using said consumer products to provide a benefit by converting long chain fatty acids present in soils into terminal olefins.

The present invention provides a consumer product composition comprising a P450 fatty acid decarboxylase; wherein said decarboxylase comprises a polypeptide sequence having at least about 80% identity to one or more sequences selected from the group consisting of: SEQ ID NO: 2, 22, 44, 60, 65, 71, 83, 117, 121, 122, 156, and their functional fragments thereof; preferably SEQ ID NO: 2, 60, 65, 71, 83, 122, and their functional fragments.

The present invention also provides a detergent composition with desirable suds properties, even in the presence of greasy soils comprising long chain fatty acids, while at the same time providing good soil and grease removal. The detergent composition is particularly suited for manually washing soiled articles, preferably dishware. When the composition of the invention is used according to this method a good sudsing profile, with a long lasting effect is achieved.

The elements of the composition of the invention described in relation to the first aspect of the invention apply mutatis mutandis to the other aspects of the invention.

### BRIEF DESCRIPTION OF THE DRAWINGS

The FIGURE is a drawing showing SSN of OleT Decarboxylases.

### DETAILED DESCRIPTION OF THE INVENTION

Definitions

As used herein, the articles “a” and “an” when used in a claim, are understood to mean one or more of what is claimed or described.

As used herein, the term “substantially free of” or “substantially free from” means that the indicated material is present in an amount of no more than about 5 wt %, preferably no more than about 2%, and more preferably no more than about 1 wt % by weight of the composition.

As used therein, the term “essentially free of” or “essentially free from” means that the indicated material is present in an amount of no more than about 0.1 wt % by weight of the composition, or preferably not present at an analytically detectible level in such composition. It may include compositions in which the indicated material is present only as an impurity of one or more of the materials deliberately added to such compositions.

By “consumer product composition”, as used herein, it is meant compositions for treating hair (human, dog, and/or cat), including bleaching, coloring, dyeing, conditioning, growing, removing, retarding growth, shampooing, and styling; personal cleansing; color cosmetics; products relating to treating skin (human, dog, and/or cat), including creams, lotions, ointments, and other topically applied products for consumer use; products relating to orally administered materials for enhancing the appearance of hair, skin, and/or nails (human, dog, and/or cat); shaving; body sprays; fine fragrances such as colognes and perfumes; compositions for treating fabrics, hard surfaces and any other surfaces in the area of fabric and home care, including air care, car care, dishwashing, fabric conditioning (including softening), fabric freshening, laundry detergents, laundry and rinse additive and/or care, hard surface cleaning and/or treatment, and other cleaning for consumer or institutional use; products relating to disposable absorbent and/or non-absorbent articles including adult incontinence garments, bibs, diapers, training pants, infant and toddler care wipes; hand soaps; products relating to oral care compositions including toothpastes, tooth gels, mouth rinses, denture adhesives, and tooth whitening; personal health care medications; products relating to grooming including shave care compositions and composition for coating, or incorporation into, razors or other shaving devices; and compositions for coating, or incorporation into, wet or dry bath tissue, facial tissue, disposable handkerchiefs, disposable towels and/or wipes, incontinence pads, panty liners, sanitary napkins, and tampons and tampon applicators; and combinations thereof.

As used herein, the term “detergent composition” refers to a composition or formulation designed for cleaning soiled surfaces. Such compositions include but are not limited to, dishwashing compositions, laundry detergent compositions, fabric softening compositions, fabric enhancing compositions, fabric freshening compositions, laundry pre-wash, laundry pretreat, laundry additives, spray products, dry cleaning agent or composition, laundry rinse additive, wash additive, post-rinse fabric treatment, ironing aid, hard surface cleaning compositions, unit dose formulation, delayed delivery formulation, detergent contained on or in a porous substrate or nonwoven sheet, and other suitable forms that may be apparent to one skilled in the art in view of the teachings herein. Such compositions may be used as a pre-cleaning treatment, a post-cleaning treatment, or may be added during the rinse or wash cycle of the cleaning process. The detergent compositions may have a form selected from liquid, powder, single-phase or multi-phase unit dose or pouch form, tablet, gel, paste, bar, or flake. Preferably the composition is for manual-washing. Preferably, the detergent composition of the present invention is a dishwashing detergent. Preferably the composition is in the form of a liquid.

As used herein, the term “soiled surfaces” refers non-specifically to any type of flexible material consisting of a network of natural or artificial fibers, including natural, artificial, and synthetic fibers, such as, but not limited to, cotton, linen, wool, polyester, nylon, silk, acrylic, and the like, as well as various blends and combinations. Soiled surfaces may further refer to any type of hard surface, including natural, artificial, or synthetic surfaces, such as, but not limited to, tile, granite, grout, glass, composite, vinyl, hardwood, metal, cooking surfaces, plastic, and the like, as well as blends and combinations, as well as dishware. Key targeted soiled surfaces by this application are soiled dishware.

As used herein, the term “water hardness” or “hardness” means uncomplexed cation ions (i.e., Ca²⁺ or Mg²⁺) present in water that have the potential to precipitate with anionic surfactants or any other anionically charged detergent actives under alkaline conditions, and thereby diminishing the surfactancy and cleaning capacity of surfactants. Further, the terms “high water hardness” and “elevated water hardness” can be used interchangeably and are relative terms for the purposes of the present invention, and are intended to include, but not limited to, a hardness level containing at least about 12 grams of calcium ion per gallon water (gpg, “American grain hardness” units).

As used herein, the terms “protein,” “polypeptide,” and “peptide” are used interchangeably herein to denote a polymer of at least two amino acids covalently linked by an amide bond, regardless of length or post-translational modification (e.g., glycosylation, phosphorylation, lipidation, myristilation, ubiquitination, etc.). Included within this definition are D- and L-amino acids, and mixtures of D- and L-amino acids.

As used herein, “polynucleotide” and “nucleic acid” refer to two or more nucleosides that are covalently linked together. The polynucleotide may be wholly comprised ribonucleosides (i.e., an RNA), wholly comprised of 2′ deoxyribonucleotides (i.e., a DNA) or mixtures of ribo- and 2′ deoxyribonucleosides. While the nucleosides will typically be linked together via standard phosphodiester linkages, the polynucleotides may include one or more non-standard linkages. The polynucleotide may be single-stranded or double-stranded, or may include both single-stranded regions and double-stranded regions. Moreover, while a polynucleotide will typically be composed of the naturally occurring encoding nucleobases (i.e., adenine, guanine, uracil, thymine, and cytosine), it may include one or more modified and/or synthetic nucleobases (e.g., inosine, xanthine, hypoxanthine, etc.). In embodiments of the invention, such modified or synthetic nucleobases will be encoding nucleobases.

As used herein, “coding sequence” refers to that portion of a nucleic acid (e.g., a gene) that encodes an amino acid sequence of a protein.

As used herein, “naturally occurring,” “wild-type,” and “WT” refer to the form found in nature. For example, a naturally occurring or wild-type polypeptide or polynucleotide sequence is a sequence present in an organism that can be isolated from a source in nature and which has not been intentionally modified by human manipulation.

As used herein, “non-naturally occurring” or “engineered” or “recombinant” when used in the present invention with reference to (e.g., a cell, nucleic acid, or polypeptide), refers to a material, or a material corresponding to the natural or native form of the material, that has been modified in a manner that would not otherwise exist in nature, or is identical thereto but produced or derived from synthetic materials and/or by manipulation using recombinant techniques. Non-limiting examples include, among others, recombinant cells expressing genes that are not found within the native (non-recombinant) form of the cell or express native genes that are otherwise expressed at a different level.

As used herein the term “identity” means the identity between two or more sequences and is expressed in terms of the identity or similarity between the sequences as calculated over the entire length of a sequence aligned against the entire length of the reference sequence. Sequence identity can be measured in terms of percentage identity; the higher the percentage, the more identical the sequences are. The percentage identity is calculated over the length of comparison. For example, the identity is typically calculated over the entire length of a sequence aligned against the entire length of the reference sequence. Methods of alignment of sequences for comparison are well known in the art and identity can be calculated by many known methods. Various programs and alignment algorithms are described in the art. It should be noted that the terms ‘sequence identity’ and ‘sequence similarity’ can be used interchangeably.

As used herein, “percentage of sequence identity,” “percent identity,” and “percent identical” refer to comparisons between polynucleotide sequences or polypeptide sequences, and are determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage is calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity.

As used herein, the term “variant” of P450 fatty acid decarboxylase enzyme means a modified P450 fatty acid decarboxylase enzyme amino acid sequence by or at one or more amino acids (for example 1, 2, 3, 4, 5, 6, 7, 8, 9 or 10 or more amino acid modifications) selected from substitutions, insertions, deletions and combinations thereof. The variant may have “conservative” substitutions, wherein a substituted amino acid has similar structural or chemical properties to the amino acid that replaces it, for example, replacement of leucine with isoleucine. A variant may have “non-conservative” changes, for example, replacement of a glycine with a tryptophan. Variants may also include sequences with amino acid deletions or insertions, or both. Guidance in determining which amino acid residues may be substituted, inserted, or deleted without abolishing the activity of the protein may be found using computer programs well known in the art. Variants may also include truncated forms derived from a wild-type P450 fatty acid decarboxylase enzyme, such as for example, a protein with a truncated N-terminus. Variants may also include forms derived by adding an extra amino acid sequence to a wild-type protein, such as for example, an N-terminal tag, a C-terminal tag or an insertion in the middle of the protein sequence.

As used herein, “reference sequence” refers to a defined sequence to which another sequence is compared. A reference sequence may be a subset of a larger sequence, for example, a segment of a full-length gene or polypeptide sequence. Generally, a reference sequence is at least about 20 nucleotide or amino acid residues in length, at least about 25 residues in length, at least about 50 residues in length, or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides over a comparison window to identify and compare local regions of sequence similarity. The term “reference sequence” is not intended to be limited to wild-type sequences, and can include engineered or altered sequences. For example, in embodiments, a “reference sequence” can be a previously engineered or altered amino acid sequence.

As used herein, “comparison window” refers to a conceptual segment of at least about about 20 contiguous nucleotide positions or amino acids residues wherein a sequence may be compared to a reference sequence of at least about 20 contiguous nucleotides or amino acids and wherein the portion of the sequence in the comparison window may comprise additions or deletions (i.e., gaps) of 20 percent or less as compared to the reference sequence (which does not comprise additions or deletions) for optimal alignment of the two sequences. The comparison window can be longer than 20 contiguous residues, and includes, optionally 30, 40, 50, 100, or longer windows.

As used herein, “corresponding to”, “reference to” or “relative to” when used in the context of the numbering of a given amino acid or polynucleotide sequence refers to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of an engineered P450 fatty acid decarboxylase, can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned.

As used herein, “increased enzymatic activity” and “increased activity” refer to an improved property of a wild-type or an engineered enzyme, which can be represented by an increase in specific activity (e.g., product produced/time/weight protein) or an increase in percent conversion of the substrate to the product (e.g., percent conversion of starting amount of substrate to product in a specified time period using a specified amount of P450 fatty acid decarboxylase) as compared to a reference enzyme. Any property relating to enzyme activity may be affected, including the classical enzyme properties of Km, Vmax or kcat, changes of which can lead to increased enzymatic activity. The P450 fatty acid decarboxylase activity can be measured by any one of standard assays used for measuring P450 fatty acid decarboxylases, such as change in substrate or product concentration. Comparisons of enzyme activities are made using a defined preparation of enzyme, a defined assay under a set condition, and one or more defined substrates, as further described in detail herein. Generally, when enzymes in cell lysates are compared, the numbers of cells and the amount of protein assayed are determined as well as use of identical expression systems and identical host cells to minimize variations in amount of enzyme produced by the host cells and present in the lysates.

As used herein, “conversion” refers to the enzymatic transformation of a substrate to the corresponding product.

As used herein “percent conversion” refers to the percent of the substrate that is converted to the product within a period of time under specified conditions. Thus, for example, the “enzymatic activity” or “activity” of a P450 fatty acid decarboxylase polypeptide can be expressed as “percent conversion” of the substrate to the product.

As used herein, “amino acid difference” or “residue difference” refers to a difference in the amino acid residue at a position of a polypeptide sequence relative to the amino acid residue at a corresponding position in a reference sequence. The positions of amino acid differences generally are referred to herein as “Xn”, where n refers to the corresponding position in the reference sequence upon which the residue difference is based. For example, a “residue difference at position X46 as compared to SEQ ID NO: 1” refers to a difference of the amino acid residue at the polypeptide position corresponding to position 46 of SEQ ID NO:1. Thus, if the reference polypeptide of SEQ ID NO:1 has a tyrosine at position 40, then a “residue difference at position X46 as compared to SEQ ID NO:1” refers to an amino acid substitution of any residue other than tyrosine at the position of the polypeptide corresponding to position 46 of SEQ ID NO:1. In most instances herein, the specific amino acid residue difference at a position is indicated as “XnY” where “Xn” specified the corresponding position as described above, and “Y” is the single letter identifier of the amino acid found in the engineered polypeptide (i.e., the different residue than in the reference polypeptide). In some instances, the present invention also provides specific amino acid differences denoted by the conventional notation “AnB”, where A is the single letter identifier of the residue in the reference sequence, “n” is the number of the residue position in the reference sequence, and B is the single letter identifier of the residue substitution in the sequence of the engineered polypeptide. In some instances, a polypeptide of the present invention can include at least one amino acid residue difference relative to a reference sequence, which is indicated by a list of the specified positions where residue differences are present relative to the reference sequence. In embodiments, where more than one amino acid can be used in a specific residue position of a polypeptide, the various amino acid residues that can be used are separated by a “/” (e.g., X46A/G). The present invention includes engineered polypeptide sequences comprising at least one amino acid difference that include either/or both conservative and non-conservative amino acid substitutions. The amino acid sequences of the specific recombinant P450 fatty acid decarboxylase polypeptides included in the Sequence Listing of the present invention include an initiating methionine (M) residue (i.e., M represents residue position 1). The skilled artisan, however, understands that this initiating methionine residue can be removed by biological processing machinery, such as in a host cell or in vitro translation system, to generate a mature protein lacking the initiating methionine residue, but otherwise retaining the enzyme's properties. Consequently, the term “amino acid residue difference relative to SEQ ID NO:1 at position Xn” as used herein may refer to position “Xn” or to the corresponding position (e.g., position (X−1)n) in a reference sequence that has been processed so as to lack the starting methionine.

The term “amino acid substitution set” or “substitution set” refers to a group of amino acid substitutions in a polypeptide sequence, as compared to a reference sequence. A substitution set can have 2, 3, 4, 5, 6, 7, 8, 9, 10, 11, 12, 13, 14, 15, or more amino acid substitutions. In embodiments, a substitution set refers to the set of amino acid substitutions that is present in any of the variant P450 fatty acid decarboxylases.

As used herein, the phrase “conservative amino acid substitutions” refers to the interchangeability of residues having similar side chains, and thus typically involves substitution of the amino acid in the polypeptide with amino acids within the same or similar defined class of amino acids. By way of example and not limitation, in embodiments, an amino acid with an aliphatic side chain is substituted with another aliphatic amino acid (e.g., alanine, valine, leucine, and isoleucine); an amino acid with a hydroxyl side chain is substituted with another amino acid with a hydroxyl side chain (e.g., serine and threonine); an amino acid having an aromatic side chains is substituted with another amino acid having an aromatic side chain (e.g., phenylalanine, tyrosine, tryptophan, and histidine); an amino acid with a basic side chain is substituted with another amino acid with a basic side chain (e.g., lysine and arginine); an amino acid with an acidic side chain is substituted with another amino acid with an acidic side chain (e.g., aspartic acid or glutamic acid); and/or a hydrophobic or hydrophilic amino acid is replaced with another hydrophobic or hydrophilic amino acid, respectively. The appropriate classification of any amino acid or residue will be apparent to those of skill in the art, especially in light of the detailed invention provided herein.

As used herein, the phrase “non-conservative substitution” refers to substitution of an amino acid in the polypeptide with an amino acid with significantly differing side chain properties. Non-conservative substitutions may use amino acids between, rather than within, the defined groups and affects (a) the structure of the peptide backbone in the area of the substitution (e.g., proline for glycine) (b) the charge or hydrophobicity, or (c) the bulk of the side chain. By way of example and not limitation, an exemplary non-conservative substitution can be an acidic amino acid substituted with a basic or aliphatic amino acid; an aromatic amino acid substituted with a small amino acid; and a hydrophilic amino acid substituted with a hydrophobic amino acid.

As used herein, “deletion” refers to modification of the polypeptide by removal of one or more amino acids from the reference polypeptide. Deletions can comprise removal of 1 or more amino acids, 2 or more amino acids, 5 or more amino acids, 10 or more amino acids, 15 or more amino acids, or 20 or more amino acids, up to 10% of the total number of amino acids, or up to 20% of the total number of amino acids making up the polypeptide while retaining enzymatic activity and/or retaining the improved properties of an engineered enzyme. Deletions can be directed to the internal portions and/or terminal portions of the polypeptide. In various embodiments, the deletion can comprise a continuous segment or can be discontinuous.

As used herein, “insertion” refers to modification of the polypeptide by addition of one or more amino acids to the reference polypeptide. In embodiments, the improved engineered P450 fatty acid decarboxylase enzymes comprise insertions of one or more amino acids to the naturally occurring P450 fatty acid decarboxylase polypeptide as well as insertions of one or more amino acids to engineered P450 fatty acid decarboxylase polypeptides. Insertions can be in the internal portions of the polypeptide, or to the carboxy or amino terminus. Insertions as used herein include fusion proteins as is known in the art. The insertion can be a contiguous segment of amino acids or separated by one or more of the amino acids in the naturally occurring polypeptide.

As used herein, “fragment” refers to a polypeptide that has an amino-terminal and/or carboxy-terminal deletion, but where the remaining amino acid sequence is identical to the corresponding positions in the sequence. Fragments can typically have about 80%, about 90%, about 95%, about 98%, or about 99% of the full-length P450 fatty acid decarboxylase polypeptide, for example, the polypeptide of SEQ ID NO: 1. In embodiments, the fragment is “biologically active” (i.e., it exhibits the same enzymatic activity as the full-length sequence).

A “functional fragment”, or a “biologically active fragment”, used interchangeably, herein refers to a polypeptide that has an amino-terminal and/or carboxy-terminal deletion(s) and/or internal deletions, but where the remaining amino acid sequence is identical to the corresponding positions in the sequence to which it is being compared and that retains substantially all of the activity of the full-length polypeptide.

As used herein, “isolated polypeptide” refers to a polypeptide which is substantially separated from other contaminants that naturally accompany it (e.g., protein, lipids, and polynucleotides). The term embraces polypeptides which have been removed or purified from their naturally-occurring environment or expression system (e.g., host cell or in vitro synthesis). The improved P450 fatty acid decarboxylase enzymes may be present within a cell, present in the cellular medium, or prepared in various forms, such as lysates or isolated preparations. As such, in embodiments, the wild-type or engineered P450 fatty acid decarboxylase polypeptides of the present invention can be an isolated polypeptide.

As used herein, “substantially pure polypeptide” refers to a composition in which the polypeptide species is the predominant species present (i.e., on a molar or weight basis it is more abundant than any other individual macromolecular species in the composition), and is generally a substantially purified composition when the object species comprises at least about 50 percent of the macromolecular species present by mole or % weight. Generally, a substantially pure wild-type or engineered P450 fatty acid decarboxylase polypeptide composition will comprise about 60% or more, about 70% or more, about 80% or more, about 90% or more, about 91% or more, about 92% or more, about 93% or more, about 94% or more, about 95% or more, about 96% or more, about 97% or more, about 98% or more, or about 99% of all macromolecular species by mole or % weight present in the composition. Solvent species, small molecules (<500 Daltons), and elemental ion species are not considered macromolecular species. In embodiments, the isolated improved P450 fatty acid decarboxylase polypeptide is a substantially pure polypeptide composition.

As used herein, when used with reference to a nucleic acid or polypeptide, the term “heterologous” refers to a sequence that is not normally expressed and secreted by an organism (e.g., a wild-type organism). In embodiments, the term encompasses a sequence that comprises two or more subsequences, which are not found in the same relationship to each other as normally found in nature, or is recombinantly engineered so that its level of expression, or physical relationship to other nucleic acids or other molecules in a cell, or structure, is not normally found in nature. For instance, a heterologous nucleic acid is typically recombinantly produced, having two or more sequences from unrelated genes arranged in a manner not found in nature (e.g., a nucleic acid open reading frame (ORF) of the invention operatively linked to a promoter sequence inserted into an expression cassette, such as a vector). In embodiments, “heterologous polynucleotide” refers to any polynucleotide that is introduced into a host cell by laboratory techniques, and includes polynucleotides that are removed from a host cell, subjected to laboratory manipulation, and then reintroduced into a host cell.

As used herein, “codon optimized” refers to changes in the codons of the polynucleotide encoding a protein to those preferentially used in a particular organism such that the encoded protein is efficiently expressed in the organism of interest. In embodiments, the polynucleotides encoding the P450 fatty acid decarboxylase enzymes may be codon optimized for optimal production from the host organism selected for expression.

As used herein, “suitable reaction conditions” refer to those conditions in the biocatalytic reaction solution (e.g., ranges of enzyme loading, substrate loading, temperature, pH, buffers, co-solvents, etc.) under which a P450 fatty acid decarboxylase polypeptide of the present invention is capable of converting a substrate compound to a product compound (e.g., conversion of one compound to another compound).

As used herein, “substrate” in the context of a biocatalyst mediated process refers to the compound or molecule acted on by the biocatalyst.

As used herein “product” in the context of a biocatalyst mediated process refers to the compound or molecule resulting from the action of the biocatalyst.

All percentages and ratios used hereinafter are by weight of total composition, unless otherwise indicated. All percentages, ratios, and levels of ingredients referred to herein are based on the actual amount of the ingredient, and do not include solvents, fillers, or other materials with which the ingredient may be combined as a commercially available product, unless otherwise indicated.

P450 Fatty Acid Decarboxylases

P450 fatty acid decarboxylases are enzymes that belong to the cytochrome P450 family CYP152 and catalyze the decarboxylation of fatty acids to alkenes utilizing hydrogen peroxide as co-substrate and heme as a cofactor. The most well studied member of this family is OleTJE (SEQ ID NO: 1), an enzyme endogenous to Jeotgalicoccus sp. 8456 (J. Belcher et al., J. Biol. Chem. (2014), 289, 10: 6535-6550) and is classified as CYP152L1. Variants of OleTJE with fused domains are capable of using molecular oxygen as co-substrate in the presence of an additional co-substrate (Y. Liu et al., Biotechnol. Biofuels (2014) 7: 28).

CYP152 enzymes related to OleTJE may exhibit some decarboxylase activity. For instance, variants CYP152A1, CYP152A2, and CYP152B1 are described as being more effective at catalyzing alpha or beta hydroxylation of fatty acids, with decarboxylation as a side reaction. However, protein engineering has been proven to convert such enzymes into more effective fatty acid decarboxylases, for example by making the Gln85His substitution in CYP152A1.

The present invention provides consumer product compositions comprising P450 fatty acid decarboxylases having increased enzymatic activity for long-chain fatty acid substrates, such as oleic acid, as compared to the well-known naturally occurring wild-type fatty acid decarboxylases reported previously in the art (e.g. OleTJE, SEQ ID NO: 1). Surprisingly, Applicant has found that P450 fatty acid decarboxylases comprising specific amino acid residues at certain positions (e.g. 40, 46, 74, 252, or 317) have an increased enzymatic activity towards long chain fatty acids, such as oleic acid in comparison to the well-known OleTJE (SEQ ID NO: 1) and that these decarboxylases can provide a benefit when formulated in consumer products, such as detergents.

In embodiments of the current invention, a consumer product composition comprises a P450 fatty acid decarboxylase; wherein said decarboxylase comprises an amino acid selected from the group consisting of: a) valine, isoleucine, or leucine at position 40, b) alanine, glycine, or valine at position 46, c) valine at position 74, d) lysine at position 252, e) isoleucine, leucine, methionine, or valine at position 317, and combinations thereof; wherein said positions are numbered with reference to SEQ ID NO: 1.

In embodiments of the current invention, a consumer product composition comprises a P450 fatty acid decarboxylase comprising an isoleucine, leucine, or valine, at position 40; wherein said positions are numbered with reference to SEQ ID NO: 1. Non-limiting examples of P450 fatty acid decarboxylases comprising an isoleucine at position 40, wherein said position is numbered with reference to SEQ ID NO: 1, are SEQ ID NO: 22, 30, and 32. Non-limiting examples of P450 fatty acid decarboxylases comprising a leucine at position 40, wherein said position is numbered with reference to SEQ ID NO: 1, are SEQ ID NO: 29, 31, 33, 34, 35, 36, 37, 38, 64, 65, 66, and 121. Non-limiting examples of P450 fatty acid decarboxylases comprising a valine at position 40, wherein said position is numbered with reference to SEQ ID NO: 1, are SEQ ID NO: 2, 3, 4, 5, 6, 7, 8, 9, 10, 11, 12, 13, 14, 15, 16, 17, 18, 19, 20, 21, 23, 24, 25, 26, 27, 28, 122, 129, 148, 149, 150, and 151.

In embodiments of the current invention, a consumer product composition comprises a P450 fatty acid decarboxylase comprising an alanine, glycine, or valine at position 46; wherein said positions are numbered with reference to SEQ ID NO: 1. Non-limiting examples of P450 fatty acid decarboxylases comprising an alanine at position 46, wherein said position is numbered with reference to SEQ ID NO: 1, are SEQ ID NO: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 129, and 151. Non-limiting examples of P450 fatty acid decarboxylases comprising an valine at position 46, wherein said position is numbered with reference to SEQ ID NO: 1, are SEQ ID NO: 39, 40, 41, 42, 43, 44, 45, 46, 47, 48, 49, 50, 51, 52, 53, 54, 55, 56, 57, 58, 59, 60, 61, 62, 63, 64, 65, and 66.

In embodiments of the current invention, a consumer product composition comprises a P450 fatty acid decarboxylase comprising a valine at position 74; wherein said positions are numbered with reference to SEQ ID NO: 1. Non-limiting examples of P450 fatty acid decarboxylases comprising a valine at position 74, wherein said position is numbered with reference to SEQ ID NO: 1, are SEQ ID NO: 2, 3, 4, 5, 6, 7, 8, 9, 10, 11, 12, 13, 14, 15, 16, 17, 18, 19, 20, 21, 42, 53, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, and 104.

In embodiments of the current invention, a consumer product composition comprises a P450 fatty acid decarboxylase comprising a lysine at position 252; wherein said positions are numbered with reference to SEQ ID NO: 1. Non-limiting examples of P450 fatty acid decarboxylases comprising a lysine at position 252, wherein said position is numbered with reference to SEQ ID NO: 1, are SEQ ID NO: 2, 3, 4, 5, 6, 7, 8, 9, 10, 11, 12, 13, 14, 15, and 151.

In embodiments of the current invention, a consumer product composition comprises a P450 fatty acid decarboxylase comprising an isoleucine, leucine, methionine, or valine at position 317; wherein said positions are numbered with reference to SEQ ID NO: 1. Non-limiting examples of P450 fatty acid decarboxylases comprising an isoleucine at position 317, wherein said position is numbered with reference to SEQ ID NO: 1, are SEQ ID NO: 9, 10, 11, 12, 13, 14, 15, 16, 17, 18, 19, 20, 22, 23, 24, 54, 55, 56, 57, 58, 59, 60, 61, 62, 63, 81, and 129. Non-limiting examples of P450 fatty acid decarboxylases comprising a leucine at position 317, wherein said position is numbered with reference to SEQ ID NO: 1, are SEQ ID NO: 2, 3, 4, 5, 6, 7, 8, 42, 64, 65, 66, 84, 86, 87, 88, 89, 90, 91, 94, 95, 96, 97, 98, 99, 102, 103, 104, 105, 106, 107, 108, 109, 110, 111, 112, 113, 122, and 151. A non-limiting example of P450 fatty acid decarboxylases comprising a methionine at position 317, wherein said position is numbered with reference to SEQ ID NO: 1; is SEQ ID NO: 121. Non-limiting examples of P450 fatty acid decarboxylases comprising a valine at position 317, wherein said position is numbered with reference to SEQ ID NO: 1; are SEQ ID NO: 21, 25, 26, 27, 28, 39, 40, 41, 43, 44, 45, 46, 47, 49, 51, 52, 67, 68, 69, 70, 71, 72, 73, 74, 75, 76, 77, 78, 79, 80, 82, 83, 93, 114, 115, 116, 117, 118, 119, 123, 124, 125, 126, 127, 128, 130, 131, 132, 133, 134, 135, 136, 137, 138, 139, 140, 141, 142, 143, 144, 145, 146, and 147.

In embodiments, the decarboxylases have an increased enzymatic activity for oleic acid of at least about 2-fold, 3-fold, 4-fold, 5-fold, 10-fold, 20-fold, 30-fold, 40-fold, 50-fold, 150-fold, 500-fold or more relative to the activity of wild-type decarboxylase (SEQ ID NO: 1) under suitable reaction conditions.

In embodiments, the consumer product composition comprises a P450 fatty acid decarboxylase; wherein said decarboxylase comprises a polypeptide sequence having at least about 80%, at least about 90%, at least about 95%, at least about 98%, at least about 100% identity to one or more sequences selected from the group consisting of SEQ ID NO: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 156, 157, 158, and their functional fragments thereof. In embodiments, the consumer production composition comprises a P450 fatty acid decarboxylase; wherein said decarboxylase comprises a polypeptide sequence having at least about 80%, at least about 90%, at least about 95%, at least about 98%, at least about 100% identity to one or more sequences selected from the group consisting of SEQ ID NO: 2, 22, 44, 60, 65, 71, 83, 117, 121, 122, 156, and their functional fragments thereof. In embodiments, the consumer production composition comprises a P450 fatty acid decarboxylase; wherein said decarboxylase comprises a polypeptide sequence having at least about 80%, at least about 90%, at least about 95%, at least about 98%, at least about 100% identity to one or more sequences selected from the group consisting of SEQ ID NO: 2, 60, 65, 71, 83, 122, and their functional fragments thereof. In embodiments, the consumer production composition comprises a P450 fatty acid decarboxylase; wherein said decarboxylase comprises a polypeptide sequence having at least about 80%, at least about 90%, at least about 95%, at least about 98%, at least about 100% identity to one or more sequences selected from the group consisting of SEQ ID NO: 2, 122, and their functional fragments thereof. In embodiments, the consumer production composition comprises a P450 fatty acid decarboxylase; wherein said decarboxylase comprises a polypeptide sequence having at least about 80%, at least about 90%, at least about 95%, at least about 98%, at least about 100% identity to one or more sequences selected from the group consisting of SEQ ID NO: 2. In embodiments, the consumer production composition comprises a P450 fatty acid decarboxylase; wherein said decarboxylase comprises a polypeptide sequence having at least about 90%, at least about 95%, at least about 98%, at least about 100% identity to one or more sequences selected from the group consisting of SEQ ID NO: 2, 3, 4, 5, 6, 7, 8, 9, 10, 11, 12, 13, 14, 15, 22, 23, 24, 39, 40, 41, 43, 44, 44, 45, 46, 47, 48, 49, 50, 54, 55, 56, 57, 58, 59, 60, 60, 61, 62, 63, 64, 65, 67, 68, 69, 70, 71, 71, 72, 73, 74, 75, 76, 77, 78, 79, 80, 81, 82, 83, 85, 114, 115, 116, 117, 117, 118, 119, 121, 122, 123, 124, 125, 126, 127, 128, 129, 130, 131, 132, 133, 134, 135, 136, 137, 138, 139, 140, 141, 142, 143, 144, 145, 146, 151, 156, 157, and their functional fragments thereof. In embodiments, the consumer production composition comprises a P450 fatty acid decarboxylase; wherein said decarboxylase comprises a polypeptide sequence having at least about 90%, at least about 95%, at least about 98%, at least about 100% identity to one or more sequences selected from the group consisting of SEQ ID NO: 2, 3, 5, 4, 7, 6, 8, 9, 10, 11, 12, 13, 14, 15, 122, 151, and their functional fragments thereof. Suitable examples of P450 fatty acid decarboxylases with at least about 80% identity to SEQ ID NO: 2 are SEQ ID NO: 3, 5, 4, 7, 6, 8, 9, 10, 11, 12, 13, 14, 15, and 151. Suitable examples of P450 fatty acid decarboxylases with at least about 80% identity to SEQ ID NO: 22 are SEQ ID NO: 23, 24, 129. Suitable examples of P450 fatty acid decarboxylases with at least about 80% identity to SEQ ID NO: 44 are SEQ ID NO: 39, 40, 41, 43, 44, 45, 46, 47, 48, 49, 50, 85, 126, 127, 128, 130, 131, 132, 133, 134, 135, 136, 137, 138, 139, 140, 141, 142, 143, 144, 145, and 146. Suitable examples of P450 fatty acid decarboxylases with at least about 80% identity to SEQ ID NO: 60 are SEQ ID NO: 54, 55, 56, 57, 58, 59, 61, 62, and 63. A suitable example of P450 fatty acid decarboxylases with at least about 80% identity to SEQ ID NO: 65 is SEQ ID NO: 64. Suitable examples of P450 fatty acid decarboxylases with at least about 80% identity to SEQ ID NO: 71 are SEQ ID NO: 67, 68, 69, 70, 72, 73, 74, 75, 76, 77, 78, 79, 80, 81, 123, 124, and 125. Suitable examples of P450 fatty acid decarboxylases with at least about 80% identity to SEQ ID NO: 117 are SEQ ID NO: 114, 115, 116, 118, and 119. Identity, or homology, percentages as mentioned herein in respect of the present invention are those that can be calculated, for example, with AlignX obtainable from Thermo Fischer Scientific or with the alignment tool from Uniprot (https://www.uniprot.org/align/). Alternatively, a manual alignment can be performed. For enzyme sequence comparison the following settings can be used: Alignment algorithm: Needleman and Wunsch, J. Mol. Biol. 1970, 48: 443-453. As a comparison matrix for amino acid similarity the Blosum62 matrix is used (Henikoff S. and Henikoff J. G., P.N.A.S. USA 1992, 89: 10915-10919). The following gap scoring parameters are used: Gap penalty: 12, gap length penalty: 2, no penalty for end gaps.

A given sequence is typically compared against the full-length sequence or fragments of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 156, 157, or 158 to obtain a score. In embodiments, polypeptides of the present disclosure include polypeptides containing an amino acid sequence having at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or 100% identity to the amino acid sequence of any one of SEQ ID NO: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 156, 157, and 158. Polypeptides of the disclosure also include polypeptides having at least about 10, at least about 12, at least about 14, at least about 16, at least about 18, at least about 20, at least about 30, at least about 40, at least about 50, at least about 60, at least about 70, or at least about 80 consecutive amino acids of the amino acid sequence of any one of SEQ ID NO: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 156, 157, and 158.

The present invention also includes variants of P450 fatty acid decarboxylases, as previously described. Variants of P450 fatty acid decarboxylases include polypeptide sequences resulting from modification of a wild-type P450 fatty acid decarboxylase at one or more amino acids. A variant includes a “modified enzyme” or a “mutant enzyme” which encompasses proteins having at least one substitution, insertion, and/or deletion of an amino acid. A modified enzyme may have 1, 2, 3, 4, 5, 6, 7, 8, 9 or 10 or more amino acid modifications (selected from substitutions, insertions, deletions and combinations thereof).

The variants may have “conservative” substitutions. Suitable examples of conservative substitution includes one conservative substitution in the enzyme, such as a conservative substitution in SEQ ID NO: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 156, 157, 158, and their functional fragments thereof. Other suitable examples include 10 or fewer conservative substitutions in the protein, such as five or fewer. An enzyme of the invention may therefore include 1, 2, 3, 4, 5, 6, 7, 8, 9, 10 or more conservative substitutions. An enzyme can be produced to contain one or more conservative substitutions by manipulating the nucleotide sequence that encodes that enzyme using, for example, standard procedures such as site-directed mutagenesis or PCR. Examples of amino acids which may be substituted for an original amino acid in an enzyme and which are regarded as conservative substitutions include: Ser for Ala; Lys for Arg; Gln or His for Asn; Glu for Asp; Asn for Gln; Asp for Glu; Pro for Gly; Asn or Gln for His; Leu or Val for Ile; Ile or Val for Leu; Arg or Gln for Lys; Leu or Ile for Met; Met, Leu or Tyr for Phe; Thr for Ser; Ser for Thr; Tyr for Trp; Trp or Phe for Tyr; and Ile or Leu for Val.

In embodiments of the present invention, the variant of the P450 fatty acid decarboxylase comprises a polypeptide sequence comprising at least one amino acid substitution at positions selected from the group consisting of: 40, 46, 74, 79, 245, 246, 252, 253, 256, 286, 317, 365, and combinations thereof; wherein said positions are numbered with reference to SEQ ID NO: 1. In embodiments of the present invention, the variant of the P450 fatty acid decarboxylase comprises a polypeptide sequence comprising at least one amino acid substitution, deletion, or insertion at positions selected from the group consisting of: 174, 175, 176, 177, 178, 179, 180, 181, and combinations thereof; wherein said positions are numbered with reference to SEQ ID NO: 1.

In embodiments, the variant of the P450 fatty acid decarboxylase comprises a polypeptide sequence comprising at least one amino acid substitution selected from the group consisting of: F79A, R245P, P246R, K252Q, F253A, F256A, R286Q, and combinations thereof; wherein said positions are numbered with reference to SEQ ID NO: 1.

In embodiments, the variant of the P450 fatty acid decarboxylase comprises a polypeptide sequence comprising at least one amino acid substitution at positions selected from the group consisting of: 77, 78, 79, 85, 166, 169, 170, 190, 193, 238, 240, 241, 242, 243, 244, 245, 246, 247, 248, 249, and combinations thereof; wherein said positions are numbered with reference to SEQ ID NO: 1; and wherein said amino acid substitution is for an amino acid selected from the group consisting of aspartate and glutamate.

In some embodiments the variant of the P450 fatty acid decarboxylase comprises a polypeptide sequence comprising at least one amino acid substitution selected from the group consisting of: F80A, R246P, P247R, K253Q, F254A, F257A, R288Q, and combinations thereof; wherein said positions are numbered with reference to SEQ ID NO: 2.

It is important that variants of enzymes retain and preferably improve the ability of the wild-type protein to catalyze the conversion of the fatty acids. Some performance drop in a given property of variants may of course be tolerated, but the variants should retain and preferably improve suitable properties for the relevant application for which they are intended. Screening of variants of one of the wild-types can be used to identify whether they retain and preferably improve appropriate properties.

The decarboxylase polypeptides described herein are not restricted to the genetically encoded amino acids. Thus, in addition to the genetically encoded amino acids, the polypeptides described herein may be comprised, either in whole or in part, of naturally-occurring and/or synthetic non-encoded amino acids. Certain commonly encountered non-encoded amino acids of which the polypeptides described herein may be comprised include, but are not limited to: the D-stereoisomers of the genetically-encoded amino acids; 2,3-diaminopropionic acid (Dpr); α-aminoisobutyric acid (Aib); ε-aminohexanoic acid (Aha); δ-aminovaleric acid (Ava); N-methylglycine or sarcosine (MeGly or Sar); ornithine (Orn); citrulline (Cit); t-butylalanine (Bua); t-butylglycine (Bug); N-methylisoleucine (MeIle); phenylglycine (Phg); cyclohexylalanine (Cha); norleucine (Nle); naphthylalanine (Nal); 2-chlorophenylalanine (Oct); 3-chlorophenylalanine (Mcf); 4-chlorophenylalanine (Pcf); 2-fluorophenylalanine (Off); 3-fluorophenylalanine (Mff); 4-fluorophenylalanine (Pff); 2-bromophenylalanine (Obf); 3-bromophenylalanine (Mbf); 4-bromophenylalanine (Pbf); 2-methylphenylalanine (Omf); 3-methylphenylalanine (Mmf); 4-methylphenylalanine (Pmf); 2-nitrophenylalanine (Onf); 3-nitrophenylalanine (Mnf); 4-nitrophenylalanine (Pnf); 2-cyanophenylalanine (Ocf); 3-cyanophenylalanine (Mcf); 4-cyanophenylalanine (Pcf); 2-trifluoromethylphenylalanine (Otf); 3-trifluoromethylphenylalanine (Mtf); 4-trifluoromethylphenylalanine (Ptf); 4-aminophenylalanine (Paf); 4-iodophenylalanine (Pif); 4-aminomethylphenylalanine (Pamf); 2,4-dichlorophenylalanine (Opcf); 3,4-dichlorophenylalanine (Mpcf); 2,4-difluorophenylalanine (Opff); 3,4-difluorophenylalanine (Mpff); pyrid-2-ylalanine (2pAla); pyrid-3-ylalanine (3pAla); pyrid-4-ylalanine (4pAla); naphth-1-ylalanine (1nAla); naphth-2-ylalanine (2nAla); thiazolylalanine (taAla); benzothienylalanine (bAla); thienylalanine (tAla); furylalanine (fAla); homophenylalanine (hPhe); homotyrosine (hTyr); homotryptophan (hTrp); pentafluorophenylalanine (5ff); styrylkalanine (sAla); authrylalanine (aAla); 3,3-diphenylalanine (Dfa); 3-amino-5-phenylpentanoic acid (Afp); penicillamine (Pen); 1,2,3,4-tetrahydroisoquinoline-3-carboxylic acid (Tic); β-2-thienylalanine (Thi); methionine sulfoxide (Mso); N(w)-nitroarginine (nArg); homolysine (hLys); phosphonomethylphenylalanine (pmPhe); phosphoserine (pSer); phosphothreonine (pThr); homoaspartic acid (hAsp); homoglutamic acid (hGlu); 1-aminocyclopent-(2 or 3)-ene-4 carboxylic acid; pipecolic acid (PA), azetidine-3-carboxylic acid (ACA); 1-aminocyclopentane-3-carboxylic acid; allylglycine (aOly); propargylglycine (pgGly); homoalanine (hAla); norvaline (nVal); homoleucine (hLeu), homovaline (hVal); homoisoleucine (hIle); homoarginine (hArg); N-acetyl lysine (AcLys); 2,4-diaminobutyric acid (Dbu); 2,3-diaminobutyric acid (Dab); N-methylvaline (MeVal); homocysteine (hCys); homoserine (hSer); hydroxyproline (Hyp) and homoproline (hPro). Additional non-encoded amino acids of which the polypeptides described herein may be comprised will be apparent to those of skill in the art. These amino acids may be in either the L- or D-configuration.

The invention also includes variants in the form of truncated forms or fragments derived from a wild-type enzyme, such as a protein with a truncated N-terminus or a truncated C-terminus. In embodiments, the present invention also provides variants of decarboxylase enzymes that comprise a fragment of any of the decarboxylase polypeptides described herein that retain the functional decarboxylase activity and/or an improved property of an engineered decarboxylase polypeptide. Accordingly, in embodiments, the present invention provides a polypeptide fragment having decarboxylase activity (e.g., capable of converting substrate to product under suitable reaction conditions), wherein the fragment comprises at least about 80%, 90%, 95%, 98%, or 99% of a full-length amino acid sequence of an engineered polypeptide of the present invention.

In embodiments, the present invention provides a decarboxylase enzyme having an amino acid sequence comprising an insertion as compared to any one of the decarboxylase polypeptide sequences described herein. Thus, for each and every embodiment of the decarboxylase polypeptides of the invention, the insertions can comprise one or more amino acids, 2 or more amino acids, 3 or more amino acids, 4 or more amino acids, 5 or more amino acids, 6 or more amino acids, 8 or more amino acids, 10 or more amino acids, 15 or more amino acids, or 20 or more amino acids, where the associated functional activity and/or improved properties of the decarboxylase described herein is maintained. The insertions can be to amino or carboxy terminus, or internal portions of the decarboxylase polypeptide. The invention also includes variants derived by adding an extra amino acid sequence, such as an N-terminal tag or a C-terminal tag. Non-limiting examples of tags are maltose binding protein (MBP) tag, glutathione S-transferase (GST) tag, thioredoxin (Trx) tag, His-tag, and any other tags known by those skilled in art. Tags can be used to improve solubility and expression levels during fermentation or as a handle for enzyme purification.

Enzymes can also be modified by a variety of chemical techniques to produce derivatives having essentially the same or preferably improved activity as the unmodified enzymes, and optionally having other desirable properties. For example, carboxylic acid groups of the protein, whether carboxyl-terminal or side chain, may be provided in the form of a salt of a pharmaceutically-acceptable cation or esterified, for example to form a C1-C6 alkyl ester, or converted to an amide, for example of formula CONR1R2 wherein R1 and R2 are each independently H or C1-C6 alkyl, or combined to form a heterocyclic ring, such as a 5- or 6-membered ring. Amino groups of the enzyme, whether amino-terminal or side chain, may be in the form of a pharmaceutically-acceptable acid addition salt, such as the HCl, HBr, acetic, benzoic, toluene sulfonic, maleic, tartaric and other organic salts, or may be modified to C1-C20 alkyl or dialkyl amino or further converted to an amide. Hydroxyl groups of the protein side chains may be converted to alkoxy or ester groups, for example C1-C20 alkoxy or C1-C20 alkyl ester, using well-recognized techniques. Phenyl and phenolic rings of the protein side chains may be substituted with one or more halogen atoms, such as F, Cl, Br or I, or with C1-C20 alkyl, C1-C20 alkoxy, carboxylic acids and esters thereof, or amides of such carboxylic acids. Methylene groups of the protein side chains can be extended to homologous C2-C4 alkylenes. Thiols can be protected with any one of a number of well-recognized protecting groups, such as acetamide groups. Those skilled in the art will also recognize methods for introducing cyclic structures into the proteins of this disclosure to select and provide conformational constraints to the structure that result in enhanced stability.

In embodiments, the enzymes can be provided on a solid support, such as a membrane, resin, solid carrier, or other solid phase material. A solid support can be composed of organic polymers such as polystyrene, polyethylene, polypropylene, polyfluoroethylene, polyethyleneoxy, and polyacrylamide, as well as co-polymers and grafts thereof. A solid support can also be inorganic, such as glass, silica, controlled pore glass (CPG), reverse phase silica or metal, such as gold or platinum. The configuration of a solid support can be in the form of beads, spheres, particles, granules, a gel, a membrane or a surface. Surfaces can be planar, substantially planar, or non-planar. Solid supports can be porous or non-porous, and can have swelling or non-swelling characteristics. A solid support can be configured in the form of a well, depression, or other container, vessel, feature, or location.

In embodiments, the polypeptides having decarboxylase activity are bound or immobilized on the solid support such that they retain at least a portion of their improved properties relative to a reference polypeptide (e.g., SEQ ID NO: 1). Accordingly, it is further contemplated that any of the methods of using the decarboxylase polypeptides of the present invention can be carried out using the same decarboxylase polypeptides bound or immobilized on a solid support.

The decarboxylase polypeptide can be bound non-covalently or covalently. Various methods for conjugation and immobilization of enzymes to solid supports (e.g., resins, membranes, beads, glass, etc.) are well known in the art. Other methods for conjugation and immobilization of enzymes to solid supports (e.g., resins, membranes, beads, glass, etc.) are well known in the art (See, e.g., Yi et al., Proc. Biochem., 42: 895-898 [2007]; Martin et al., Appl. Microbiol. Biotechnol., 76: 843-851 [2007]; Koszelewski et al. J. Mol. Cat. B: Enz., 63: 39-44 [2010]; Truppo et al., Org. Proc. Res. Develop., published online: dx.doi.org/10.1021/op200157c; and Mateo et al., Biotechnol. Prog., 18:629-34 [2002], etc.). Solid supports useful for immobilizing the decarboxylase polypeptides of the present invention include, but are not limited to, beads or resins comprising polymethacrylate with epoxide functional groups, polymethacrylate with amino epoxide functional groups, styrene/DVB copolymer or polymethacrylate with octadecyl functional groups.

The enzymes may be incorporated into the consumer product compositions via an additive particle, such as an enzyme granule or in the form of an encapsulate or may be added in the form of a liquid formulation. Encapsulating the enzymes promote the stability of the enzymes in the composition and helps to counteract the effect of any hostile compounds present in the composition, such as bleach, protease, surfactant, chelant, etc. The P450 fatty acid decarboxylase enzymes may be the only enzymes in the additive particle or may be present in the additive particle in combination with one or more additional co-enzymes.

In embodiments, the consumer product composition comprises a P450 fatty acid decarboxylase, wherein said P450 fatty acid decarboxylase is present in an amount of from 0.0001 wt % to 1 wt %, preferably from 0.001 wt % to 0.2 wt %, by weight of the consumer product composition, based on active protein.

In embodiments, the consumer product further comprises one or more co-enzymes selected from the group consisting of: fatty-acid peroxidases (EC 1.11.1.3), unspecific peroxygenases (EC 1.11.2.1), plant seed peroxygenases (EC 1.11.2.3), fatty acid peroxygenases (EC1.11.2.4), linoleate diol synthases (EC 1.13.11.44), 5,8-linoleate diol synthases (EC 1.13.11.60 and EC 5.4.4.5), 7,8-linoleate diol synthases (EC 1.13.11.60 and EC 5.4.4.6), 9,14-linoleate diol synthases (EC 1.13.11.B1), 8,11-linoleate diol synthases, oleate diol synthases, other linoleate diol synthases, unspecific monooxygenase (EC 1.14.14.1), alkane 1-monooxygenase (EC 1.14.15.3), oleate 12-hydroxylases (EC 1.14.18.4), fatty acid amide hydrolases (EC 3.5.1.99), fatty acid photodecarboxylases (EC 4.1.1.106), oleate hydratases (EC 4.2.1.53), linoleate isomerases (EC 5.2.1.5), linoleate (10E,12Z)-isomerases (EC 5.3.3.B2), non-heme fatty acid decarboxylases (UndA-like), alpha-dioxygenases, amylases, lipases, proteases, cellulases, and mixtures thereof; preferably fatty-acid peroxidases (EC 1.11.1.3), unspecific peroxygenases (EC 1.11.2.1), plant seed peroxygenases (EC 1.11.2.3), and fatty acid peroxygenases (EC1.11.2.4), non-heme fatty acid decarboxylases (UndA-like), alpha-dioxygenases, and mixtures thereof.

Where necessary, the composition comprises, provides access to, or forms in situ any additional substrate necessary for the effective functioning of the enzyme. For example, molecular hydrogen peroxide can be provided as an additional substrate for P450 fatty acid decarboxylases. In embodiments, the consumer product composition may be supplemented with heme and/or a source of iron to enhance or facilitate the conversion of the fatty acids.

In embodiments, the P450 fatty acid decarboxylase comprises a heme cofactor selected from the group comprising: heme a, heme b, heme c, heme d, heme i, heme m, heme o, heme s, their derivatives, and mixtures thereof; preferably heme b. In other embodiments, the heme cofactor is covalently attached to the P450 fatty acid decarboxylases.

In some embodiments, the P450 fatty acid decarboxylase comprises a heme cofactor comprising: a) a porphyrin group and b) a metal. Non-limiting examples of porphyrin groups are: protoporphyrin IX, N-methyl protoporphyrin IX, protoporphyrin IX monomethyl ester, protoporphyrin IX dimethyl ester, protoporphyrin IX diamide, protoporphyrin IX bis thiosulfate, porphin, phthalocyanine, octaethylporphoyrin, tetraphenylporphyrin, and their derivatives; preferably protoporphyrin IX. Non-limiting examples of metals are: Mg, Al, V, Cr, Mn, Fe, Co, Ni, Cu, Zn, Ga, Ge, Mo, Tc, Ru, Rh, Pd, Ag, Cd, In, Sn, Sb, W, Re, Os, Ir, Pt, Au, Hg, Tl, Pb, Bi, and mixtures thereof; preferably Fe. In some embodiments, the P450 fatty acid decarboxylase comprises a heme cofactor comprising a cation selected from the group comprising: Mg²⁺, Cr³⁺, Mn³⁺, Fe³⁺, Co³⁺, Ni²⁺, Cu²⁺, Zn²⁺, Ga³⁺, Rh²⁺, Pd²⁺, Ag²⁺, In³⁺, Sn⁴⁺, VO²⁺, and mixtures thereof; preferably Fe³⁺. In some embodiments, the P450 fatty acid decarboxylase comprises a heme cofactor comprising an axially bound ligand. Non-limiting examples of ligands are: chloride, methyl group, carbonyl group, hydroxide group, and tetrahydrofuran.

Polynucleotides and Plasmids

In another aspect, the present invention provides polynucleotides encoding the decarboxylase enzymes. The polynucleotides may be operatively linked to one or more heterologous regulatory sequences that control gene expression to create a recombinant polynucleotide capable of expressing the polypeptide. Expression constructs containing a heterologous polynucleotide encoding the decarboxylase can be introduced into appropriate host cells to express the corresponding decarboxylase polypeptide.

Due to the degeneracy of the genetic code, where the same amino acids are encoded by alternative or synonymous codons, a large number of nucleic acids that encode the decarboxylase enzymes disclosed herein can be produced. Those skilled in the art could make any number of different nucleic acids by simply modifying the sequence of one or more codons in a way which does not change the amino acid sequence of the protein. In this regard, the present invention specifically contemplates each and every possible variation of polynucleotides that could be made by selecting combinations based on the possible codon choices, and all such variations are to be considered specifically disclosed for any polypeptide disclosed herein. In various embodiments, the codons are preferably selected to fit the host cell in which the protein is being produced. For example, preferred codons used in bacteria are used to express the gene in bacteria; preferred codons used in yeast are used for expression in yeast; and preferred codons used in mammals are used for expression in mammalian cells.

The polynucleotides encoding the enzyme can be prepared by standard methods, such as solid-phase methods. In embodiments, fragments of up to about 100 bases can be individually synthesized, then joined (e.g., by enzymatic or chemical ligation methods or polymerase mediated methods) to form any desired continuous sequence. For example, polynucleotides and oligonucleotides of the invention can be prepared by chemical synthesis (e.g., using the classical phosphoramidite method described by Beaucage et al., Tet. Lett., 22:1859-69 [1981], or the method described by Matthes et al., EMBO J., 3:801-05 [1984], as it is typically practiced in automated synthetic methods). According to the phosphoramidite method, oligonucleotides are synthesized (e.g., in an automatic DNA synthesizer), purified, annealed, ligated and cloned in appropriate vectors.

In embodiments, the polynucleotide encodes decarboxylase polypeptide comprising an amino acid sequence selected from the group consisting of SEQ ID NO: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 156, 157, 158, functional fragments thereof, or variants thereof.

An isolated polynucleotide encoding a decarboxylase polypeptide may be manipulated in a variety of ways to provide for expression of the polypeptide. Manipulation of the isolated polynucleotide prior to its insertion into a vector may be desirable or necessary depending on the expression vector. The techniques for modifying polynucleotides and nucleic acid sequences utilizing recombinant DNA methods are well known in the art.

For bacterial host cells, suitable promoters for directing transcription of the nucleic acid constructs of the present invention, include the promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene (See, e.g., Villa-Kamaroff et al., Proc. Natl. Acad. Sci. USA 75: 3727-3731 [1978]), as well as the tac promoter (See, e.g., DeBoer et al., Proc. Natl Acad. Sci. USA 80: 21-25 [1983]). Additional suitable promoters are known to those in the art.

For filamentous fungal host cells, suitable promoters for directing the transcription of the nucleic acid constructs of the present invention include promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (WO 96/00787), as well as the NA2-tpi promoter (a hybrid of the promoters from the genes for Aspergillus niger neutral alpha-amylase and Aspergillus oryzae triose phosphate isomerase), and mutant, truncated, and hybrid promoters thereof.

In a yeast host, useful promoters include, but are not limited to those from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH2/GAP), and Saccharomyces cerevisiae 3-phosphoglycerate kinase, as well as other useful promoters for yeast host cells (See, e.g., Romanos, et al., Yeast 8:423-488 [1992]).

A transcription terminator sequence, a sequence recognized by a host cell to terminate transcription, can be operably linked to the 3′ terminus of the nucleic acid sequence encoding the polypeptide. Any terminator that is functional in the host cell of choice may be used in the present invention. For example, exemplary transcription terminators for filamentous fungal host cells can be obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease. Exemplary terminators for yeast host cells can be obtained from the genes for Saccharomyces cerevisiae enolase, Saccharomyces cerevisiae cytochrome C (CYC1), and Saccharomyces cerevisiae glyceraldehyde-3-phosphate dehydrogenase, as well as other useful terminators for yeast host cells known in the art (See, e.g., Romanos et al., supra).

A leader sequence, a nontranslated region of an mRNA that is important for translation by the host cell, can be operably linked to the 5′ terminus of the nucleic acid sequence encoding the polypeptide. Any leader sequence that is functional in the host cell of choice may be used. Exemplary leaders for filamentous fungal host cells are obtained from the genes for Aspergillus oryzae TAKA amylase and Aspergillus nidulans triose phosphate isomerase. Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

Any polyadenylation sequence which is functional in the host cell of choice may be used in the present invention. A polyadenylation sequence is a sequence operably linked to the 3′ terminus of the nucleic acid sequence and which, when transcribed, is recognized by the host cell as a signal to add polyadenosine residues to transcribed mRNA. Exemplary polyadenylation sequences for filamentous fungal host cells can be from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase, as well as additional useful polyadenylation sequences for yeast host cells known in the art (See, e.g., Guo et al., Mol. Cell. Biol., 15:5983-5990 [1995]).

The 5′ end of the coding sequence of the nucleic acid sequence may inherently contain a signal peptide coding region naturally linked in translation reading frame with the segment of the coding region that encodes the secreted polypeptide. A signal peptide coding region encodes for an amino acid sequence linked to the amino terminus of a polypeptide and directs the encoded polypeptide into the cell's secretory pathway. Alternatively, the 5′ end of the coding sequence may contain a signal peptide coding region that is foreign to the coding sequence. The foreign signal peptide coding region may be required where the coding sequence does not naturally contain a signal peptide coding region. Alternatively, the foreign signal peptide coding region may simply replace the natural signal peptide coding region in order to enhance secretion of the polypeptide. However, any signal peptide coding region which directs the expressed polypeptide into the secretory pathway of a host cell of choice may be used in the present invention.

Effective signal peptide coding regions for bacterial host cells are the signal peptide coding regions obtained from the genes for Bacillus NCIB 11837 maltogenic amylase, Bacillus stearothermophilus alpha-amylase, Bacillus licheniformis subtilisin, Bacillus licheniformis beta-lactamase, Bacillus stearothermophilus neutral proteases (nprT, nprS, nprM), and Bacillus subtilis prsA, as well as additional signal peptides known in the art (See, e.g., Simonen et al., Microbiol. Rev., 57: 109-137 [1993]). Effective signal peptide coding regions for filamentous fungal host cells include, but are not limited to the signal peptide coding regions obtained from the genes for Aspergillus oryzae TAKA amylase, Aspergillus niger neutral amylase, Aspergillus niger glucoamylase, Rhizomucor miehei aspartic proteinase, Humicola insolens cellulase, and Humicola lanuginosa lipase. Useful signal peptides for yeast host cells can be from the genes for Saccharomyces cerevisiae alpha-factor and Saccharomyces cerevisiae invertase, as well as additional useful signal peptide coding regions (See, e.g., Romanos et al., 1992, supra).

A propeptide coding region encodes for an amino acid sequence positioned at the amino terminus of a polypeptide. The resultant polypeptide is known as a proenzyme or propolypeptide (or a zymogen in some cases). A propolypeptide is generally inactive and can be converted to a mature active polypeptide by catalytic or autocatalytic cleavage of the propeptide from the propolypeptide. The propeptide coding region may be obtained from the genes for Bacillus subtilis alkaline protease (aprE), Bacillus subtilis neutral protease (nprT), Saccharomyces cerevisiae alpha-factor, Rhizomucor miehei aspartic proteinase, and Myceliophthora thermophila lactase (WO 95/33836).

Where both signal peptide and propeptide regions are present at the amino terminus of a polypeptide, the propeptide region is positioned next to the amino terminus of a polypeptide and the signal peptide region is positioned next to the amino terminus of the propeptide region.

It may also be desirable to add regulatory sequences, which allow the regulation of the expression of the polypeptide relative to the growth of the host cell. Examples of regulatory systems are those which cause the expression of the gene to be turned on or off in response to a chemical or physical stimulus, including the presence of a regulatory compound. In prokaryotic host cells, suitable regulatory sequences include the lac, tac, and trp operator systems. In yeast host cells, suitable regulatory systems include, as examples, the ADH2 system or GAL1 system. In filamentous fungi, suitable regulatory sequences include the TAKA alpha-amylase promoter, Aspergillus niger glucoamylase promoter, and Aspergillus oryzae gluco amylase promoter.

Other examples of regulatory sequences are those which allow for gene amplification. In eukaryotic systems, these include the dihydrofolate reductase gene, which is amplified in the presence of methotrexate, and the metallothionein genes, which are amplified with heavy metals. In these cases, the nucleic acid sequence encoding the decarboxylase polypeptide of the present invention would be operably linked with the regulatory sequence.

In embodiments, the present invention may also be directed to a recombinant expression vector comprising a polynucleotide encoding a decarboxylase polypeptide or a variant thereof, and one or more expression regulating regions such as a promoter and a terminator, a replication origin, etc., depending on the type of hosts into which they are to be introduced. The various nucleic acid and control sequences described above may be joined together to produce a recombinant expression vector, which may include one or more convenient restriction sites to allow for insertion or substitution of the nucleic acid sequence encoding the polypeptide at such sites. Alternatively, the nucleic acid sequence of the present invention may be expressed by inserting the nucleic acid sequence or a nucleic acid construct comprising the sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus), which can be conveniently subjected to recombinant DNA procedures and can bring about the expression of the polynucleotide sequence. The choice of the vector will typically depend on the compatibility of the vector with the host cell into which the vector is to be introduced. The vectors may be linear or closed circular plasmids. The expression vector may be an autonomously replicating vector (i.e., a vector that exists as an extrachromosomal entity), the replication of which is independent of chromosomal replication, (e.g., a plasmid, an extrachromosomal element, a minichromosome, or an artificial chromosome). The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used.

The expression vector of the present invention preferably contains one or more selectable markers, which permit easy selection of transformed cells. A selectable marker can be a gene the product of which provides for biocide or viral resistance, resistance to heavy metals, prototrophy to auxotrophs, and the like. Examples of bacterial selectable markers are the dal genes from Bacillus subtilis or Bacillus licheniformis, or markers, which confer antibiotic resistance such as ampicillin, kanamycin, chloramphenicol, or tetracycline resistance. Suitable markers for yeast host cells are ADE2, HIS3, LEU2, LYS2, MET3, TRP1, and URA3.

Selectable markers for use in a filamentous fungal host cell include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Embodiments for use in an Aspergillus cell include the amdS and pyrG genes of Aspergillus nidulans or Aspergillus oryzae and the bar gene of Streptomyces hygroscopicus.

The expression vectors of the present invention can contain one or more element(s) that permit integration of the vector into the host cell's genome or autonomous replication of the vector in the cell independent of the genome. For integration into the host cell genome, the vector may rely on the nucleic acid sequence encoding the polypeptide or any other element of the vector for integration of the vector into the genome by homologous or nonhomologous recombination.

Alternatively, the expression vector may contain additional nucleic acid sequences for directing integration by homologous recombination into the genome of the host cell. The additional nucleic acid sequences enable the vector to be integrated into the host cell genome at a precise location(s) in the chromosome(s). To increase the likelihood of integration at a precise location, the integrational elements should preferably contain a sufficient number of nucleic acids, such as 100 to 10,000 base pairs, preferably 400 to 10,000 base pairs, and most preferably 800 to 10,000 base pairs, which are highly homologous with the corresponding target sequence to enhance the probability of homologous recombination. The integrational elements may be any sequence that is homologous with the target sequence in the genome of the host cell. Furthermore, the integrational elements may be non-encoding or encoding nucleic acid sequences. On the other hand, the vector may be integrated into the genome of the host cell by non-homologous recombination.

For autonomous replication, the vector may further comprise an origin of replication enabling the vector to replicate autonomously in the host cell in question. Non-limiting examples of bacterial origins of replication are P15A ori or the origins of replication of plasmids pBR322, pUC19, pACYC177 (which plasmid has the P15A ori), or pACYC184 permitting replication in E. coli, and pUB110, pE194, or pTA1060, permitting replication in Bacillus. Examples of origins of replication for use in a yeast host cell are the 2 micron origin of replication, ARS1, ARS4, the combination of ARS 1 and CEN3, and the combination of ARS4 and CEN6. The origin of replication may be one having a mutation which makes it's functioning temperature-sensitive in the host cell (See, e.g., Ehrlich, Proc. Natl. Acad. Sci. USA 75:1433 [1978]).

More than one copy of a nucleic acid sequence of the present invention may be inserted into a host cell to increase production of the gene product. An increase in the copy number of the nucleic acid sequence can be obtained by integrating at least one additional copy of the sequence into the host cell genome or by including an amplifiable selectable marker gene with the nucleic acid sequence where cells containing amplified copies of the selectable marker gene, and thereby additional copies of the nucleic acid sequence, can be selected for by cultivating the cells in the presence of the appropriate selectable agent.

Many of the expression vectors for use in the present invention are commercially available. Suitable commercial expression vectors include, but are not limited to, p3xFLAG™ expression vectors (Sigma-Aldrich), which include a CMV promoter and hGH polyadenylation site for expression in mammalian host cells and a pBR322 origin of replication and ampicillin resistance markers for amplification in E. coli. Other commercially available suitable expression vectors include but are not limited to the pBluescriptII SK(−) and pBK-CMV vectors (Stratagene), and plasmids derived from pBR322 (Gibco BRL), pUC (Gibco BRL), pREP4, pCEP4 (Invitrogen) or pPoly (See, Lathe et al., Gene 57:193-201 [1987]).

The skilled person will appreciate that, upon production of an enzyme, in particular, depending upon the cell line used and the particular amino acid sequence of the enzyme, post-translational modifications may occur. For example, such post-translational modifications may include the cleavage of certain leader sequences, the addition of various sugar moieties in various glycosylation and phosphorylation patterns, deamidation, oxidation, disulfide bond scrambling, isomerisation, C-terminal lysine clipping, and N-terminal glutamine cyclisation. The present invention encompasses the use of decarboxylase enzymes that have been subjected to, or have undergone, one or more post-translational modifications. Thus, the decarboxylases of the invention include one which has undergone a post-translational modification, such as described herein.

Deamidation is an enzymatic reaction primarily converting asparagine (N) to iso-aspartic acid (iso-aspartate) and aspartic acid (aspartate) (D) at approximately 3:1 ratio. This deamidation reaction is, therefore, related to isomerization of aspartate (D) to iso-aspartate. The deamidation of asparagine and the isomerisation of aspartate, both involve the intermediate succinimide. To a much lesser degree, deamidation can occur with glutamine residues in a similar manner. Oxidation can occur during production and storage (i.e., in the presence of oxidizing conditions) and results in a covalent modification of a protein, induced either directly by reactive oxygen species, or indirectly by reaction with secondary by-products of oxidative stress. Oxidation happens primarily with methionine residues, but may occur at tryptophan and free cysteine residues. Disulfide bond scrambling can occur during production and basic storage conditions. Under certain circumstances, disulfide bonds can break or form incorrectly, resulting in unpaired cysteine residues (—SH). These free (unpaired) sulfhydryls (—SH) can promote shuffling. N-terminal glutamine (Q) and glutamate (glutamic acid) (E) in the decarboxylases are likely to form pyroglutamate (pGlu) via cyclization. Most pGlu formation happens in manufacturing, but it can be formed non-enzymatically, depending upon pH and temperature of processing and storage conditions. C-terminal lysine clipping is an enzymatic reaction catalyzed by carboxypeptidases and is commonly observed in enzymes. Variants of this process include removal of lysine from the enzymes from the recombinant host cell. In the present invention, the post-translational modifications and changes in primary amino acid sequence described above do not result in significant changes in the activity of the decarboxylase enzymes.

Host Cells for Expression of Decarboxylase Polypeptides

In another aspect, the present invention provides a host cell comprising a polynucleotide encoding a decarboxylase polypeptide of the present invention, the polynucleotide being operatively linked to one or more control sequences for expression of the decarboxylase enzyme in the host cell. Host cells for use in expressing the decarboxylase polypeptides encoded by the expression vectors of the present invention are well known in the art and include but are not limited to bacterial cells, (e.g. E. coli, Geobacillus stearothermophilus, Pseudomonas aeruginosa, Lactobacillus kefir, Lactobacillus brevis, Lactobacillus minor, Mycobacterium tuberculosis, Streptomyces coelicolor and Salmonella typhimurium), fungal cells (e.g. Trichoderma reesei and Aspergillus niger), yeast cells (e.g., Saccharomyces cerevisiae, Kluyveromyces lactis or Pichia pastoris), insect cells (e.g. Drosophila S2 and Spodoptera Sf9), animal cells (e.g. CHO, COS, BHK, 293, and Bowes melanoma cells), and plant cells (e.g. Nicotiana genus and Zea mays). Appropriate culture media and growth conditions for the above-described host cells are well known in the art.

Host cells of the present invention may also include, for example, host cells that produce excess quantities of free fatty acids. Host cells that produce excess quantities of free fatty acids may be modified to produce excess quantities of free fatty acids as compared to a corresponding unmodified host cell. The modification may be, for example, genetic modification. Where the modification is a genetic modification, a corresponding unmodified host cell may be, for example, a host cell that lacks the same genetic modification facilitating the production of excess quantities of free fatty acids in the modified host cell. Host cells that produce excess quantities of free fatty acids, as well as methods of making such host cells, are known in the art. In embodiments, beta-oxidation may be eliminated in the host cell, which leads to reduced utilization of fatty acids. Elimination of beta-oxidation in a host cell such as, for example, E. coli, may be accomplished via a ΔfadD deletion, or deletion of a homolog of fadD. In embodiments, the host cell is engineered to encourage production of fatty acids from precursors. This may be accomplished, for example, by the overexpression of one or more thioesterases such as, for example, TesA′ and FatB1, from Cinnamomum camphorum. In embodiments, the host cell is engineered to encourage production of malonyl-coA, which is involved in elongating fatty acid chains. This may be accomplished, for example, by the overexpression of an acetyl-coA carboxylase (ACC) such as, for example, the acetyl-coA carboxylase (ACC) from E. coli. In embodiments, the host cell is engineered to limit the fatty acid yield to shorter chain fatty acids in the C12-C14 range. This may be accomplished, for example, by the overexpression of the thioesterase from Umbellularia californica (UcTE) (Lennen et al., Trends in Cell Biology 30:12, pp. 659-667, 2012). In embodiments, the host cell is engineered for reverse beta-oxidation. Host cells such as, for example, E. coli, may be engineered for reverse beta-oxidation by, for example, reducing or eliminating the activity of the fadR, atoC(c), crp, arcA, adhE, pta, frdA, fucO, yqhD, and fadD genes or homologs thereof, as well as overexpressing FadBA and at least one thioesterase from the group including TesA TesB, FadM, and YciA, or homologs thereof. The particular thioesterase overexpressed may impact the chain length distribution of the final products (Dellomonaco et al., Nature 475, pp. 355-359, 2011). In embodiments, host cells of the present disclosure may overexpress a FatB2 protein from Umbellularia californica, which may be codon-optomized.

Polynucleotides for expression of the decarboxylase may be introduced into cells by various methods known in the art. Techniques include among others, electroporation, biolistic particle bombardment, liposome mediated transfection, calcium chloride transfection, and protoplast fusion. Various methods for introducing polynucleotides into cells will be apparent to the skilled artisan.

Methods of Producing Decarboxylase Polypeptides

Standard methods of culturing organisms such as, for example, bacteria and yeast, for production of enzymes are well-known in the art and are described herein. For example, host cells may be cultured in a standard growth media under standard temperature and pressure conditions, and in an aerobic environment. Standard growth media for various host cells are commercially available and well-known in the art, as are standard conditions for growing various host cells.

Decarboxylase enzymes expressed in a host cell can be recovered from the cells and or the culture medium using any one or more of the well-known techniques for protein purification, including, among others, lysozyme treatment, sonication, filtration, salting-out, ultra-centrifugation, and chromatography. Suitable solutions for lysing and the high efficiency extraction of proteins from bacteria, such as E. coli, are commercially available under the trade name CelLytic B (Sigma-Aldrich). Chromatographic techniques for isolation of the decarboxylase polypeptide include, among others, reverse phase chromatography high performance liquid chromatography (HPLC), ion exchange chromatography, gel electrophoresis, and affinity chromatography. Conditions for purifying a particular enzyme will depend, in part, on factors such as net charge, hydrophobicity, hydrophilicity, molecular weight, molecular shape, etc., and will be apparent to those having skill in the art.

The decarboxylases may also be prepared and used in the form of cells expressing the enzymes, as crude extracts, or as isolated or purified preparations. The decarboxylases may be prepared as lyophilizates, in powder form (e.g., acetone powders), or prepared as enzyme solutions. In embodiments, the decarboxylases can be in the form of substantially pure preparations.

Consumer Product Compositions

In certain embodiments, the present invention relates to consumer product compositions comprising a surfactant and a P450 fatty acid decarboxylase. The consumer product compositions, when used to contact soiled surfaces having disposed thereon soils comprising fatty acid, can convert the fatty acid of the soil into an enzymatic product, such as a terminal olefin. In this regard, the consumer product compositions of the present invention can exhibit improved cleaning performance, or equivalent cleaning performance while utilizing lower levels of surfactant in the consumer product composition. Preferred fatty acids are stearic acid, oleic acid, linoleic acid, and linolenic acid.

A consumer product composition of the present invention may be a manual dishwashing composition, preferably in liquid form. It typically contains from 30% to 95%, preferably from 40% to 90%, more preferably from 50% to 85% by weight of the composition of a liquid carrier in which the other essential and optional components are dissolved, dispersed or suspended. One preferred component of the liquid carrier is water.

The pH of a consumer product composition of the present invention, measured as a 10% product concentration in demineralized water at 20° C., may be adjusted to between 3 and 14, more preferably between 4 and 13, more preferably between 6 and 12 and most preferably between 8 and 10. The pH of the consumer product composition can be adjusted using pH modifying ingredients known in the art.

The consumer product composition herein may optionally comprise a number of other consumer product adjunct ingredients such as enzyme stabilizers, surfactants, co-enzymes, a source of hydrogen peroxide, salts, hydrotropes, chelants, builders, dispersants, dye transfer inhibitors, bleach, stabilizers/thickeners, perfume, conditioning agents, hueing agents, structurants, solvents, aqueous carrier, and mixtures thereof. Consumer product adjunct ingredients also include scrubbing particles, malodor control agents, pigments, dyes, opacifiers, pH adjusters and buffering means (e.g., carboxylic acids such as citric acid, HCl, NaOH, KOH, alkanolamines, phosphoric and sulfonic acids, carbonates such as sodium carbonates, bicarbonates, sesquicarbonates, borates, silicates, phosphates, imidazole and alike).

Enzyme Stabilizers

The composition of the present invention may comprise an enzyme stabilizer, selected from the group consisting of chemical and physical stabilizers, preferably the physical stabilizer comprises encapsulating the enzyme. Suitable enzyme stabilizers may be selected from the group consisting of (a) univalent, bivalent and/or trivalent cations preferably selected from the group of inorganic or organic salts of alkaline earth metals, alkali metals, aluminum, iron, copper and zinc, preferably alkali metals and alkaline earth metals, preferably alkali metal and alkaline earth metal salts with halides, sulfates, sulfites, carbonates, hydrogencarbonates, nitrates, nitrites, phosphates, formates, acetates, propionates, citrates, maleates, tartrates, succinates, oxalates, lactates, and mixtures thereof. In a preferred embodiment the salt is selected from the group consisting of sodium chloride, calcium chloride, potassium chloride, sodium sulfate, potassium sulfate, sodium acetate, potassium acetate, sodium formate, potassium formate, calcium lactate, calcium nitrate and mixtures thereof. Most preferred are salts selected from the group consisting of calcium chloride, potassium chloride, potassium sulfate, sodium acetate, potassium acetate, sodium formate, potassium formate, calcium lactate, calcium nitrate, and mixtures thereof, and in particular potassium salts selected from the group of potassium chloride, potassium sulfate, potassium acetate, potassium formate, potassium propionate, potassium lactate and mixtures thereof. Most preferred are potassium acetate and potassium chloride. Preferred calcium salts are calcium formate, calcium lactate and calcium nitrate including calcium nitrate tetrahydrate. Calcium and sodium formate salts may be preferred. These cations are present at at least about 0.01 wt %, preferably at least about 0.03 wt %, more preferably at least about 0.05 wt %, most preferably at least about 0.25 wt % up to 2 wt % or even up to 1 wt % by weight of the total composition. These salts are formulated from 0.1 wt % to 5 wt %, preferably from 0.2 wt % to 4 wt %, more preferably from 0.3 wt % to 3 wt %, most preferably from 0.5 wt % to 2 wt % relative to the total weight of the composition. Further enzyme stabilizers can be selected from the group (b) carbohydrates selected from the group consisting of oligosaccharides, polysaccharides and mixtures thereof, such as a monosaccharide glycerate as described in WO201219844; (c) mass efficient reversible protease inhibitors selected from the group consisting of phenyl boronic acid and derivatives thereof, preferably 4-formyl phenylboronic acid; (d) alcohols such as 1,2-propane diol, propylene glycol; (e) peptide aldehyde stabilizers such as tripeptide aldehydes such as Cbz-Gly-Ala-Tyr-H, or disubstituted alaninamide; (f) carboxylic acids such as phenyl alkyl dicarboxylic acid as described in WO2012/19849 or multiply substituted benzyl carboxylic acid comprising a carboxyl group on at least two carbon atoms of the benzyl radical such as described in WO2012/19848, phthaloyl glutamine acid, phthaloyl asparagine acid, aminophthalic acid and/or an oligoamino-biphenyl-oligocarboxylic acid; and (g) mixtures thereof.

Antioxidants

Antioxidant compounds and free radical scavengers can generally protect enzyme from degradation by preventing excessive generation of singlet oxygen and peroxy radicals that promote alteration of enzyme structure leading to short TON of Enzymes. Not to be limited by theory, a general discussion of the mode of action for antioxidants and free radical scavengers is disclosed in Kirk Othmer, The Encyclopedia of Chemical Technology, Volume 3, pages 128-148, Third Edition (1978).

The composition may optionally contain an anti-oxidant present from about 0.001 to about 2% by weight. Preferably the antioxidant is present at a concentration in the range 0.01 to 0.1% by weight. Mixtures of anti-oxidants may be used and in some embodiments, may be preferred. One or more antioxidants may be incorporated the composition.

One class of anti-oxidants used in the present invention is alkylated phenols, having the general formula:

[chemical structure]

wherein R is C1-C22 linear or branched alkyl, preferably methyl or branched C3-C6 alkyl, Ci-C6 alkoxy, preferably methoxy, or CH2CH2C(0)0R′, wherein R′ is H, a charge balancing counterion or C1-C22 linear or branched alkyl; Ri is a C3-C6 branched alkyl, preferably tert-butyl; x is 1 or 2. Hindered phenolic compounds are a preferred type of alkylated phenols having this formula. A preferred hindered phenolic compound of this type is 3,5-di-tert-butyl-4-hydroxytoluene (BHT). Furthermore, the anti-oxidant used in the composition may be selected from the group consisting of a-, b-, g-, d-tocopherol, ethoxyquin, 2,2 4-trimethyl-1,2-dihydroquinoline, 2,6-di-tert-butyl hydroquinone, tert-butyl hydroxyanisole, lignosulphonic acid and salts thereof, and mixtures thereof. It is noted that ethoxyquin (1,2-dihydro-6-ethoxy-2,2,4-trimethylquinoline) is marketed under the name Raluquin™ by the company Raschig™. Other types of anti-oxidants that may be used in the composition are 6-hydroxy-2,5,7,8-tetramethylchroman-2-carboxylic acid (Trolox™) and 1,2-benzisothiazoline-3-one (Proxel GXL™)

A further class of anti-oxidants which may be suitable for use in the composition is a benzOkofuran or benzopyran derivative having the formula:

[chemical structure]

wherein Ri and R2 are each independently alkyl or Ri and R2 can be taken together to form a C5-C6 cyclic hydrocarbyl moiety; B is absent or CH2; R4 is Ci-Ce alkyl; R5 is hydrogen or —C(0)R3 wherein R3 is hydrogen or C1-C19 alkyl; Re is Ci-Ce alkyl; R7 is hydrogen or Ci-Ce alkyl; X is —CH2OH, or —CH2A wherein A is a nitrogen comprising unit, phenyl, or substituted phenyl. Preferred nitrogen comprising A units include amino, pyrrolidino, piperidino, morpholino, piperazino, and mixtures thereof.

Anti-oxidants such as tocopherol sorbate, butylated hydroxyl benxoic acids and their salts, gallic acid and its alkyl esters, ascorbic, citric, tartric, uric acid and its salts, sorbic acid and its salts, and dihydroxyfumaric acid and its salts may also be used. In one aspect, the most preferred types of anti-oxidant for use in the composition are 3,5-di-tert-butyl-4-hydroxytoluene (BHT), a-, b-, g-, d-tocopherol, 1,2-benzisothiazoline-3-one (Proxel GXL™), anthocyanins, carotene, catechins, flavonoids, lutein, lycopene and mixtures thereof. In another aspect, the most preferred types of anti-oxidant for use in the composition are hindered phenols, diarylamines (including phenoxazines with a maximum molar extinction coefficient in the wavelength range from 400 to 750 nm of less than 1,000 M_1ah_1), and mixtures thereof. In preferred mixtures, the number of equivalents of hindered phenol initially formulated will normally be greater than or equal to the number of equivalents of diarylamine.

Surfactants

The consumer product compositions of the present invention may comprise greater than about 0.1% by weight of a surfactant or mixture of surfactants. Surfactant levels cited herein are on a 100% active basis, even though common raw materials such as sodium lauryl sulphate may be supplied as aqueous solutions of lower activity. In embodiments of the present invention, a consumer product composition may include surfactant in an amount of from about 1 wt % to about 60 wt %, from about 5 wt % to about 50 wt %, by weight of the consumer product composition.

Suitable surfactants for use herein include anionic surfactants, amphoteric surfactants, nonionic surfactants, zwitterionic surfactants, cationic surfactants, and mixtures thereof. In embodiments, the consumer product composition comprises one or more anionic surfactants and one or more co-surfactants selected from the group consisting of amphoteric surfactant, zwitterionic surfactant, and mixtures thereof.

Useful anionic surfactants herein include the water-soluble salts of alkyl sulphates and alkyl ether sulphates having from 10 to 18 carbon atoms in the alkyl radical and the water-soluble salts of sulphonated monoglycerides of fatty acids having from 10 to 18 carbon atoms. Sodium lauryl sulphate and sodium coconut monoglyceride sulphonates are examples of anionic surfactants of this type.

Suitable cationic surfactants useful in the present invention can be broadly defined as derivatives of aliphatic quaternary ammonium compounds having one long alkyl chain containing from about 8 to 18 carbon atoms such as lauryl trimethylammonium chloride; cetyl pyridinium chloride; benzalkonium chloride; cetyl trimethylammonium bromide; di-isobutylphenoxyethyl-dimethylbenzylammonium chloride; coconut alkyltrimethyl-ammonium nitrite; cetyl pyridinium fluoride; etc. Certain cationic surfactants can also act as germicides in the compositions disclosed herein.

Suitable nonionic surfactants that can be used in the compositions of the present invention can be broadly defined as compounds produced by the condensation of alkylene oxide groups (hydrophilic in nature) with an organic hydrophobic compound which may be aliphatic and/or aromatic in nature. Examples of suitable nonionic surfactants include the poloxamers; sorbitan derivatives, such as sorbitan di-isostearate; ethylene oxide condensates of hydrogenated castor oil, such as PEG-30 hydrogenated castor oil; ethylene oxide condensates of aliphatic alcohols or alkyl phenols; products derived from the condensation of ethylene oxide with the reaction product of propylene oxide and ethylene diamine; long chain tertiary amine oxides; long chain tertiary phosphine oxides; long chain dialkyl sulphoxides and mixtures of such materials. These materials are useful for stabilising foams without contributing to excess viscosity build for the consumer product composition.

Zwitterionic surfactants can be broadly described as derivatives of aliphatic quaternary ammonium, phosphonium, and sulphonium compounds, in which the aliphatic radicals can be straight chain or branched, and wherein one of the aliphatic substituents contains from about 8 to 18 carbon atoms and one contains an anionic water-solubilising group, e.g., carboxy, sulphonate, sulphate, phosphate or phosphonate.

Surfactants can provide a desirable foaming quality. Suitable surfactants are those which are reasonably stable and foam throughout a wide pH range. The surfactant may be anionic, nonionic, amphoteric, zwitterionic, cationic, or mixtures thereof. Anionic surfactants useful herein include the water-soluble salts of alkyl sulfates having from 8 to 20 carbon atoms in the alkyl radical (e.g., sodium alkyl sulfate) and the water-soluble salts of sulfonated monoglycerides of fatty acids having from 8 to 20 carbon atoms. Sodium lauryl sulfate and sodium coconut monoglyceride sulfonates are examples of anionic surfactants of this type. Other suitable anionic surfactants are sarcosinates, such as sodium lauroyl sarcosinate, taurates, sodium lauryl sulfoacetate, sodium lauroyl isethionate, sodium laureth carboxylate, and sodium dodecyl benzenesulfonate. Mixtures of anionic surfactants can also be employed. Many suitable anionic surfactants are disclosed by Agricola et al., U.S. Pat. No. 3,959,458, issued May 25, 1976, incorporated herein in its entirety by reference. Nonionic surfactants which can be used in the compositions of the present invention can be broadly defined as compounds produced by the condensation of alkylene oxide groups (hydrophilic in nature) with an organic hydrophobic compound which may be aliphatic or alkyl-aromatic in nature. Examples of suitable nonionic surfactants include poloxamers (sold under trade name Pluronic), polyoxyethylene, polyoxyethylene sorbitan esters (sold under trade name Tweens), fatty alcohol ethoxylates, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides, and mixtures of such materials. The amphoteric surfactants useful in the present invention can be broadly described as derivatives of aliphatic secondary and tertiary amines in which the aliphatic radical can be a straight chain or branched and wherein one of the aliphatic substituents contains from about 8 to about 18 carbon atoms and one contains an anionic water-solubilizing group, e.g., carboxylate, sulfonate, sulfate, phosphate, or phosphonate. Other suitable amphoteric surfactants are betaines, specifically cocamidopropyl betaine. Mixtures of amphoteric surfactants can also be employed. Many of these suitable nonionic and amphoteric surfactants are disclosed by Gieske et al., U.S. Pat. No. 4,051,234, issued Sep. 27, 1977, incorporated herein by reference in its entirety. The present composition typically comprises one or more surfactants each at a level of from about 0.1% to about 25%, preferably from about 0.5% to about 8%, and most preferably from about 1% to about 6%, by weight of the composition.

Source of Hydrogen Peroxide

It may be preferred for the composition to comprise a source of hydrogen peroxide. Sources of hydrogen peroxide include, for example, inorganic perhydrate salts, including alkali metal salts such as sodium salts of perborate (usually mono- or tetra-hydrate), percarbonate, persulphate, perphosphate, persilicate salts and mixtures thereof. In one aspect of the invention the inorganic perhydrate salts are selected from the group consisting of sodium salts of perborate, percarbonate and mixtures thereof. In some compositions, percarbonate salts are preferred. When employed, inorganic perhydrate salts are typically present in amounts of from 0.05 to 40 wt %, or 1 to 30 wt % of the overall consumer product composition and are typically incorporated into such compositions as a crystalline solid that may be coated. Suitable coatings include, inorganic salts such as alkali metal silicate, carbonate or borate salts or mixtures thereof, or organic materials such as water-soluble or dispersible polymers, waxes, oils or fatty soaps. These may be present in combination with bleach activators and/or bleach catalysts. In other compositions, hydrogen peroxide is preferred. When employed, hydrogen peroxide is typically present in amounts of from 0.05 to 40 wt %, or 1 to 30 wt % of the overall consumer product composition.

Suitable bleach activators are those having R—(C═O)—L wherein R is an alkyl group, optionally branched, having, when the bleach activator is hydrophobic, from 6 to 14 carbon atoms, or from 8 to 12 carbon atoms and, when the bleach activator is hydrophilic, less than 6 carbon atoms or even less than 4 carbon atoms; and L is leaving group. Examples of suitable leaving groups are benzoic acid and derivatives thereof—especially benzene sulphonate. Suitable bleach activators include dodecanoyl oxybenzene sulphonate, decanoyl oxybenzene sulphonate, decanoyl oxybenzoic acid or salts thereof, 3,5,5-trimethyl hexanoyloxybenzene sulphonate, tetraacetyl ethylene diamine (TAED) and nonanoyloxybenzene sulphonate (NOBS). While any suitable bleach activator may be employed, it may be preferred if the subject composition comprises NOBS, TAED or mixtures thereof.

Suitable bleach catalysts include one or more bleach catalysts capable of accepting an oxygen atom from a peroxyacid and/or salt thereof and transferring the oxygen atom to an oxidizeable substrate. Suitable bleach catalysts include, but are not limited to: iminium cations and polyions; iminium zwitterions; modified amines; modified amine oxides; N-sulphonyl imines; N-phosphonyl imines; N-acyl imines; thiadiazole dioxides; perfluoroimines; cyclic sugar ketones and alpha amino-ketones and mixtures thereof.

Suitable bleach catalysts include oxaziridinium bleach catalysts, transition metal bleach catalysts, especially manganese and iron bleach catalysts. A suitable bleach catalyst has a structure corresponding to general formula below:

[chemical structure]

wherein R¹³ is selected from the group consisting of 2-ethylhexyl, 2-propylheptyl, 2-butyloctyl, 2-pentylnonyl, 2-hexyldecyl, n-dodecyl, n-tetradecyl, n-hexadecyl, n-octadecyl, iso-nonyl, iso-decyl, iso-tridecyl and iso-pentadecyl.

Another suitable source of hydrogen peroxide includes pre-formed peracids. Suitable preformed peracids include, but are not limited to compounds selected from the group consisting of pre-formed peroxyacids or salts thereof typically a percarboxylic acids and salts, percarbonic acids and salts, perimidic acids and salts, peroxymonosulfuric acids and salts, for example, Oxone®, and mixtures thereof. Suitable examples include peroxycarboxylic acids or salts thereof, or peroxysulphonic acids or salts thereof. Typical peroxycarboxylic acid salts suitable for use herein have a chemical structure corresponding to the following chemical formula:

[chemical structure]

wherein: R¹⁴ is selected from alkyl, aralkyl, cycloalkyl, aryl or heterocyclic groups; the R¹⁴ group can be linear or branched, substituted or unsubstituted; having, when the peracid is hydrophobic, from 6 to 14 carbon atoms, or from 8 to 12 carbon atoms and, when the peracid is hydrophilic, less than 6 carbon atoms or even less than 4 carbon atoms and Y is any suitable counter-ion that achieves electric charge neutrality, preferably Y is selected from hydrogen, sodium or potassium. R¹⁴ may be a linear or branched, substituted or unsubstituted C₆₋₉ alkyl. The peroxyacid or salt thereof may be selected from peroxyhexanoic acid, peroxyheptanoic acid, peroxyoctanoic acid, peroxynonanoic acid, peroxydecanoic acid, any salt thereof, or any combination thereof. Peroxyacids that may be used include phthalimido-peroxy-alkanoic acids, in particular ε-phthalimido peroxy hexanoic acid (PAP). The peroxyacid or salt thereof may have a melting point in the range of from 30° C. to 60° C.

The pre-formed peroxyacid or salt thereof can also be a peroxysulphonic acid or salt thereof, typically having a chemical structure corresponding to the following chemical formula:

[chemical structure]

wherein: R¹⁵ is selected from alkyl, aralkyl, cycloalkyl, aryl or heterocyclic groups; the R¹⁵ group can be linear or branched, substituted or unsubstituted; and Z is any suitable counter-ion that achieves electric charge neutrality, preferably Z is selected from hydrogen, sodium or potassium. Preferably R¹⁵ is a linear or branched, substituted or unsubstituted C₄₋₁₄, preferably C₆₋₁₄ alkyl. Preferably such bleach components may be present in the compositions of the invention in an amount from 0.01 to 50%, most preferably from 0.1% to 20%.

Hydrogen peroxide may also be provided by the incorporation of one or more hydrogen peroxide producing enzymes such as alcohol oxidoreductases, aldehyde oxidoreductases, amino acid oxidoreductases, and monoamine oxidases. These enzymes can convert in situ (e.g. in the washing process) substrates such as carbohydrates, proteins, amino acids, alcohols, amines, or other substrates either from a soil or from a material also present in the composition, to generate hydrogen peroxide. Since this will tend to generate low levels of hydrogen peroxide this may be preferred. Non-limiting examples of hydrogen peroxide producing enzymes are: glycolate oxidases (EC 1.1.3.1), L-lactate oxidases (EC 1.1.3.2), malate oxidases (EC 1.1.3.3), glucose oxidases (EC 1.1.3.4), glycerol oxidases (EC 1.1.3.B4), hexose oxidases (EC 1.1.3.5), cholesterol oxidases (EC 1.1.3.6), aryl-alcohol oxidases (EC 1.1.3.7), L-gulonolactone oxidases (EC 1.1.3.8), galactose oxidases (EC 1.1.3.9), pyranose oxidases (EC 1.1.3.10), L-sorbose oxidases (EC 1.1.3.11), alcohol oxidases (EC 1.1.3.13), (S)-2-hydroxy-acid oxidases (EC 1.1.3.15), chlorine oxidases (EC 1.1.3.17), secondary-alcohol oxidases (EC 1.1.3.18), long-chain-alcohol oxidases (EC 1.1.3.20), thiamine oxidases (EC 1.1.3.23), nucleoside oxidases (EC 1.1.3.28, EC 1.1.3.39), polyvinyl-alcohol oxidases (EC 1.1.3.30), vanillyl-alcohol oxidases (EC 1.1.3.38), D-mannitol oxidase ((EC 1.1.3.40), alditol oxidases (EC 1.1.3.41), glucooligosaccharide oxidases (EC 1.1.99.B3), cellobiose dehydrogenase (EC 1.1.99.18), aldehyde oxidases (EC 1.2.3.1), pyruvate oxidases (EC 1.2.3.3), oxalate oxidases (EC 1.2.3.4), glyoxylate oxidases (EC 1.2.3.5), D-aspartate oxidases (EC 1.4.3.1), L-amino acid oxidases (EC 1.4.3.2), D-amino acid oxidases (EC 1.4.3.3), monoamine oxidases (EC 1.4.3.4), D-glutamate oxidases (EC 1.4.3.7), ethanolamine oxidases (EC 1.4.3.8), protein-lysine 6-oxidases (EC 1.4.3.13), L-lysine oxidases (EC 1.4.3.14), D-glutamate (D-aspartate) oxidases (EC 1.4.3.15), L-aspartate oxidases (EC 1.4.3.16), glycine oxidases (EC 1.4.3.19), L-lysine 6-oxidases (EC 1.4.3.20), primary-amine oxidases (EC 1.4.3.21), diamine oxidases (EC 1.4.3.22), L-arginine oxidases (EC 1.4.3.25), non-specific polyamine oxidases (EC 1.5.3.17), other alcohol oxidoreductases (EC 1.1.X.X), other aldehyde oxidoreductases (EC 1.2.X.X), other amino acid oxidoreductases or monoamine oxidases (EC 1.3.X.X), and other amine oxidoreductases (EC 1.5.X.X). The hydrogen peroxide producing enzyme can be fused to the P450 fatty acid decarboxylase to form a single polypeptide or can be independent enzymes.

In some embodiments, the hydrogen peroxide source can be substituted by: a) a source of nicotinamide adenine dinucleotide (NADH) or nicotinamide adenine dinucleotide phosphate (NADPH) and b) an enzymatic redox system. Non-limiting examples of enzymatic redox systems are: the reductase domain of Bacillus megaterium CYP102A1 (P450BM3), the RhFred reductase domain from Rhodococcus sp. NCIMB 9784, the flavodoxin (Fld)/ferrodoxin reductase (FdR, EC 1.18.1.2 and EC 1.18.1.3) redox system, the putidaredoxin (Pd)/putidaredoxin reductase (PdR, EC 1.18.1.5) system, the rubredoxin/rubredoxin reductase (EC 1.18.1.1 and EC 1.18.1.4) system, and the adrenoxin/adrenodoxin reductase (EC 1.18.1.6) system. In some embodiments, the composition may also comprise a dehydrogenase-based NADH or NADPH regeneration system, such as the phosphonate/phosphonate dehydrogenase (EC 1.20.1.1) system.

Method of Using the Consumer Product Composition

The present invention relates to methods of cleaning a surface having disposed thereon a soil comprising fatty acid selected from the group consisting of: stearic acid, oleic acid, linoleic acid, linolenic acid, and mixtures thereof, said method comprising the steps of: (a) contacting said soil disposed on said surface with a consumer product composition comprising a surfactant and a P450 fatty acid decarboxylase; and (b) converting said fatty acid of said soil on said surface into a terminal olefin.

The method can further comprise the step of removing the consumer product composition from the surface, e.g. by rinsing the composition from the surface (e.g. with water) or mechanically removing the composition from the surface (e.g. by wiping composition from the surface).

The method can further include the step of diluting the consumer product composition with water to form a diluted consumer product composition and then contacting the surface with the diluted consumer product composition.

Preferred surfaces treated with the consumer product composition of the present invention include surfaces selected from the group consisting of hair, skin, fabric, dishware, tableware, and household hard surfaces.

The present invention further relates to methods of cleaning a surface including a method of manually washing soiled articles, preferably dishware, comprising the step of: delivering a composition of the invention into a volume of water to form a wash solution and immersing the soiled articles in the wash solution, wherein the soil on the soiled articles comprise at least one fatty acid selected from the group consisting of: stearic acid, oleic acid, linoleic acid, linolenic acid, and mixtures thereof.

The P450 fatty acid decarboxylase may be present at a concentration of from 0.005 ppm to 15 ppm, preferably from 0.01 ppm to 5 ppm, more preferably from 0.02 ppm to 0.5 ppm, in an aqueous wash liquor during the washing process. As such, the composition herein will be applied in its diluted form to the soiled surface. Soiled surfaces e.g. dishes are contacted with an effective amount, typically from 0.5 mL to 20 mL (per 25 dishes being treated), preferably from 3 mL to 10 mL, of the consumer product composition of the present invention, preferably in liquid form, diluted in water. The actual amount of consumer product composition used will be based on the judgment of user, and will typically depend upon factors such as the particular product formulation of the composition, including the concentration of active ingredients in the composition, the number of soiled surfaces to be cleaned, the degree of soiling on the surfaces, and the like.

The present invention also includes the use of P450 fatty acid decarboxylases to provide increased suds longevity in an aqueous wash liquor comprising soil, wherein the soil comprises fatty acid. The enzymes are preferably comprised in a detergent composition, especially a detergent composition of the present invention, which is used for manually washing dishes.

### TEST METHODS

The following assays set forth are used to illustrate certain aspects of the invention described and claimed herein, such that the present invention may be more fully understood.

### Test Method 1

### Glass Vial Suds Mileage Method

The objective of the glass vial suds mileage test method is to measure the evolution of suds volume over time generated by a certain solution of detergent composition in the presence of a greasy soil, e.g., olive oil. The steps of the method are as follows:
1. Test solutions are prepared by subsequently adding aliquots at room temperature of: a) 10 g of an aqueous detergent solution at specified detergent concentration and water hardness, b) 1.0 g of an aqueous protein (or mixture of proteins) solution at specified concentration and water hardness), and c) 0.11 g of olive oil (Bertolli®, Extra Virgin Olive Oil), into a 40 mL glass vial (dimensions: 95 mm H×27.5 mm D). For the reference samples, the protein solutions are substituted with 1.0 mL of demineralized water.
2. The test solutions are mixed in the closed test vials by stirring at room temperature for 2 minutes on a magnetic stirring plate (IKA, model # RTC B S001; VWR magnetic stirrer, catalog # 58949-012; 500 RPM), followed by manually shaking for 20 seconds with an upwards downwards movement (about 2 up and down cycles per second, +/−30 cm up and 30 cm down).
3. Following the shaking, the test solutions in the closed vials are further stirred on a magnetic stirring plate (IKA, model # RTC B S001; VWR magnetic stirrer, catalog # 58949-012; 500 RPM) for 60 minutes inside a water bath at 46° C. to maintain a constant temperature. The samples are then shaken manually for another 20 seconds as described above and the initial suds heights (H1) are recorded with a ruler.
4. The samples are incubated for an additional 30 minutes inside the water bath at 46° C. while stirring (IKA, model # RTC B S001; VWR magnetic stirrer, catalog # 58949-012; 500 RPM), followed by manual shaking for another 20 seconds as described above. The final suds heights (H2) are recorded.
5. Protein solutions that produce larger suds heights (H1 and H2), preferably combined with lower drops in suds height between H1 and H2, are more desirable.

### Test Method 2

### Small Sink Suds Mileage Method

The evolution of the suds volume generated by a solution of a liquid detergent composition can be determined while adding soil loads periodically as follows. An aliquot of 500 mL of solution of the liquid detergent composition in 15 dH hard water (final concentration of 0.12 w %, initial temperature 46° C.) is added into a cylindrical container (dimensions: 150 mm D×150 mm H). The container is incubated in a water bath during the test to maintain the temperature of the solution between 40° C. and 46° C. An initial suds volume is generated in the container by mechanical agitation at 135 rpm for 120 seconds with a paddle (dimensions: 50 mm×25 mm) positioned in the middle of the container.

Then, an aliquot of 0.5 mL of greasy soil (composition: see Table 1, 0.5 mL) is dosed into the solution using a 20-mL syringe and an automated pump (KDS Legato 110 Single Syringe I/W Pump), while the paddle rotates into the solution at 135 rpm for 14 seconds.

TABLE 1
Greasy soil composition.

| Ingredient | Weight % |
|---|---|
| Crisco oil | 12.730 |
| Crisco shortening | 27.752 |
| Lard | 7.638 |
| Refined Rendered Edible Beef Tallow | 51.684 |
| Oleic Acid, 90% (Techn) | 0.139 |
| Palmitic Acid, 99+% | 0.036 |
| Stearic Acid, 99+% | 0.021 |

After mixing, the solution is incubated for 166 additional seconds before the next cycle. The soil injecting, paddling, and incubation steps are repeated every 180 seconds until the end-point is reached and the amount of soil additions needed is recorded. The end-point occurs when a clear suds-free ring that circles the impeller at least half way around is observed two or more consecutive times. The complete process is repeated a number of times and the average of the number of additions for all the replicates is calculated for each liquid detergent composition.

Finally, the suds mileage index is then calculated as: (average number of soil additions for test liquid detergent composition)/(average number of soil additions for reference liquid detergent composition)×100. Depending on the test purpose the skilled person could choose to select an alternative water hardness, solution temperature, product concentration or soil type.

### EXAMPLES

The following examples are provided to further illustrate the present invention and are not to be construed as limitations of the present invention, as many variations of the present invention are possible without departing from its spirit or scope.

### Example 1

### Production of Micrococcus lylae OleTML

Micrococcus lylae OleTML (SEQ ID NO: 2) is a P450 fatty acid decarboxylase that converts medium chain fatty acids (e.g. palmitic acid) into the corresponding terminal olefins and that is included as an example of the current invention. A codon optimized gene (SEQ ID NO: 154) encoding for a Micrococcus lylae OleTML variant, including an N-terminal amino acid sequence containing a His-tag and a TEV protease cleavage site (SEQ ID NO: 155), was designed and synthesized. After gene synthesis, the protein was expressed and purified by Genscript (Piscataway, N.J.). In brief, the complete synthetic gene sequence was subcloned into a pET30a vector for heterologous expression. Escherichia coli C41 (DE3) cells were co-transformed with the recombinant plasmid and with plasmid pTf16. A single colony was inoculated into TB medium containing kanamycin and chloramphenicol. Cultures were incubated at 15° C. for 16 h at 200 rpm and L-arabinose (final concentration 0.1%), δ-aminolevulinic acid (final concentration 0.25 mM) and isopropyl β-D-1-thiogalactopyranoside (IPTG, final concentration 1 mM) were added to induce protein expression. Cells were harvested by centrifugation at 5000 rpm and 4° C. and the pellets were lysed by sonication. After centrifugation, the supernatant was collected and the protein was purified by one-step purification using a nickel affinity column and standard protocols known in the art. The protein was stored in a buffer containing 50 mM Tris-HCl, 500 mM NaCl, and 10% Glycerol at pH 8.0. The final protein concentration was 0.93 mg/mL as determined by Bradford protein assay with BSA as a standard (ThermoFisher, catalog # 23236).

### Example 2

### Production of Macrococcus bovicus OleTMB

Macrococcus bovicus OleTMB (SEQ ID NO: 22) is a predicted P450 fatty acid decarboxylase that converts medium chain fatty acids (e.g. linoleic acid) into the corresponding terminal olefins and that is included as an example of the current invention. A codon optimized gene (SEQ ID NO: 159) encoding for an OleTMB decarboxylase variant, including an N-terminal amino acid sequence containing a His-tag and a TEV protease cleavage site was designed and synthesized by Genscript. After gene synthesis, the protein was expressed and purified. In brief, the complete synthetic gene sequence was subcloned into a pET30a using the NdeI/XhoI cloning sites. For heterologous expression, Escherichia coli BL21 (DE3) cells were transformed with the recombinant plasmid and a single colony was inoculated into LB medium containing kanamycin (50 mg/L). Pre-starter cultures were then inoculated into a fermentor containing Magic Media (ThermoFisher, Catalog # K6803) supplemented with kanamycin (50 mg/L) and incubated at 16° C. for 72 h. At an OD₆₀₀ₙₘ=0.5-1.0, 5-aminolevulinic acid (final concentration 0.5 mM) was added. Cells were harvested by centrifugation at 5000 rpm and 4° C. and the pellets were lysed using a bacterial cell lysis buffer (B-PER—ThermoFisher, Waltham, Mass.). After centrifugation, the supernatant was collected, and the protein was purified by one-step purification using a HisPur™ Ni-NTA Spin Columns (Thermo Scientific, Catalog # 88226) and standard protocols known in the art. The protein was concentrated using a 10 kDa MW cutoff Amicon Ultra Centrifugal Filter Unit (MilliporeSigm, Catalog# UFC901024), followed by desalting using a disposable PD-10 desalting column (GE Healthcare Life Sciences, Catalog# 17085101) and a buffer containing 50 mM Tris-HCl, 500 mM NaCl, and 10% Glycerol at pH 8.0. The purified enzyme was stored at −80° C. until use.

### Example 3

### Production of Staphylococcus delphini OleTSD

Staphylococcus delphini OleTSD (SEQ ID NO: 44) is a predicted P450 fatty acid decarboxylase that converts medium chain fatty acids (e.g. linoleic acid) into the corresponding terminal olefins and that is included as an example of the current invention. A codon optimized gene (SEQ ID NO: 160) encoding for an OleTSD decarboxylase variant, including an N-terminal amino acid sequence containing a His-tag and a TEV protease cleavage site was designed and synthesized by Genscript. After gene synthesis, the protein was expressed and purified. In brief, the complete synthetic gene sequence was subcloned into a pET30a using the NdeI/XhoI cloning sites. For heterologous expression, Escherichia coli BL21 (DE3) cells were transformed with the recombinant plasmid and a single colony was inoculated into LB medium containing kanamycin (50 mg/L). Pre-starter cultures were then inoculated into a fermentor containing Magic Media (ThermoFisher, Catalog # K6803) supplemented with kanamycin (50 mg/L) and incubated at 16° C. for 72 h. At an OD₆₀₀ₙₘ=0.5-1.0, 5-aminolevulinic acid (final concentration 0.5 mM) was added. Cells were harvested by centrifugation at 5000 rpm and 4° C. and the pellets were lysed using a bacterial cell lysis buffer (B-PER—ThermoFisher, Waltham, Mass.). After centrifugation, the supernatant was collected, and the protein was purified by one-step purification using a HisPur™ Ni-NTA Spin Columns (Thermo Scientific, Catalog # 88226) and standard protocols known in the art. The protein was concentrated using a 10 kDa MW cutoff Amicon Ultra Centrifugal Filter Unit (MilliporeSigma, Catalog# UFC901024), followed by desalting using a disposable PD-10 desalting column (GE Healthcare Life Sciences, Catalog# 17085101) and a buffer containing 50 mM Tris-HCl, 500 mM NaCl, and 10% Glycerol at pH 8.0. The purified enzyme was stored at −80° C. until use.

### Example 4

### Production of Staphylococcus felis OleTSF

Staphylococcus felis OleTSF (SEQ ID NO: 60) is a predicted P450 fatty acid decarboxylase that converts medium chain fatty acids (e.g. palmitic acid) into the corresponding terminal olefins and that is included as an example of the current invention. A codon optimized gene (SEQ ID NO: 161) encoding for an OleTSF decarboxylase variant, including an N-terminal amino acid sequence containing a His-tag and a TEV protease cleavage site was designed and synthesized by Genscript. After gene synthesis, the protein was expressed and purified. In brief, the complete synthetic gene sequence was subcloned into a pET30a using the NdeI/XhoI cloning sites. For heterologous expression, Escherichia coli BL21 (DE3) cells were transformed with the recombinant plasmid and a single colony was inoculated into LB medium containing kanamycin (50 mg/L). Pre-starter cultures were then inoculated into a fermentor containing Magic Media (ThermoFisher, Catalog # K6803) supplemented with kanamycin (50 mg/L) and incubated at 16° C. for 72 h. At an OD₆₀₀ₙₘ=0.5-1.0, 5-aminolevulinic acid (final concentration 0.5 mM) was added. Cells were harvested by centrifugation at 5000 rpm and 4° C. and the pellets were lysed using a bacterial cell lysis buffer (B-PER—ThermoFisher, Waltham, Mass.). After centrifugation, the supernatant was collected, and the protein was purified by one-step purification using a HisPur™ Ni-NTA Spin Columns (Thermo Scientific, Catalog # 88226) and standard protocols known in the art. The protein was concentrated using a 10 kDa MW cutoff Amicon Ultra Centrifugal Filter Unit (MilliporeSigma, Catalog# UFC901024), followed by desalting using a disposable PD-10 desalting column (GE Healthcare Life Sciences, Catalog# 17085101) and a buffer containing 50 mM Tris-HCl, 500 mM NaCl, and 10% Glycerol at pH 8.0. The purified enzyme was stored at −80° C. until use.

### Example 5

### Production of Fictibacillus sp. S7 OleTFS

Fictibacillus sp. S7 OleTFS (SEQ ID NO: 65) is a predicted P450 fatty acid decarboxylase that converts medium chain fatty acids (e.g. palmitic acid) into the corresponding terminal olefins and that is included as an example of the current invention.v A codon optimized gene (SEQ ID NO: 162) encoding for an OleTFS decarboxylase variant, including an N-terminal amino acid sequence containing a His-tag and a TEV protease cleavage site was designed and synthesized by Genscript. After gene synthesis, the protein was expressed and purified. In brief, the complete synthetic gene sequence was subcloned into a pET30a using the NdeI/XhoI cloning sites. For heterologous expression, Escherichia coli BL21 (DE3) cells were transformed with the recombinant plasmid and a single colony was inoculated into LB medium containing kanamycin (50 mg/L). Pre-starter cultures were then inoculated into a fermentor containing Magic Media (ThermoFisher, Catalog # K6803) supplemented with kanamycin (50 mg/L) and incubated at 16 ° C. for 72 h. At an OD₆₀₀ₙₘ=0.5-1.0, 5-aminolevulinic acid (final concentration 0.5 mM) was added. Cells were harvested by centrifugation at 5000 rpm and 4° C. and the pellets were lysed using a bacterial cell lysis buffer (B-PER—ThermoFisher, Waltham, Mass.). After centrifugation, the supernatant was collected, and the protein was purified by one-step purification using a HisPur™ Ni-NTA Spin Columns (Thermo Scientific, Catalog # 88226) and standard protocols known in the art. The protein was concentrated using a 10 kDa MW cutoff Amicon Ultra Centrifugal Filter Unit (MilliporeSigma, Catalog# UFC901024), followed by desalting using a disposable PD-10 desalting column (GE Healthcare Life Sciences, Catalog# 17085101) and a buffer containing 50 mM Tris-HCl, 500 mM NaCl, and 10% Glycerol at pH 8.0. The purified enzyme was stored at −80° C. until use.

### Example 6

### Production of Staphylococcus aureus C0673 OleTSA

Staphylococcus aureus C0673 OleTSA (SEQ ID NO: 71) is a predicted P450 fatty acid decarboxylase that converts medium chain fatty acids (e.g. palmitic acid) into the corresponding terminal olefins and that is included as an example of the current invention. A codon optimized gene (SEQ ID NO: 163) encoding for an OleTSA decarboxylase variant, including an N-terminal amino acid sequence containing a His-tag and a TEV protease cleavage site was designed and synthesized by Genscript. After gene synthesis, the protein was expressed and purified. In brief, the complete synthetic gene sequence was subcloned into a pET30a using the NdeI/XhoI cloning sites. For heterologous expression, Escherichia coli BL21 (DE3) cells were transformed with the recombinant plasmid and a single colony was inoculated into LB medium containing kanamycin (50 mg/L). Pre-starter cultures were then inoculated into a fermentor containing Magic Media (ThermoFisher, Catalog # K6803) supplemented with kanamycin (50 mg/L) and incubated at 16° C. for 72 h. At an OD₆₀₀ₙₘ=0.5-1.0, 5-aminolevulinic acid (final concentration 0.5 mM) was added. Cells were harvested by centrifugation at 5000 rpm and 4° C. and the pellets were lysed using a bacterial cell lysis buffer (B-PER—ThermoFisher, Waltham, Mass.). After centrifugation, the supernatant was collected, and the protein was purified by one-step purification using a HisPur™ Ni-NTA Spin Columns (Thermo Scientific, Catalog # 88226) and standard protocols known in the art. The protein was concentrated using a 10 kDa MW cutoff Amicon Ultra Centrifugal Filter Unit (MilliporeSigma, Catalog# UFC901024), followed by desalting using a disposable PD-10 desalting column (GE Healthcare Life Sciences, Catalog# 17085101) and a buffer containing 50 mM Tris-HCl, 500 mM NaCl, and 10% Glycerol at pH 8.0. The purified enzyme was stored at −80° C. until use.

### Example 7

### Production of Auricoccus indicus OleTAI

Auricoccus indicus OleTAI (SEQ ID NO: 83) is a predicted P450 fatty acid decarboxylase that converts medium chain fatty acids (e.g. palmitic acid) into the corresponding terminal olefins and that is included as an example of the current invention. A codon optimized gene (SEQ ID NO: 164) encoding for an OleTAI decarboxylase variant, including an N-terminal amino acid sequence containing a His-tag and a TEV protease cleavage site was designed and synthesized by Genscript. After gene synthesis, the protein was expressed and purified. In brief, the complete synthetic gene sequence was subcloned into a pET30a using the NdeI/XhoI cloning sites. For heterologous expression, Escherichia coli BL21 (DE3) cells were transformed with the recombinant plasmid and a single colony was inoculated into LB medium containing kanamycin (50 mg/L). Pre-starter cultures were then inoculated into a fermentor containing Magic Media (ThermoFisher, Catalog # K6803) supplemented with kanamycin (50 mg/L) and incubated at 16° C. for 72 h. At an OD₆₀₀ₙₘ=0.5-1.0, 5-aminolevulinic acid (final concentration 0.5 mM) was added. Cells were harvested by centrifugation at 5000 rpm and 4° C. and the pellets were lysed using a bacterial cell lysis buffer (B-PER—ThermoFisher, Waltham, Mass.). After centrifugation, the supernatant was collected, and the protein was purified by one-step purification using a HisPur™ Ni-NTA Spin Columns (Thermo Scientific, Catalog # 88226) and standard protocols known in the art. The protein was concentrated using a 10 kDa MW cutoff Amicon Ultra Centrifugal Filter Unit (MilliporeSigma, Catalog# UFC901024), followed by desalting using a disposable PD-10 desalting column (GE Healthcare Life Sciences, Catalog# 17085101) and a buffer containing 50 mM Tris-HCl, 500 mM NaCl, and 10% Glycerol at pH 8.0. The purified enzyme was stored at −80° C. until use.

### Example 8

### Production of Nosocomiicoccus massiliensis OleTNM

Nosocomiicoccus massiliensis OleTNM (SEQ ID NO: 117) is a predicted P450 fatty acid decarboxylase that converts medium chain fatty acids (e.g. palmitic acid) into the corresponding terminal olefins and that is included as an example of the current invention. A codon optimized gene (SEQ ID NO: 165) encoding for an OleTNM decarboxylase variant), including an N-terminal amino acid sequence containing a His-tag and a TEV protease cleavage site was designed and synthesized by Genscript. After gene synthesis, the protein was expressed and purified. In brief, the complete synthetic gene sequence was subcloned into a pET30a using the NdeI/XhoI cloning sites. For heterologous expression, Escherichia coli BL21 (DE3) cells were transformed with the recombinant plasmid and a single colony was inoculated into LB medium containing kanamycin (50 mg/L). Pre-starter cultures were then inoculated into a fermentor containing Magic Media (ThermoFisher, Catalog # K6803) supplemented with kanamycin (50 mg/L) and incubated at 16° C. for 72 h. At an OD₆₀₀ₙₘ=0.5-1.0, 5-aminolevulinic acid (final concentration 0.5 mM) was added. Cells were harvested by centrifugation at 5000 rpm and 4° C. and the pellets were lysed using a bacterial cell lysis buffer (B-PER—ThermoFisher, Waltham, Mass.). After centrifugation, the supernatant was collected, and the protein was purified by one-step purification using a HisPur™ Ni-NTA Spin Columns (Thermo Scientific, Catalog # 88226) and standard protocols known in the art. The protein was concentrated using a 10 kDa MW cutoff Amicon Ultra Centrifugal Filter Unit (MilliporeSigma, Catalog# UFC901024), followed by desalting using a disposable PD-10 desalting column (GE Healthcare Life Sciences, Catalog# 17085101) and a buffer containing 50 mM Tris-HCl, 500 mM NaCl, and 10% Glycerol at pH 8.0. The purified enzyme was stored at −80° C. until use.

### Example 9

### Production of Pontibacillus halophilus JSM 076056 OleTPH

Pontibacillus halophilus JSM 076056 OleTPH (SEQ ID NO: 121) is a predicted P450 fatty acid decarboxylase that converts medium chain fatty acids (e.g. palmitic acid) into the corresponding terminal olefins and that is included as an example of the current invention. A codon optimized gene (SEQ ID NO: 166) encoding for an OleTPH decarboxylase variant, including an N-terminal amino acid sequence containing a His-tag and a TEV protease cleavage site was designed and synthesized by Genscript. After gene synthesis, the protein was expressed and purified. In brief, the complete synthetic gene sequence was subcloned into a pET30a using the NdeI/XhoI cloning sites. For heterologous expression, Escherichia coli BL21 (DE3) cells were transformed with the recombinant plasmid and a single colony was inoculated into LB medium containing kanamycin (50 mg/L). Pre-starter cultures were then inoculated into a fermentor containing Magic Media (ThermoFisher, Catalog # K6803) supplemented with kanamycin (50 mg/L) and incubated at 16° C. for 72 h. At an OD₆₀₀ₙₘ=0.5-1.0, 5-aminolevulinic acid (final concentration 0.5 mM) was added. Cells were harvested by centrifugation at 5000 rpm and 4° C. and the pellets were lysed using a bacterial cell lysis buffer (B-PER—ThermoFisher, Waltham, Mass.). After centrifugation, the supernatant was collected, and the protein was purified by one-step purification using a HisPur™ Ni-NTA Spin Columns (Thermo Scientific, Catalog # 88226) and standard protocols known in the art. The protein was concentrated using a 10 kDa MW cutoff Amicon Ultra Centrifugal Filter Unit (MilliporeSigma, Catalog# UFC901024), followed by desalting using a disposable PD-10 desalting column (GE Healthcare Life Sciences, Catalog# 17085101) and a buffer containing 50 mM Tris-HCl, 500 mM NaCl, and 10% Glycerol at pH 8.0. The purified enzyme was stored at −80° C. until use.

### Example 10

### Production of Macrococcus sp. DPC7161 OleTMS

Macrococcus sp. DPC7161 OleTMS (SEQ ID NO: 122) is a predicted P450 fatty acid decarboxylase that converts medium chain fatty acids (e.g. palmitic acid) into the corresponding terminal olefins and that is included as an example of the current invention. A codon optimized gene (SEQ ID NO: 167) encoding for an OleTMS decarboxylase variant, including an N-terminal amino acid sequence containing a His-tag and a TEV protease cleavage site was designed and synthesized by Genscript. After gene synthesis, the protein was expressed and purified. In brief, the complete synthetic gene sequence was subcloned into a pET30a using the NdeI/XhoI cloning sites. For heterologous expression, Escherichia coli BL21 (DE3) cells were transformed with the recombinant plasmid and a single colony was inoculated into LB medium containing kanamycin (50 mg/L). Pre-starter cultures were then inoculated into a fermentor containing Magic Media (ThermoFisher, Catalog # K6803) supplemented with kanamycin (50 mg/L) and incubated at 16° C. for 72 h. At an OD₆₀₀ₙₘ=0.5-1.0, 5-aminolevulinic acid (final concentration 0.5 mM) was added. Cells were harvested by centrifugation at 5000 rpm and 4° C. and the pellets were lysed using a bacterial cell lysis buffer (B-PER—ThermoFisher, Waltham, Mass.). After centrifugation, the supernatant was collected, and the protein was purified by one-step purification using a HisPur™ Ni-NTA Spin Columns (Thermo Scientific, Catalog # 88226) and standard protocols known in the art. The protein was concentrated using a 10 kDa MW cutoff Amicon Ultra Centrifugal Filter Unit (MilliporeSigma, Catalog# UFC901024), followed by desalting using a disposable PD-10 desalting column (GE Healthcare Life Sciences, Catalog# 17085101) and a buffer containing 50 mM Tris-HCl, 500 mM NaCl, and 10% Glycerol at pH 8.0. The purified enzyme was stored at −80° C. until use.

### Example 11

### Production of Staphylococcus massiliensis S46 OleTSM

Staphylococcus massiliensis S46 OleTSM (SEQ ID NO: 156) is a P450 fatty acid decarboxylase that converts medium chain fatty acids (e.g. palmitic acid) into the corresponding terminal olefins and that is included as an example of the current invention. A codon optimized gene (SEQ ID NO: 168) encoding for an OleTSM decarboxylase variant, without the initial N-terminal 29 amino acids including an N-terminal amino acid sequence containing a His-tag and a TEV protease cleavage site was designed and synthesized by Genscript.

In brief, the complete synthetic gene sequence was subcloned into a pET30a vector using the NdeI and HindIII cloning sites for heterologous expression. Escherichia coli C41 (DE3) cells were co-transformed with the recombinant plasmid and with plasmid pTf16. A single colony was inoculated into TB medium containing kanamycin and chloramphenicol. Cultures were incubated at 15° C. for 16 h at 200 rpm and L-arabinose (final concentration 0.1%), δ-aminolevulinic acid (final concentration 0.25 mM) and isopropyl β-D-1-thiogalactopyranoside (IPTG, final concentration 1 mM) were added to induce protein expression. Cells were harvested by centrifugation at 5000 rpm and 4° C. and the pellets were lysed by sonication. After centrifugation, the supernatant was collected and the protein was purified by one-step purification using a nickel affinity column and standard protocols known in the art. The protein was stored in a buffer containing 50 mM Tris-HCl, 500 mM NaCl, and 10% Glycerol at pH 8.0. The final protein concentration was 1.65 mg/ mL as determined by Bradford protein assay with BSA as a standard (ThermoFisher, catalog # 23236).

### Comparative Example A

### Production of Jeotgalicoccus sp. OleTJE

Jeotgalicoccus sp. OleTJE (SEQ ID NO: 1) is a P450 fatty acid decarboxylase that converts medium chain fatty acids (e.g. palmitic acid) into the corresponding terminal olefins and is included as a comparative example of the present invention. A codon optimized gene (SEQ ID NO: 152) encoding for a Jeotgalicoccus sp. OleTJE variant, including an N-terminal amino acid sequence containing a His-tag and a TEV protease cleavage site (SEQ ID NO: 153), was designed and synthesized. After gene synthesis, the protein was expressed and purified. In brief, the complete synthetic gene sequence was subcloned into a pET30a vector by GenScript (Piscataway, N.J.). For heterologous expression. Escherichia coli BL21 Star™ (DE3) pLysS cells were transformed with the recombinant plasmid and a single colony was inoculated into LB medium containing kanamycin and chloramphenicol. Pre-starter cultures were then inoculated into a fermentor (BioFlo/CelliGen 310; NewBrunswick, Hamburg, Germany) containing LB medium supplemented with kanamycin and chloramphenicol and incubated at 25° C. At an OD₆₀₀ₙₘ=0.4, isopropyl β-D-1-thiogalactopyranoside (IPTG) (final concentration 0.1 mM) and 5-aminolevulinic acid (final concentration 0.5 mM) were added to induce protein expression. Cells were harvested by centrifugation at 5000 rpm and 4° C. and the pellets were lysed by a bacterial cell lysis buffer (B-PER—ThermoFisher, Waltham, Mass.). After centrifugation, the supernatant was collected, and the protein was purified by one-step purification using Ni-NTA agarose resin (Qiagen, Hilden, Germany; catalog # 30230) and standard protocols known in the art. The protein was dialyzed using a membrane with 10 kDa MW cutoff against a buffer containing 50 mM Tris-HCl and 10% Glycerol at pH 8.0. The final protein concentration was 1.1 mg/mL as determined by Modified Lowry protein assay with BSA as a standard (ThermoFisher Scientific, Waltham, Mass.).

### Comparative Example B

### Production of Macrococcus goetzii OleTMG

Macrococcus goetzii OleTMG (SEQ ID NO: 20) is a predicted P450 fatty acid decarboxylase that converts fatty acids into the corresponding terminal olefins and is included as a comparative example of the present invention. A codon optimized gene (SEQ ID NO: 169) encoding for an OleTMG decarboxylase variant, including an N-terminal amino acid sequence containing a His-tag and a TEV protease cleavage site was designed and synthesized by Genscript. After gene synthesis, the protein was expressed and purified. In brief, the complete synthetic gene sequence was subcloned into a pET30a using the NdeI/XhoI cloning sites. For heterologous expression, Escherichia coli BL21 (DE3) cells were transformed with the recombinant plasmid and a single colony was inoculated into LB medium containing kanamycin (50 mg/L). Pre-starter cultures were then inoculated into a fermentor containing Magic Media (ThermoFisher, Catalog # K6803) supplemented with kanamycin (50 mg/L) and incubated at 16° C. for 72 h. At an OD₆₀₀ₙₘ=0.5-1.0, 5-aminolevulinic acid (final concentration 0.5 mM) was added. Cells were harvested by centrifugation at 5000 rpm and 4° C. and the pellets were lysed using a bacterial cell lysis buffer (B-PER—ThermoFisher, Waltham, Mass.). After centrifugation, the supernatant was collected, and the protein was purified by one-step purification using a HisPur™ Ni-NTA Spin Columns (Thermo Scientific, Catalog # 88226) and standard protocols known in the art. The protein was concentrated using a 10 kDa MW cutoff Amicon Ultra Centrifugal Filter Unit (MilliporeSigm, Catalog# UFC901024), followed by desalting using a disposable PD-10 desalting column (GE Healthcare Life Sciences, Catalog# 17085101) and a buffer containing 50 mM Tris-HCl, 500 mM NaCl, and 10% Glycerol at pH 8.0. The purified enzyme was stored at −80° C. until use.

### Comparative Example C

### Production of Macrococcus lamae OleTMA

Macrococcus lamae OleTMA (SEQ ID NO: 21) is a predicted P450 fatty acid decarboxylase that converts fatty acids into the corresponding terminal olefins and is included as a comparative example of the present invention. A codon optimized gene (SEQ ID NO: 170) encoding for an OleTMA decarboxylase variant, including an N-terminal amino acid sequence containing a His-tag and a TEV protease cleavage site was designed and synthesized by Genscript. After gene synthesis, the protein was expressed and purified. In brief, the complete synthetic gene sequence was subcloned into a pET30a using the NdeI/XhoI cloning sites. For heterologous expression, Escherichia coli BL21 (DE3) cells were transformed with the recombinant plasmid and a single colony was inoculated into LB medium containing kanamycin (50 mg/L). Pre-starter cultures were then inoculated into a fermentor containing Magic Media (ThermoFisher, Catalog # K6803) supplemented with kanamycin (50 mg/L) and incubated at 16° C. for 72 h. At an OD₆₀₀ₙₘ=0.5-1.0, 5-aminolevulinic acid (final concentration 0.5 mM) was added. Cells were harvested by centrifugation at 5000 rpm and 4° C. and the pellets were lysed using a bacterial cell lysis buffer (B-PER—ThermoFisher, Waltham, Mass.). After centrifugation, the supernatant was collected, and the protein was purified by one-step purification using a HisPur™ Ni-NTA Spin Columns (Thermo Scientific, Catalog # 88226) and standard protocols known in the art. The protein was concentrated using a 10 kDa MW cutoff Amicon Ultra Centrifugal Filter Unit (MilliporeSigm, Catalog# UFC901024), followed by desalting using a disposable PD-10 desalting column (GE Healthcare Life Sciences, Catalog# 17085101) and a buffer containing 50 mM Tris-HCl, 500 mM NaCl, and 10% Glycerol at pH 8.0. The purified enzyme was stored at −80° C. until use.

### Comparative Example D

### Production of Salinicoccus sp. CT19 OleTSS

Salinicoccus sp. CT19 OleTSS (SEQ ID NO: 42) is a predicted P450 fatty acid decarboxylase that converts fatty acids into the corresponding terminal olefins and is included as a comparative example of the present invention. A codon optimized gene (SEQ ID NO: 171) encoding for an OleTSS decarboxylase variant, including an N-terminal amino acid sequence containing a His-tag and a TEV protease cleavage site was designed and synthesized by Genscript. After gene synthesis, the protein was expressed and purified. In brief, the complete synthetic gene sequence was subcloned into a pET30a using the NdeI/XhoI cloning sites. For heterologous expression, Escherichia coli BL21 (DE3) cells were transformed with the recombinant plasmid and a single colony was inoculated into LB medium containing kanamycin (50 mg/L). Pre-starter cultures were then inoculated into a fermentor containing Magic Media (ThermoFisher, Catalog # K6803) supplemented with kanamycin (50 mg/L) and incubated at 16° C. for 72 h. At an OD₆₀₀ₙₘ=0.5-1.0, 5-aminolevulinic acid (final concentration 0.5 mM) was added. Cells were harvested by centrifugation at 5000 rpm and 4° C. and the pellets were lysed using a bacterial cell lysis buffer (B-PER—ThermoFisher, Waltham, Mass.). After centrifugation, the supernatant was collected, and the protein was purified by one-step purification using a HisPur™ Ni-NTA Spin Columns (Thermo Scientific, Catalog # 88226) and standard protocols known in the art. The protein was concentrated using a 10 kDa MW cutoff Amicon Ultra Centrifugal Filter Unit (MilliporeSigm, Catalog# UFC901024), followed by desalting using a disposable PD-10 desalting column (GE Healthcare Life Sciences, Catalog# 17085101) and a buffer containing 50 mM Tris-HCl, 500 mM NaCl, and 10% Glycerol at pH 8.0. The purified enzyme was stored at −80° C. until use.

### Comparative Example E

### Production of Aliicoccus persicus OleTAP

Aliicoccus persicus OleTAP (SEQ ID NO: 84) is a predicted P450 fatty acid decarboxylase that converts fatty acids into the corresponding terminal olefins and is included as a comparative example of the present invention. A codon optimized gene (SEQ ID NO: 172) encoding for an OleTAP decarboxylase variant, including an N-terminal amino acid sequence containing a His-tag and a TEV protease cleavage site was designed and synthesized by Genscript. After gene synthesis, the protein was expressed and purified. In brief, the complete synthetic gene sequence was subcloned into a pET30a using the NdeI/XhoI cloning sites. For heterologous expression, Escherichia coli BL21 (DE3) cells were transformed with the recombinant plasmid and a single colony was inoculated into LB medium containing kanamycin (50 mg/L). Pre-starter cultures were then inoculated into a fermentor containing Magic Media (ThermoFisher, Catalog # K6803) supplemented with kanamycin (50 mg/L) and incubated at 16° C. for 72 h. At an OD₆₀₀ₙₘ=0.5-1.0, 5-aminolevulinic acid (final concentration 0.5 mM) was added. Cells were harvested by centrifugation at 5000 rpm and 4° C. and the pellets were lysed using a bacterial cell lysis buffer (B-PER—ThermoFisher, Waltham, Mass.). After centrifugation, the supernatant was collected, and the protein was purified by one-step purification using a HisPur™ Ni-NTA Spin Columns (Thermo Scientific, Catalog # 88226) and standard protocols known in the art. The protein was concentrated using a 10 kDa MW cutoff Amicon Ultra Centrifugal Filter Unit (MilliporeSigm, Catalog# UFC901024), followed by desalting using a disposable PD-10 desalting column (GE Healthcare Life Sciences, Catalog# 17085101) and a buffer containing 50 mM Tris-HCl, 500 mM NaCl, and 10% Glycerol at pH 8.0. The purified enzyme was stored at −80° C. until use.

### Comparative Example F

### Production of Salinicoccus qingdaonensis OleTSQ

Salinicoccus qingdaonensis OleTSQ (SEQ ID NO: 100) is a predicted P450 fatty acid decarboxylase that converts fatty acids into the corresponding terminal olefins and is included as a comparative example of the present invention. A codon optimized gene (SEQ ID NO: 173) encoding for an OleTSQ decarboxylase variant, including an N-terminal amino acid sequence containing a His-tag and a TEV protease cleavage site was designed and synthesized by Genscript.

After gene synthesis, the protein was expressed and purified. In brief, the complete synthetic gene sequence was subcloned into a pET30a using the NdeI/XhoI cloning sites. For heterologous expression, Escherichia coli BL21 (DE3) cells were transformed with the recombinant plasmid and a single colony was inoculated into LB medium containing kanamycin (50 mg/L). Pre-starter cultures were then inoculated into a fermentor containing Magic Media (ThermoFisher, Catalog # K6803) supplemented with kanamycin (50 mg/L) and incubated at 16° C. for 72 h. At an OD₆₀₀ₙₘ=0.5-1.0, 5-aminolevulinic acid (final concentration 0.5 mM) was added. Cells were harvested by centrifugation at 5000 rpm and 4° C. and the pellets were lysed using a bacterial cell lysis buffer (B-PER—ThermoFisher, Waltham, Mass.). After centrifugation, the supernatant was collected, and the protein was purified by one-step purification using a HisPur™ Ni-NTA Spin Columns (Thermo Scientific, Catalog # 88226) and standard protocols known in the art. The protein was concentrated using a 10 kDa MW cutoff Amicon Ultra Centrifugal Filter Unit (MilliporeSigm, Catalog# UFC901024), followed by desalting using a disposable PD-10 desalting column (GE Healthcare Life Sciences, Catalog# 17085101) and a buffer containing 50 mM Tris-HCl, 500 mM NaCl, and 10% Glycerol at pH 8.0. The purified enzyme was stored at −80° C. until use.

### Example 12

### Enzyme Activity Assay

Reactions of oleic acid and/or linoleic acid with the earlier described OleT enzymes produced as described in examples 1 to 11 and comparative example A, were performed as follows. Aliquots of sodium oleate or sodium linoleate (final concentration 100 μM) and enzyme (final concentration 6 ppm) were resuspended in buffer (50 mM phosphate, 500 mM NaCl, and 10% glycerol at pH 7.4). The reaction was started by addition of hydrogen peroxide (final concentration 220 μM) and the solutions were incubated at 30° C. Aliquots of 100 μL of the reaction solutions were collected at different time points and mixed with 900 μL of isopropyl alcohol to stop the reactions. Analysis of the samples was performed by reversed-phase LC/MS/MS to determine the concentrations of oleate remaining in the solutions. The TON numbers (in s⁻¹) were calculated as the ratio between the initial rate of substrate conversion (in μM/s) and the concentration of enzyme (in μM). Finally, the improvement factors were calculated as the ratio of the TON number for the specific enzyme and the TON number for Jeotgalicoccus sp. OleTJE (SEQ ID NO: 1). The results are summarized in Table 2.

TABLE 2
Conversion of sodium oleate by OleT decarboxylases
at different time points.

| SEQ ID | | Linoleic- | Oleic- |
|---|---|---|---|
| NO: | Organism | Improv. Factor | Improv. Factor |
| 1* | Jeotgalicoccus sp. | 1.00 | 1.00 |
| 2 | Micrococcus lylae | 86.50 | 76.67 |
| 22 | Macrococcus bovicus | 0.00 | 2.00 |
| 44 | Staphylococcus delphini | 2.55 | 0.86 |
| 60 | Staphylococcus felis | 24.00 | 0.10 |
| 65 | Fictibacillus sp. S7 | 20.50 | 5.11 |
| 71 | Staphylococcus aureus C0673 | 10.35 | 3.22 |
| 83 | Auricoccus indicus | 12.00 | 0.33 |
| 117 | Nosocomiicoccus massiliensis | 5.00 | 0.77 |
| 121 | Pontibacillus halophilus JSM | 0.00 | 2.33 |
| | 076056 | | |
| 122 | Macrococcus sp. DPC7161 | 42.00 | 18.44 |
| 156 | Staphylococcus massiliensis S46 | 3.75 | 2.22 |
| 20* | Macrococcus goetzii | 0.00 | 1.44 |
| 21* | Macrococcus lamae | 0.00 | 1.39 |
| 42* | Salinicoccus sp. CT19 | 0.00 | 1.33 |
| 84* | Aliicoccus persicus | 0.00 | 1.11 |
| 100* | Salinicoccus qingdaonensis | 0.00 | 0.43 |

*Comparative

### Example 13

### Sequence Similarity Network

Sequence similarity networks (SSN) are multidimensional versions of the more traditional one-dimensional BLAST analysis. In SSN, pairwise sequence relationships among different proteins are visualized. Each protein is illustrated as a “node”, while each node is connected to other nodes by “edges”. Only nodes representing proteins that are similar enough, based on amino acid sequence identity, are connected by edges. Thus, groups of highly similar proteins form clusters in an SSN diagram.

An SSN for OleT decarboxylases was created using the EFI web tools (https://efi.igb.illinois.edu/. Rémi Zallot, Nils Oberg, and John A. Gerlt, The EFI Web Resource for Genomic Enzymology Tools: Leveraging Protein, Genome, and Metagenome Databases to Discover Novel Enzymes and Metabolic Pathways. Biochemistry 2019 58 (41), 4169-4182. https://doi.org/10.1021/acs.biochem.9b00735). Proteins with more than about 85% sequence identity were grouped together in clusters (see FIGURE and Table 3). Representative sequences of every cluster were selected for kinetic characterization (see Table 2). Enzymes that convert the fatty acids at a higher rate (i.e. higher TON) compared to the rate of Jeotgalicoccus sp. OleT (SEQ ID NO: 1) are preferred in the current application. For instance, several decarboxylases (SEQ ID NO: 2, 122, 60, 65, 83, 71, 117, 156, 44) convert linoleic acid at least twice faster than Jeotgalicoccus sp. OleT (SEQ ID NO: 1) and are preferred decarboxylases of the current application. On a separate example, several decarboxylases (SEQ ID NO: 2, 122, 65, 71, 121, 156, 22) convert oleic acid at least twice faster than Jeotgalicoccus sp. OleT (SEQ ID NO: 1) and are also preferred decarboxylases of the current application.

SSNs have been used to identify and describe isofunctional families within enzyme families, e.g. clusters with different substrate specificity, providing an overview of sequence-function space (John A. Gerlt, Genomic enzymology: Web tools for leveraging protein family sequence-function space and genome context to discover novel functions, Biochemistry. Volume 56, 2017, Pages 4293-4308, https://doi.org/10.1021/acs.biochem.7b00614). It stands within reason that enzyme candidates within the same cluster can have similar properties due to the high level of sequence similarity. Thus, in addition to the preferred enzymes, other decarboxylases within their corresponding clusters are included as part of the current invention (see Table 3). For instance, decarboxylases with SEQ ID NO 3, 5, 4, 7, 6, 8, 9, 10, 11, 12, 13, 14, 15, and 151 are very similar to SEQ ID NO 2, as their nodes are connected by edges in cluster 5 of the SSN (see FIGURE and Table 3), and are therefore included as part of the current invention. Same arguments are valid for enzymes included in clusters 1, 3, 9, 17, 27, 40, 45, 52, 3*, and 109*.

TABLE 3
List of clusters of OleT decarboxylases based on SSN analysis.

| | SEQ | |
|---|---|---|
| Cluster | ID NO | Other SEQ ID NO in Cluster |
| 1 | 44 | 39, 40, 41, 43, 45, 46, 47, 48, 49, 50, 85, 126, 127, |
| | | 128, 130, 131, 132, 133, 134, 135, 136, 137, 138, |
| | | 139, 140, 141, 142, 143, 144, 145, 146 |
| 2 | 104 | 86, 87, 88, 89, 90, 91, 94, 95, 96, 97, 98, 99, 102, |
| | | 103, 105, 106, 107, 108, 109, 110, 111, 112, 113 |
| 3 | 71 | 67, 68, 69, 70, 72, 73, 74, 75, 76, 77, |
| | | 78, 79, 80, 81, 123, 124, 125 |
| 4 | 1 | 148, 149, 150, 154 |
| 4 | 158 | 1, 148, 149, 150 |
| 5 | 2 | 3, 5, 4, 7, 6, 8, 9, 10, 11, 12, 13, 14, 15, 151 |
| 9 | 60 | 54, 55, 56, 57, 58, 59, 61, 62, 63 |
| 10 | 42 | 25, 26, 27, 28, 51, 52, 92, 93 |
| 17 | 117 | 114, 115, 116, 118, 119 |
| 19 | 20 | 16, 17, 18, 19 |
| 27 | 22 | 23, 24, 129 |
| 28 | 100 | 53, 101 |
| 40 | 65 | 64 |
| 45 | 83 | 82 |
| 52 | 156 | 157 |
| 3* | 121 | |
| 108* | 21 | |
| 109* | 122 | |
| 110* | 84 | |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A consumer product composition comprising a P450 fatty acid decarboxylase; wherein said decarboxylase comprises a polypeptide sequence having at least about 80% identity to one or more sequences selected from the group consisting of: SEQ ID NO: 2, 22, 44, 60, 65, 71, 83, 117, 121, 122, 156, and their functional fragments thereof; preferably SEQ ID NO: 2, 60, 65, 71, 83, 122, and their functional fragments.

2. The consumer product composition according to claim 1, wherein said decarboxylase comprises a polypeptide sequence having at least about 80% identity to one or more sequences selected from the group consisting of: SEQ ID NO: 2, 122, and their functional fragments.

3. The consumer product composition according to claim 2, wherein said decarboxylase comprises a polypeptide sequence having at least about 80% identity to SEQ ID NO: 2 and its functional fragments.

4. The consumer product composition according to claim 1, wherein said decarboxylase comprises a polypeptide sequence having at least about 90%, 95%, 98%, 100% identity to one or more sequences selected from the group consisting of: SEQ ID NO: 2, 3, 4, 5, 6, 7, 8, 9, 10, 11, 12, 13, 14, 15, 22, 23, 24, 39, 40, 41, 43, 44, 44, 45, 46, 47, 48, 49, 50, 54, 55, 56, 57, 58, 59, 60, 60, 61, 62, 63, 64, 65, 67, 68, 69, 70, 71, 71, 72, 73, 74, 75, 76, 77, 78, 79, 80, 81, 82, 83, 85, 114, 115, 116, 117, 117, 118, 119, 121, 122, 123, 124, 125, 126, 127, 128, 129, 130, 131, 132, 133, 134, 135, 136, 137, 138, 139, 140, 141, 142, 143, 144, 145, 146, 151, 156, 157, and their functional fragments thereof.

5. The consumer product composition according to claim 4, wherein said decarboxylase comprises a polypeptide sequence having at least about 90%, 95%, 98%, 100% identity to one or more sequences selected from the group consisting of SEQ ID NO: 2, 3, 5, 4, 7, 6, 8, 9, 10, 11, 12, 13, 14, 15, 122, 151, and their functional fragments thereof.

6. The consumer product composition according to claim 1, further comprising one or more co-enzymes selected from the group consisting of: fatty-acid peroxidases (EC 1.11.1.3), unspecific peroxygenases (EC 1.11.2.1), plant seed peroxygenases (EC 1.11.2.3), fatty acid peroxygenases (EC1.11.2.4), linoleate diol synthases (EC 1.13.11.44), 5,8-linoleate diol synthases (EC 1.13.11.60 and EC 5.4.4.5), 7,8-linoleate diol synthases (EC 1.13.11.60 and EC 5.4.4.6), 9,14-linoleate diol synthases (EC 1.13.11.B1), 8,11-linoleate diol synthases, oleate diol synthases, other linoleate diol synthases, unspecific monooxygenase (EC 1.14.14.1), alkane 1-monooxygenase (EC 1.14.15.3), oleate 12-hydroxylases (EC 1.14.18.4), fatty acid amide hydrolases (EC 3.5.1.99), fatty acid photodecarboxylases (EC 4.1.1.106), oleate hydratases (EC 4.2.1.53), linoleate isomerases (EC 5.2.1.5), linoleate (10E,12Z)-isomerases (EC 5.3.3.B2), non-heme fatty acid decarboxylases (UndA-like), alpha-dioxygenases, amylases, lipases, proteases, cellulases, and mixtures thereof; preferably fatty-acid peroxidases (EC 1.11.1.3), unspecific peroxygenases (EC 1.11.2.1), plant seed peroxygenases (EC 1.11.2.3), and fatty acid peroxygenases (EC1.11.2.4), non-heme fatty acid decarboxylases (UndA-like), alpha-dioxygenases, and mixtures thereof.

7. The consumer product composition according to claim 1, wherein said one or more P450 fatty acid decarboxylases are present in an amount of from about 0.0001 wt % to about 1 wt %, by weight of the consumer product composition, based on active protein.

8. The consumer product composition according to claim 7, wherein said one or more P450 fatty acid decarboxylases are present in an amount of from about preferably from about 0.001 wt % to about 0.2 wt %, by weight of the consumer product composition, based on active protein.

9. The consumer product composition according to claim 1, further comprising a surfactant.

10. The consumer product composition according to claim 9, wherein the surfactant is present in an amount of from about 1 wt % to about 60 wt %, by weight of the consumer product composition.

11. The consumer product composition according to claim 10, wherein the surfactant is present in an amount of from about 5 wt % to about 50 wt %, by weight of the consumer product composition.

12. The consumer product composition according to claim 9, wherein said surfactant comprises one or more anionic surfactants and one or more co-surfactants selected from the group consisting of amphoteric surfactant, zwitterionic surfactant, and mixtures thereof.
